(19) United States
(12) Patent Application Publication
(10) Pub. No.: US 2011/0256135 A1
(43) Pub. Date: Oct. 20, 2011

(54) ANTI-NERVE GROWTH FACTOR (NGF) ANTIBODY COMPOSITIONS

(76) Inventors: Wolfgang FRAUNHOFER, Gurnee, IL (US); Ravi Chari, Worcester, MA (US); Vineet Kumar, Storrs, CT (US); Rainer Saedler, Mannheim (DE); Michael Siedler, Boston, MA (US); William B. Stine, Shrewsbury, MA (US); Carsten Weber, Maxdorf (DE)

(21) Appl. No.: 13/049,473

(22) Filed: Mar. 16, 2011

Related U.S. Application Data
(60) Provisional application No. 61/314,984, filed on Mar. 17, 2010.

(51) Int. Cl. A61K 39/395; A61M 15/00; A61M 5/30; A61P 25/04; A61M 5/178

(52) U.S. Cl. 424/135.1; 424/158.1; 424/133.1; 424/145.1; 424/136.1; 604/187; 604/68; 128/203.12

(57) ABSTRACT

The present invention relates to stable compositions of anti-NGF antibodies, and antigen-binding fragments thereof, and their uses in the prevention and/or treatment of various diseases and disorders in which NGF activity is detrimental, e.g., pain disorders.

This application claims priority to U.S. provisional application No. 61/314,984, filed on Mar. 17, 2010, the contents of which are incorporated herein by reference.

### BACKGROUND OF THE INVENTION

Nerve growth factor (NGF) is a secreted protein that was discovered over 50 years ago as a molecule that promotes the survival and differentiation of sensory and sympathetic neurons. The beta chain of NGF is solely responsible for the nerve growth stimulating activity of NGF. The beta chain homodimerizes and is incorporated into a larger protein complex. NGF is a member of a family of neurotrophic factors known as neurotrophins. NGF binds with high affinity to a tropomyosin receptor kinase known as TrkA. NGF is also capable of binding a receptor known as p75^{NTR}, a member of the tumor necrosis factor receptor superfamily, which also interacts with other neurotrophins. The structure and function of NGF is reviewed in, for example, Sofroniew, M. V. et al. (2001) Annu. Rev. Neurosci. 24:1217-1281; Weismann, C. and de Vos, A. M. (2001) Cell. Mol. Life. Sci. 58:748-759; Fahnestock, M. (1991) Curr. Top. Microbiol. Immunol. 165:1-26.

Although NGF was originally identified for its ability to promote the survival and differentiation of neurons, there is growing evidence that these developmental effects are only one aspect of the biology of NGF. In particular, NGF has been implicated in the transmission and maintenance of persistent or chronic pain. For example, both local and systemic administration of NGF have been shown to elicit hyperalgesia and allodynia (Lewin, G. R. et al. (1994) Eur. J. Neurosci. 6:1903-1912). Intravenous infusion of NGF in humans produces a whole body myalgia while local administration evokes injection site hyperalgesia and allodynia in addition to the systemic effects (Apfel, S. C. et al. (1998) Neurology 51:695-702). Furthermore, in certain forms of cancer, excess NGF facilitates the growth and infiltration of nerve fibers with induction of cancer pain (Zhu, Z. et al. (1999) J. Clin. Oncol. 17:241-228).

The involvement of NGF in chronic pain has led to considerable interest in therapeutic approaches based on inhibiting the effects of NGF (see e.g., Saragovi, H. U. and Gehring, K. (2000) Trends Pharmacol. Sci. 21:93-98). For example, a soluble form of the TrkA receptor was used to block the activity of NGF, which was shown to significantly reduce the formation of neuromas, responsible for neuropathic pain, without damaging the cell bodies of the lesioned neurons (Kryger, G. S. et al. (2001) J. Hand Surg. (Am.) 26:635-644).

Another approach to neutralizing NGF activity is the use of anti-NGF antibodies, examples of which antibodies have been described (see e.g., PCT Publication Nos. WO 2001/78698, WO 2001/64247, WO 2002/096458, WO 2004/032870, WO 2005/061540, WO 2006/131951, WO 2006/110883, U.S. Pat. No. 7,449,616; U.S. Publication Nos. US 20050074821, US 20080033157, US 20080182978 and US 20090041717). In animal models of neuropathic pain (e.g., nerve trunk or spinal nerve ligation) systemic injection of neutralizing antibodies to NGF prevents both allodynia and hyperalgesia (Ramer, M. S, and Bisby, M. A. (1999) Eur. J. Neurosci. 11:837-846; Ro, L. S. et al. (1999) Pain 79:265-274). Furthermore, treatment with a neutralizing anti-NGF antibody produces significant pain reduction in a murine cancer pain model (Sevcik, M. A. et al. (2005) Pain 115:128-141).

Earlier formulations containing anti-NGF antibodies (e.g., PG110) have suffered from physical instability of the antibody in the formulation, as reflected by severe visible particle formation and precipitation phenomena. Thus, there is a need in the art for formulations containing anti-NGF antibodies which maintain physical stability and which reduce particle formation susceptibility.

### SUMMARY OF THE INVENTION

The present invention, is based, at least in part, on the discovery of novel formulations containing anti-NGF antibodies (e.g., the humanized PG110 antibody) which formulations are physically stable and do not suffer from particle formation susceptibilities.

Accordingly, the present invention provides pharmaceutical compositions comprising: (a) an anti-nerve growth factor (NGF) antibody, or antigen binding fragment thereof, (b) a histidine buffer at a concentration of about 5 to about 60 mM; and (c) polysorbate 80 at a concentration of about 0.01% to about 0.1%; wherein the pH of the composition is about 5.0 to about 6.0. In certain embodiments, the composition further comprises a sugar and/or polyol, such as those described herein. In other embodiments, the composition does not comprise a polyol or sugar. In yet other embodiments, the composition does not comprise methionine.

In certain embodiments, the present invention provides a pharmaceutical compositions consisting of, or consisting essentially of, (a) an anti-nerve growth factor (NGF) antibody, or antigen binding fragment thereof, (b) a histidine buffer at a concentration of about 5 to about 60 mM; and (c) polysorbate 80 at a concentration of about 0.01% to about 0.1%; wherein the pH of the composition is about 5.0 to about 6.0.

In certain embodiments, the present invention provides a pharmaceutical compositions consisting of, or consisting essentially of, (a) an anti-nerve growth factor (NGF) antibody, or antigen binding fragment thereof, (b) a histidine buffer at a concentration of about 5 to about 60 mM; (c) polysorbate 80 at a concentration of about 0.01% to about 0.1%; and (d) a polylol and/or a sugar; wherein the pH of the composition is about 5.0 to about 6.0.

In certain embodiments, the present invention provides a pharmaceutical compositions consisting of, or consisting essentially of, (a) an anti-nerve growth factor (NGF) antibody, or antigen binding fragment thereof, (b) a histidine buffer at a concentration of about 5 to about 60 mM; (c) polysorbate 80 at a concentration of about 0.01% to about 0.1%; and (d) a polyol; wherein the pH of the composition is about 5.0 to about 6.0.

In certain embodiments, the present invention provides a pharmaceutical compositions consisting of, or consisting essentially of, (a) an anti-nerve growth factor (NGF) antibody, or antigen binding fragment thereof, (b) a histidine buffer at a concentration of about 5 to about 60 mM; (c) polysorbate 80 at a concentration of about 0.01% to about 0.1%; and (d) a sugar; wherein the pH of the composition is about 5.0 to about 6.0.

In certain embodiments, the pharmaceutical composition of the invention is a liquid pharmaceutical composition. In other embodiments, the pharmaceutical composition is suitable for lyophilization. Accordingly, the invention further provides lyophilized pharmaceutical compositions comprising (a) about 1 to about 240 mg of an anti-NGF antibody, or antigen binding fragment thereof; (b) about 1 to about 10 mg of histidine; and (c) about 0.1 to about 0.4 mg of polysorbate 80. In certain embodiments, the lyophilized composition further comprises a sugar and/or polyol. In other embodiments, the lyophilized composition does not comprise a polyol or sugar.

In certain embodiments, the present invention provides a pharmaceutical compositions consisting of, or consisting essentially of, (a) about 1 to about 240 mg of an anti-NGF antibody, or antigen binding fragment thereof; (b) about 1 to about 10 mg of histidine; and (c) about 0.1 to about 0.4 mg of polysorbate 80.

In other embodiments, the present invention provides a pharmaceutical compositions consisting of, or consisting essentially of, (a) about 1 to about 240 mg of an anti-NGF antibody, or antigen binding fragment thereof; (b) about 1 to about 10 mg of histidine; (c) about 0.1 to about 0.4 mg of polysorbate 80; and (d) about 1 to about 100 mg of a polylol and/or about 1 to about 100 mg of a sugar.

In other embodiments, the present invention provides a pharmaceutical compositions consisting of, or consisting essentially of, (a) about 1 to about 240 mg of an anti-NGF antibody, or antigen binding fragment thereof; (b) about 1 to about 10 mg of histidine; (c) about 0.1 to about 0.4 mg of polysorbate 80; and (d) about 1 to about 100 mg of a polylol.

In still other embodiments, the present invention provides a pharmaceutical compositions consisting of, or consisting essentially of, (a) about 1 to about 240 mg of an anti-NGF antibody, or antigen binding fragment thereof; (b) about 1 to about 10 mg of histidine; (c) about 0.1 to about 0.4 mg of polysorbate 80; and (d) about 1 to about 100 mg of a sugar. In certain embodiments, the anti-NGF antibody, or antigen-binding portion thereof, binds to human NGF. In other embodiments, the antibody, or antigen-binding portion thereof, comprises a human IgG4 constant region, wherein the human IgG4 constant region comprises a hinge region mutation. Preferably, the hinge region mutation in the IgG4 constant region comprises mutation of serine at amino acid position 108 of SEQ ID NO: 9 (which shows the wild type amino acid sequence of the human IgG4 constant region). More preferably, the serine at amino acid position 108 of SEQ ID NO: 9 is mutated to proline. In a preferred embodiment, the human IgG4 constant region of the anti-NGF antibody comprises the amino acid sequence of SEQ ID NO: 10.

A preferred anti-NGF antibody contained in the compositions of the invention is antibody PG110, the heavy chain amino acid sequence of which is shown in SEQ ID NO: 13 and the light chain amino acid sequence of which is shown in SEQ ID NO: 16. In another embodiment, the invention provides compositions containing an anti-NGF antibody comprising a heavy chain encoded by the nucleotide sequence of SEQ ID NO: 11 and a light chain encoded by the nucleotide sequence of SEQ ID NO: 14. In another embodiment, the anti-NGF antibody comprises a heavy chain variable region comprising the amino acid sequence of SEQ ID NO: 1 (which shows the heavy chain variable region of PG110). In another embodiment, the anti-NGF antibody comprises a light chain variable region comprising the amino acid sequence of SEQ ID NO: 2 (which shows the light chain variable region of PG110). In yet another embodiment, the anti-NGF antibody comprises a heavy chain variable region comprising the amino acid sequence of SEQ ID NO: 1 and a light chain variable region comprising the amino acid sequence of SEQ ID NO: 2. In still another embodiment, the anti-NGF antibody competes for binding to NGF with an antibody comprising a heavy chain variable region comprising the amino acid sequence of SEQ ID NO: 1 and a light chain variable region comprising the amino acid sequence of SEQ ID NO: 2.

In another embodiment, the anti-NGF antibody comprises a heavy chain variable region comprising CDRs 1, 2 and 3 having the amino acid sequences of SEQ ID NOs: 3, 4 and 5, respectively (wherein SEQ ID NOs: 3, 4 and 5 show the heavy chain variable region CDRs 1, 2 and 3, respectively, of PG110). In another embodiment, the anti-NGF antibody comprises a light chain variable region comprising CDRs 1, 2 and 3 having the amino acid sequences of SEQ ID NOs: 6, 7 and 8, respectively (wherein SEQ ID NOs: 6, 7 and 8 show the light chain variable region CDRs 1, 2 and 3, respectively, of PG110). In still another embodiment, the anti-NGF antibody comprises a heavy chain variable region comprising CDRs 1, 2 and 3 having the amino acid sequences of SEQ ID NOs: 3, 4 and 5, respectively, and comprises a light chain variable region comprising CDRs 1, 2 and 3 having the amino acid sequences of SEQ ID NOs: 6, 7 and 8, respectively.

In still other embodiments, the antibody, or antigen-binding portion thereof, has one or more of the following functional properties: a) binds to human NGF but does not bind to human brain-derived neurotrophic factor (BDNF), human neurotrophin 3 (NT-3) or human neurotrophin 4 (NT-4); b) binds to human or rat NGF with a K_{D} of 100 pM or less; c) inhibits binding of NGF to TrkA or p75^{NTR}; d) inhibits NGF-dependent proliferation of TF-1 cells; e) inhibits NGF-dependent chick dorsal root ganglion survival; f) inhibits NGF-dependent PC12 cell neurite outgrowth.

In still other embodiments, the antibody, or antigen-binding portion thereof, is selected from the group consisting of a monoclonal antibody, a human antibody, a humanized antibody, a chimerical antibody, a CDR-grafted antibody, a Fab, a Fab′, a F(ab′)2, a Fv, a disulfide linked Fv, a scFv, a single domain antibody, a diabody, a multispecific antibody, a dual specific antibody, a bispecific antibody, or an antibody in which the potential T cell epitopes have been eliminated. In a further embodiment, the antibody, or antigen-binding portion thereof, is humanized.

In a particularly preferred embodiment, the invention provides compositions containing an anti-NGF antibody that has the combined advantageous features of an extended terminal elimination half life and a prolonged duration of pain alleviation. Accordingly, the invention also provides an anti-NGF antibody comprising a human IgG4 constant region, wherein the human IgG4 constant region comprises a mutation (preferably a hinge region mutation), wherein the antibody has a terminal elimination half-life in a cynomolgus monkey of at least 15 days, more preferably of at least 21 days, and wherein the antibody alleviates pain for a duration of at least about one week to about twelve weeks after administration of a single dose the antibody to a subject

The invention also relates to methods for inhibiting NGF activity in a human subject suffering from an NGF related disease or condition by administering to the human subject a pharmaceutical composition of the invention. In other embodiments, a second pharmaceutical agent, as describe herein, is administered to the subject. In certain embodiments, the NGF related disease or condition is pain. Non-limiting examples of NGF-related diseases and conditions include inflammatory pain, post-operative pain, neuropathic pain, fracture pain, gout joint pain, post-herpetic neuralgia, cancer pain, osteoarthritis or rheumatoid arthritis pain, sciatica, pains associated with sickle cell crises, headaches, dysmenorrhea, endometriosis, musculoskeletal pain, chronic low back pain, fibromyalgia, sprains, visceral pain, ovarian cysts, prostatitis, cystitis, interstitial cystitis, incisional pain, migraine, trigeminal neuralgia, pain from burns and/or wounds, pain associated with trauma, pain associated with musculoskeletal diseases, ankylosing spondilitis, periarticular pathologies, pain from bone metastases, pain from HIV, erythromelalgia or pain caused by pancreatitis or kidney stones. Other examples of NGF-related diseases and conditions include malignant melanoma, Sjogren's syndrome and asthma, such as uncontrolled asthma with severe airway hyper-responsiveness, and intractable cough. Particularly preferred diseases and conditions for treatment according to the methods of the invention include inflammatory pain (particularly osteoarthritis or rheumatoid arthritis pain), musculoskeletal pain (particularly chronic low back pain), neuropathic pain (particularly diabetic neuropathy), cancer pain and pain from bone metastases, interstitial cystitis/painful bladder syndrome, pain associated with chronic abacterial prostatitis, pain associated with endometriosis and/or uterine fibroids and post-operative pain. Preferably, the pain is selected from the group consisting of osteoarthritis pain, chronic low back pain, diabetic neuropathic pain, cancer pain, pain from bone metastases, interstitial cystitis, painful bladder syndrome, pain associated with chronic abacterial prostatitis, pain associated with endometriosis, pain associated with uterine fibroids and post-operative pain.

The pharmaceutical composition of the invention can be administered, for example, intravenously, subcutaneously (e.g., via an injection pen or subcutaneous implant), intramuscularly or intra-articularly, although other suitable routes of administration are described herein.

Kits or articles of manufacture comprising a pharmaceutical composition of the invention are also provided herein.

### BRIEF DESCRIPTION OF THE DRAWINGS

FIG. 1 is a graphic comparison of the stability of Formulation 1 and Formulation 2 over time.

### DETAILED DESCRIPTION OF THE INVENTION

The present invention pertains to improved compositions (e.g., pharmaceutical compositions) of anti-NGF antibodies, or antigen-binding portions thereof, having improved stability. The compositions of the present invention generally comprise an anti-NGF antibody, or antigen-binding fragment thereof, a suitable buffer (e.g., a histidine buffer), a suitable excipient (e.g., polysorbate 80), and having a pH of about 5.0 to about 6.0. The compositions of the present invention may be liquid, suitable for lyophilization, and/or lyophilized.

In order that the present invention may be more readily understood, certain terms are first defined. Additional definitions are set forth throughout the detailed description.

### I. DEFINITIONS

The articles “a” and “an” are used herein to refer to one or to more than one (i.e., to at least one) of the grammatical object of the article. By way of example, “an element” means one element or more than one element.

The term “pharmaceutical formulation” refers to a preparation that is in such form as to permit the biological activity of the active ingredient(s) to be unequivocally effective, and that contains no additional components that are significantly toxic to the subjects to which the formulation would be administered.

The phrase “pharmaceutically acceptable carrier” is art recognized to include a pharmaceutically acceptable material, composition or vehicle, suitable for administration to mammals such as humans. Such carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the composition and not injurious to, or impacting the safety of, the human subject.

“Buffer” refers to a buffered solution that resists changes in pH by the action of its acid-base conjugate components. The buffers of the invention have a pH in the range from about 4 to about 8. Examples of buffers that will control pH in this range include phosphate, acetate (e.g., sodium acetate), succinate (e.g., sodium succinate), gluconate, glutamate, histidine, citrate, and other organic acid buffers.

The term “excipient” refers to an agent that may be added to a composition to provide a desired consistency, e.g., by altering the bulk properties, to improve stability, and/or to adjust osmolality. Examples of commonly used excipients include, but are not limited to, sugars, polyols, amino acids, surfactants, and polymers. “Pharmaceutically acceptable excipients” (e.g., vehicles, additives) are those that can reasonably be administered to a mammalian subject, e.g., human, to provide an effective dose of the active ingredient employed.

As used herein, a “polyol” is a substance with multiple hydroxyl groups, and includes sugar alcohols and sugar acids. Particular polyols have a molecular weight that is less than about 600 D (e.g., in the range from about 120 to about 400 D). Non-limiting examples of polyols include fructose, mannose, maltose, lactose, arabinose, xylose, ribose, rhamnose, galactose, glucose, sorbose, melezitose, raffinose, mannitol, xylitol, erythritol, threitol, sorbitol, glycerol, L-gluconate and metallic salts thereof.

A “sugar” is a carbohydrate with a characteristically sweet taste. Sugars may be classified as monosaccharides, disaccharides, and polysaccharides. “Mono saccharides” are the simple sugars, e.g., fructose, levulose, glucose, and dextrose, or grape sugar. “Disaccharides” include lactose or milk sugar, maltose or malt sugar, crystalline disaccharide, sucrose, and trehalose (also known as mycose or tremalose). Upon hydrolysis, a disaccharide molecule yields two monosaccharide molecules. “Polysaccharides” include such substances as cellulose, dextrin, glycogen, and starch. Polysaccharides are polymeric compounds made up of the simple sugars and can be hydrolyzed to yield simple sugars. The disaccharides are sometimes grouped with the simpler polysaccharides (usually those made up of three or four simple sugar units) to form a class of carbohydrates called “oligosaccharides”.

A “sugar” may also be classified as a “reducing sugar” or a “non-reducing sugar”. The reducing sugars are distinguished by the fact that because of their free, or potentially free, aldehyde or ketone groups they possess the property of readily reducing alkaline solutions of many metallic salts, such as those of copper, silver, bismuth, mercury, and iron. The reducing sugars include, e.g., maltose, lactose, cellobiose, gentiobiose, melibiose, and turanose. Non-limiting examples of nonreducing sugars include sucrose, trehalose, raffinose, melezitose, stachyose, and verbascose.

The term “surfactant” generally includes those agents that protect a protein in a composition from air/solution interface-induced stresses and solution/surface induced-stresses. For example, a surfactant may protect the protein from aggregation. Suitable surfactants may include, e.g., polysorbates, polyoxyethylene alkyl ethers such as Brij 35®; or poloxamers, such as Tween 20, Tween 80, or poloxamer 188. Preferred detergents are polyoxyethylene alkyl ethers, e.g., Brij 35®, Cremophor A25, Sympatens ALM/230; polysorbates/Tweens, e.g., Polysorbate 20, Polysorbate 80, Mirj, and Poloxamers, e.g., Poloxamer 188, Poloxamer 407 and Tweens, e.g., Tween 20 and Tween 80.

A “stable” composition is one in which the active ingredient, e.g., an antibody, therein essentially retains its physical stability and/or chemical stability and/or biological activity during the manufacturing process and/or upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones (1993) Adv. Drug Delivery Rev. 10:29-90.

An antibody “retains its physical stability” in a pharmaceutical composition if it shows substantially no signs of, e.g., aggregation, precipitation and/or denaturation upon visual examination of color and/or clarity, or as measured by UV light scattering or by size exclusion chromatography. Aggregation is a process whereby individual protein molecules or complexes associate covalently or non-covalently to form aggregates. Aggregation can proceed to the extent that a visible precipitate is formed. The physical stability of a pharmaceutical composition containing an anti-NGF antibody may be determined, for example, according to the percentage of monomer protein in the solution, with a low percentage (e.g., less than 3%) of degraded (e.g., fragmented) and/or aggregated protein indicating that the composition is stable.

An antibody “retains its chemical stability” in a pharmaceutical composition of the invention, if the chemical stability at a given time is such that the antibody is considered to still retain its biological activity as defined below. Chemical stability can be assessed by, e.g., detecting and quantifying chemically altered forms of the antibody. Chemical alteration may involve size modification (e.g., clipping) that can be evaluated using size exclusion chromatography, SDS-PAGE and/or matrix-assisted laser desorption ionization/time-of-flight mass spectrometry (MALDI/TOF MS). Other types of chemical alteration include charge alteration (e.g., occurring as a result of deamidation or oxidation), which can be evaluated by, for example, ion-exchange chromatography.

An antibody “retains its biological activity” in a pharmaceutical composition of the invention, if the antibody in a pharmaceutical composition is biologically active for its intended purpose. For example, biological activity is retained if the biological activity of the antibody in the pharmaceutical composition is within about 30%, about 20%, or about 10% (within the errors of the assay) of the biological activity exhibited at the time the pharmaceutical composition was prepared (e.g., as determined in an antigen binding assay). Biological activities of the anti-NGF antibodies contained within the formulations of the invention include, but are not limited to, binding to human NGF, inhibiting binding of NGF to TrkA or p75^{NTR}, inhibiting NGF-dependent proliferation of TF-1 cells, inhibiting NGF-dependent survival and differentiation of neurons and inhibiting NGF-dependent pain transduction. The term “activity” further includes activities such as the binding specificity/affinity of an antibody for an antigen, for example, an anti-NGF antibody that binds to an NGF antigen.

The term “inhibition” as used herein, refers to any statistically significant decrease in biological activity, including full blocking of the activity. For example, “inhibition” can refer to a decrease of about 10%, 20%, 30%, 40%, 50%, 60%, 70%, 80%, 90%, or 100% in biological activity.

The terms “nerve growth factor” or “NGF” are used interchangeably herein and includes variants, isoforms, homologs, orthologs and paralogs. For example, an antibody specific for human NGF may, in certain cases, cross-react with NGF from species other than human. In other embodiments, an antibody specific for human NGF may be completely specific for human NGF and may not exhibit species or other types of cross-reactivity. The term “human NGF” refers to human sequence NGF, such as comprising the amino acid sequence of human NGF-β chain, the precursor form of which has Genbank accession number NP_{—}002497, encoded by the nucleotide sequence of Genbank accession number NM_{—}002506. The human NGF-β chain sequence may differ from human NGF-β of Genbank Accession No. NP_{—}002497 by having, for example, conserved substitutions or substitutions in non-conserved regions wherein the human NGF-β has substantially the same biological function as the human NGF-β of Genbank Accession No. NP_{—}002497. The term “rat NGF” refers to rat sequence NGF, such as comprising the amino acid sequence of rat NGF-β chain, the precursor form of which has Genbank accession number XP_{—}227525, encoded by the nucleotide sequence of Genbank accession number XP_{—}227525. The term “mouse NGF” refers to rat sequence NGF, such as comprising the amino acid sequence of mouse NGF-β chain, the precursor form of which has Genbank accession number NP_{—}038637, encoded by the nucleotide sequence of Genbank accession number NM_{—}013609.

The term “TrkA receptor”, as used herein, refers to an NGF receptor also known in the art as tropomyosin kinase receptor A and neurotrophic tyrosine kinase receptor type 1 (NTRK1). Exemplary, non-limiting sequences for human TrkA receptor include the amino acid sequences of Genbank accession number NP_{—}001012331 (isoform 1), NP_{—}002520 (isoform 2) and NP_{—}001007793 (isoform 3).

The term “p75^{NTR} receptor”, as used herein refers to a neurotrophin receptor, with a molecular weight of approximately 75 kDa, that binds NGF and other neurotrophins, which receptor is described in, e.g., Bothwell, M. (1996) Science 272:506-507. An exemplary, non-limiting sequence for human p75^{NTR} receptor is the amino acid sequence of Genbank accession number NP_{—}002498, encoded by the nucleotide sequence of Genbank accession number NM_{—}002507.

The term “terminal elimination half life”, as used herein with regard to the anti-NGF antibodies, refers to the amount of time needed for the concentration of the antibody, as measured in the serum of a subject to which the antibody has been administered, to be reduced by half once both absorption and redistribution of the antibody are complete. When a group of subjects is used, the geometric mean of the terminal elimination half life in the subjects can be used as the measure of the terminal elimination half life of the antibody.

The term “pharmacologic half life”, as used herein with regard to the anti-NGF antibodies, refers to the average amount of time to maintain drug effect in vivo (MRT for drug effect). It can be calculated as the ratio of area of the first moment baseline-corrected effect-time curve (AUMEC) vs. accumulated baseline-corrected drug effect over time (area under the effect-time curve, AUEC), using the following formula:

$$Pharmacologic Half \text{-} life = \frac{A U M E C}{A U E C} = \frac{\int E ( t ) t d t}{\int E ( t ) d t}$$

When a group of subjects is used, the geometric mean of the pharmacologic half life in the subjects can be used as the measure of the pharmacologic half life of the antibody.

The term “inhibition” as used herein, refers to any statistically significant decrease in biological activity, including full blocking of the activity. For example, “inhibition” can refer to a decrease of about 10%, 20%, 30%, 40%, 50%, 60%, 70%, 80%, 90%, or 100% in biological activity.

The term “antibody” or “immunoglobulin,” as used interchangeably herein, includes whole antibodies and any antigen binding fragment (i.e., “antigen-binding portion”) or single chains thereof that retains the ability to specifically bind to an antigen (e.g., NGF). In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “antigen-binding portion” or “antigen-binding fragment” of an antibody (or simply “antibody portion”) refers to one or more fragments of an antibody that retain the ability to specifically bind to an antigen (e.g., NGF). Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546, Winter et al., PCT publication WO 90/05144 A1), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426 and Huston et al. (1988) Proc. Natl. Acad. Sci. USA 85:5879-5883). Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). Such single chain antibodies are also intended to be encompassed within the term “antigen-binding portion” of an antibody as is well known in the art (Kontermann and Dubel eds., Antibody Engineering (2001) Springer-Verlag. New York, 790 (ISBN 3-540-41354-5).

The term “hinge region mutation”, as used herein, refers to a mutation, such as a point mutation, substitution, addition or deletion, in the hinge region of an immunoglobulin constant domain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies can be prepared using any art recognized technique, for example, a hybridoma method, as described by Kohler et al. (1975) Nature, 256:495, a transgenic animal, as described by, for example, (see e.g., Lonberg, et al. (1994) Nature 368(6474): 856-859), recombinant DNA methods (see, e.g., U.S. Pat. No. 4,816,567), or using phage antibody libraries using the techniques described in, for example, Clarkson et al., Nature, 352:624-628 (1991) and Marks et al., J. Mol. Biol., 222:581-597 (1991). Monoclonal antibodies include chimeric antibodies, human antibodies and humanized antibodies and may occur naturally or be recombinantly produced.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The term “humanized antibody” or “humanized immunoglobulin” refers to an antibody or immunoglobulin that includes at least one humanized antibody or immunoglobulin chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

In an embodiment, the term “humanized antibody” is an antibody or a variant, derivative, analog or fragment thereof which immuno specifically binds to an antigen of interest and which comprises a framework (FR) region having substantially the amino acid sequence of a human antibody and complementary determining regions (CDRs) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′) 2, FabC, Fv) in which all or substantially all of the CDR regions correspond to those of a non-human immunoglobulin (i.e., donor antibody) and all or substantially all of the FR regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In an embodiment, a humanized antibody only contains a humanized light chain. In another embodiment, a humanized antibody only contains a humanized heavy chain. In a particular embodiment, a humanized antibody only contains a humanized variable domain of a light chain and/or humanized heavy chain. The humanized antibody can be selected from any class of immunoglobulins, including IgM, IgG, IgD, IgA and IgE, and any isotype, including without limitation IgG1, IgG2, IgG3 and IgG4. The humanized antibody may comprise sequences from more than one class or isotype, and particular constant domains may be selected to optimize desired effector functions using techniques well known in the art.

The term “epitope” includes any x determinant (e.g., polypeptide) capable of specific binding to an immunoglobulin. In certain embodiments, epitope determinants include chemically active surface groupings of molecules such as amino acids, sugar side chains, phosphoryls, sulfonyls, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “human antibody,” as used herein, is intended to include antibodies having variable regions in which both the framework and CDR regions are derived from human germline immunoglobulin sequences as described, for example, by Kabat et al. (See Kabat, et al. (1991) Sequences of proteins of Immunological Interest, Fifth Edition, U.S. Department of Health and Human Services, NIH Publication No. 91-3242). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

An “isolated antibody,” as used herein, is intended to refer to an antibody which is substantially free of other antibodies having different antigenic specificities (e.g., an isolated antibody that specifically binds to NGF is substantially free of antibodies that specifically bind antigens other than NGF). In addition, an isolated antibody is typically substantially free of other cellular material and/or chemicals.

As used herein, the terms “specific binding,” “specifically binds,” “selective binding,” and “selectively binds,” mean that an antibody or antigen-binding portion thereof, exhibits appreciable affinity for a particular antigen or epitope and, generally, does not exhibit significant cross-reactivity with other antigens and epitopes. “Appreciable” or preferred binding includes binding with an affinity of at least 10⁶, 10⁷, 10⁸, 10⁹ M⁻¹, or 10¹⁰ M⁻¹. Affinities greater than 10⁷M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable proteinaceous entity). Specific or selective binding can be determined according to any art-recognized means for determining such binding, including, for example, according to Scatchard analysis and/or competitive binding assays.

The term “K_{D},” as used herein, is intended to refer to the dissociation equilibrium constant of a particular antibody-antigen interaction or the affinity of an antibody for an antigen, for example, obtained in a titration measurement at equilibrium, or by dividing the dissociation rate constant (Koff) by the association rate constant (Kon). The association rate constant (Kon), the dissociation rate constant (Koff), and the equilibrium dissociation constant (K are used to represent the binding affinity of an antibody to an antigen. Methods for determining association and dissociation rate constants are well known in the art. Fluorescence-based techniques offer high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.) can also be used.

In one embodiment, the antibody according to the present invention binds an antigen (e.g., NGF) with an affinity (K_{D}) of about 100 pM or less (i.e., or better) (e.g., about 90 pM or about 80 pM or about 70 pM or about 60 pM or about 50 pM or about 40 pM or about 30 pM), as measured using a surface plasmon resonance assay or a cell binding assay. In a preferred embodiment, the antibody binds NGF with an affinity (K_{D}) in a range of about 25-35 pM.

The terms “Kₐₛₛ”, “Kₐ” and “Kₒₙ”, as used herein, are intended to refer to the association rate constant for the association of an antibody into the antibody/antigen complex. This value indicates the binding rate of an antibody to its target antigen or the rate of complex formation between an antibody and antigen as is shown by the equation below:

Antibody (“Ab”)+Antigen (“Ag”)→Ab-Ag

The terms “K_{diss}”, “K_{d}” and “K_{off}”, as used herein, are intended to refer to the dissociation rate constant for the dissociation of an antibody from the antibody/antigen complex. This value indicates the dissociation rate of an antibody from its target antigen or separation of Ab-Ag complex over time into free antibody and antigen as shown by the equation below:

Ab+Ag←Ab-Ag

The term “IC₅₀”, as used herein, refers to the concentration of an antibody that inhibits a response, either in an in vitro or an in vivo assay, to a level that is 50% of the maximal inhibitory response, i.e., halfway between the maximal inhibitory response and the untreated response.

The terms “treat,” “treating,” and “treatment,” as used herein, refer to therapeutic or preventative measures described herein. The methods of “treatment” employ administration, to a subject, of an antibody of the present invention, for example, a subject having an NGF-related disease or condition, in order to prevent, cure, delay, reduce the severity of, or ameliorate one or more symptoms of the disease or condition.

The term “NGF-related disease or condition”, as used herein, refers to diseases and conditions in which NGF activity is involved with, or associated with, or mediates or promotes one or more symptoms of the disease or condition.

As used herein, the term “subject” includes any human or non-human animal. In a particular embodiment, the subject is a human. The term “non-human animal” includes all vertebrates, e.g., mammals and non-mammals, such as non-human primates, sheep, dog, cow, chickens, amphibians, reptiles, etc.

As used herein, the term “rebound effect” refers to diminished efficacy of NGF sequestering agents, such as an anti-NGF antibody, occurring in a subject after an initial period of effectiveness after single or repeat administration. For example, treatment with an anti-NGF antibody may initially relieve pain, e.g. due to inflammation or nerve damage or other ethiology, which is then followed by a period of diminished analgesic efficacy in which pain eventually becomes about as intense or more intense than before treatment. In another example, an anti-NGF antibody may exhibit an initial effectiveness in a subject for a period of time after single or repeat administration, such as a period of one week after administration (e.g., days 1-7 after administration), which is then followed by a period of diminished efficacy, such as for a period from 1-2 weeks after administration (e.g., days 7-14 after administration). This “rebound” period may be followed by a period of recovery of efficacy of the anti-NGF antibody. For example, there can be a biphasic profile of analgesia after single or repeat administration of an anti-NGF antibody, with an intermediate period of reduced efficacy or even exaggerated pain sensation. This rebound effect can be assessed in, for example, clinical pain studies, experimental models of pain and/or other models of anti-NGF efficacy. This rebound effect can be associated with, for example, increased pain in the subject and/or increased adverse events (such as abnormal sensations, ranging from allodynia to dysesthesia, paresthesia and hyper- or hypoesthesia) during the rebound period. Although not intending to be limited by mechanism, the rebound effect may be caused by altered NGF expression, altered TrkA or p75 receptor expression or signaling or any other mechanism that results in transient diminished efficacy after single or repeat administration of an anti-NGF after an initial period of efficacy.

Various aspects of the invention are described in further detail in the following subsections.

### II. PHARMACEUTICAL COMPOSITIONS OF THE INVENTION

The present invention provides liquid and lyophilized pharmaceutical compositions comprising an anti-NGF antibody or antigen binding fragment thereof, having improved properties as compared to art-recognized compositions. The compositions of the invention are able to maintain solubility and stability of the anti-NGF antibody or antigen binding fragment thereof, e.g., during manufacturing, storage, and/or repeated freeze/thaw processing steps or extended exposure to increased air-liquid interfaces (e.g., do not show significant opalescence, aggregation, or precipitation). For example, the compositions of the invention maintain a low level of protein aggregation (i.e., less than 3%), despite containing high amounts (e.g., about 10 to about 240 mg/mL), of the antibody or antigen binding fragment thereof. The compositions of the invention also maintain a low viscosity within ranges suitable for subcutaneous injection, despite containing high amounts (e.g., about 10 to about 240 mg/mL), of the antibody. Furthermore, the compositions of the invention maintain solubility, maintain a low viscosity suitable for subcutaneous or intravenous injection, and maintain stability over a pH range of, e.g., about pH 5.0 to about pH 6.0. Thus, the antibody compositions of the invention overcome a number of known challenges for antibody compositions, including stability, viscosity, turbidity, and physical degradation challenges.

Accordingly, in one aspect, the pharmaceutical compositions comprise an anti-NGF antibody or antigen binding fragment thereof, a buffer and an excipient which are sufficient to maintain stability of the anti-NGF antibody or antigen binding fragment thereof in liquid and/or lyophilized form.

Anti-NGF antibodies, and antigen-binding fragments thereof, that can be used in the compositions of the invention and methods of making such antibodies, and antigen-binding fragments thereof, are described in detail herein. The amount of the antibody present in the composition is determined, for example, by taking into account the desired dose, volume(s) and mode(s) of administration. In certain embodiments of the invention, the compositions of the invention, e.g., liquid and/or lyophilized compositions (upon reconstitution) comprise a protein concentration of about 10 to about 240 mg/mL, about 20 to about 120 mg/mL, about 40 to about 240 mg/mL, about 50-150 mg/mL, about 15 to about 75 mg/ml, or about 10 to about 20 mg/ml of the anti-human NGF antibodies, or antigen-binding fragments thereof. Although the preferred embodiments of the invention are compositions comprising high protein concentrations, it is also contemplated that the compositions of the invention may comprise an antibody concentration between about 1 mg/mL and about 240 mg/mL, between about 1 mg/ml and about 150 mg/ml or between about 50 mg/mL and about 150 mg/mL is between about 30 mg/mL and about 50 mg/mL. In one embodiment of the invention, the concentration of the antibody is about 100 mg/mL. In one embodiment of the invention, the concentration of the antibody is about 60 mg/mL. In one embodiment of the invention, the concentration of the antibody is about 30 mg/mL. In another embodiment, the concentration of the antibody is about 20 mg/mL. In another embodiment, the concentration of the antibody is about 10 mg/mL. In another embodiment of the invention, the compositions, comprise a concentration of the antibody of about 55 mg/mL.

Ranges intermediate, e.g., to the above-recited ranges, e.g., 75-90 mg/ml, are also intended to be part of this invention. For example, ranges of values using a combination of any of the above-recited values as upper and/or lower limits are intended to be included. In addition, concentrations of anti-NGF antibody intermediate to the above recited amounts and concentrations are also intended to be part of this invention (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or about 240 mg/mL).

In some embodiments of the invention, the compositions, e.g., lyophilized compositions, comprise about 1-100 mg, 1-75 mg, 1-55 mg, 1-30 mg, 1-20 mg, 1-10 mg, 10-20 mg, 15-75 mg, 100-150 mg, 110-150 mg, 100-140 mg, 110-140 mg, 120-140 mg, 130-140 mg of the anti-NGF antibody. In other embodiments, the compositions, e.g., lyophilized compositions, comprise about 40-240, 40-200, 40-180, 40-160, 40-140, 40-120 mg, 45-100 mg, 50-80 mg, or 55-70 mg of the antibody. In one embodiment, the compositions, e.g., lyophilized compositions, comprise about 10 mg of the antibody. In another embodiment, the compositions, e.g., lyophilized compositions, comprise about 20 mg of the antibody.

Ranges intermediate to the above-recited ranges, e.g., 132-138, or 55-65, are also intended to be part of this invention. For example, ranges of values using a combination of any of the above-recited values as upper and/or lower limits are intended to be included. In addition, amounts and concentrations of anti-NGF antibody intermediate to the above recited amounts and concentrations are also intended to be part of this invention (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 134.1, 134.2, 134.3, 134.4, 134.5, 134.6, 134.7, 134.8, 134.9, 135, 136, 137, 138, 139, 140, 141, 142, 143, 144, 145, 146, 147, 148, 149, or about 150 mg of the antibody).

Buffers used in the pharmaceutical compositions of the invention are those suitable to maintaining the pH of the composition in a range from about 4.0 to about 8.0, from about 5.0 to about 7.0, from about 5.0 to about 6.5, from about 5.5 to about 7.0. Preferably, the buffer maintains the pH of the pharmaceutical compositing of the invention in the range from about 5.0 to about 6.0, from about 6.0 to about 7.0, from about 5.5 to about 6.0, from about 6.0 to about 6.5, from about 5.75 to about 6.25 and from about 5.25 to about 5.75. In one embodiment, the pH of the compositions of the invention is about 6.0. In one embodiment, the pH of the compositions of the invention is about 5.5. In one embodiment, the pH of the compositions of the invention is about 5.0. Ranges and values intermediate to the above-recited pHs are also intended to be part of this invention (e.g., pHs of 5.0, 5.1, 5.2, 5.3, 5.4, 5.5, 5.6, 5.7, 5.8, 5.9, 6.0, 6.1, 6.2, 6.3, or 6.4). Ranges of values using a combination of any of the above-recited values as upper and/or lower limits are intended to be included.

Examples of buffers that will control the pH within the range of about 5.5 to about 7.0 include phosphate, acetate (e.g., sodium acetate), succinate (e.g., sodium succinate), arginine, gluconate, glutamate, histidine, citrate and other organic acid buffers.

In one embodiment, the buffer is histidine. In certain embodiments of the invention, the concentration of the histidine in the composition is about 1-100 mM, about 1-30 mM, about 5-30 mM, about 10-30 mM, about 30-60 mM, about 30-40 mM, about 10-50 mM, about 15-60 mM, about 15-45 mM, about 15-30 mM, about 15-25 or about 15-20 mM. In one embodiment, the concentration of the histidine in the composition is about 20 mM. In another embodiment, the concentration of the histidine in the composition is about 15 mM. In another embodiment, the concentration of the histidine in the composition is about 30 mM. Concentrations and ranges of histidine intermediate to the above recited concentrations are also intended to be part of this invention (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or about 100 mM of histidine). Ranges of concentrations using a combination of any of the above-recited values as upper and/or lower limits are intended to be included.

In other embodiments of the invention, the compositions, e.g., lyophilized compositions, comprise about 1-10 mg of histidine, or about 2-5 mg histidine. In one embodiment, the compositions comprise about 6 mg, e.g., about 5.7 mg, of histidine. In one embodiment, the compositions comprise about 5 mg, e.g., about 4.7 mg, of histidine. In one embodiment, the compositions comprise about 2-3 mg of histidine. Amounts and ranges of histidine intermediate to the above-recited amounts are also intended to be part of this invention (e.g., about 1, 1.5, 2, 2.2, 2.3, 2.5, 3, 3.5, 4, 4.5 5, 5.1, 5.2, 5.3, 5.4, 5.5, 5.6, 5.7, 5.8, 5.9, 6, 6.5, 7, 7.5 8, 8.5, 9, 9.5, or about 10 mg of histidine). Ranges of amounts using a combination of any of the above-recited values as upper and/or lower limits are intended to be included.

A detergent or surfactant may also be added to the antibody compositions of the invention as an excipient. Exemplary detergents include nonionic detergents such as polysorbates (e.g., polysorbates 20, 80, etc.) or poloxamers (e.g., poloxamer 188). The amount of detergent added is such that it reduces aggregation of the formulated antibody and/or minimizes the formation of particulates in the composition and/or reduces adsorption. Suitable surfactants may include, e.g., polysorbates, polyoxyethylene alkyl ethers such as Brij 35®; or poloxamers, such as Tween 20, Tween 80, or poloxamer 188. Preferred detergents are polyoxyethylene alkyl ethers, e.g., Brij 35®, Cremophor A25, Sympatens ALM/230; polysorbates/Tweens, e.g., Polysorbate 20, Polysorbate 80, Mirj, and Poloxamers, e.g., Poloxamer 188, Poloxamer 407 and Tweens, e.g., Tween 20 and Tween 80.

In a preferred embodiment of the invention, the composition includes a surfactant which is a polysorbate. In another preferred embodiment of the invention, the composition contains the detergent polysorbate 80. In one embodiment, the composition contains between about 0.01 and about 2.0 mg/mL, about 0.01 to about 1 mg/mL, about 0.05 to about 2.0 mg/mL, about 0.05 to about 1.0 mg/mL, about 0.05 to about 0.5 mg/mL, about 0.05 to about 0.1 mg/mL of polysorbate 80. In one embodiment, the composition comprises about 1 mg/mL of polysorbate 80. In another embodiment, the composition comprises about 0.1 mg/mL of polysorbate 80. In yet another embodiment, the composition comprises about 0.05 mg/mL of polysorbate 80. In one embodiment, the composition comprises between about 0.001% and about 0.1%, between about 0.005% and about 0.08%, between about 0.007% and about 0.06%, between about 0.01% and about 0.04%, between about 0.01% and about 0.03%, or between about 0.01% and 0.02% polysorbate 80. In one embodiment, the composition comprises about 0.01% polysorbate 80. In one embodiment, the composition comprises about 0.02% polysorbate 80. In other embodiments of the invention, however, the compositions are essentially free of or do not contain a surfactant, such as Tween or polysorbate.

In certain embodiments of the invention, the compositions, e.g., lyophilized compositions, may comprise between about 0.01 and 0.5 mg, e.g., about 0.05, 0.1, 0.15, 0.2, 0.25, 0.3, 0.35, 0.4, 0.45 or about 0.5 mg of a surfactant. In one embodiment, the compositions, e.g., lyophilized compositions, comprise about 0.20 mg of a surfactant, e.g., polysorbate 80. In one embodiment, the compositions e.g., lyophilized compositions, comprise about 0.10 mg of a surfactant, e.g., polysorbate 80. Ranges and amounts intermediate to the above-recited concentrations and amounts of surfactants are also intended to be part of this invention, e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, and 2.0 mg/mL of a surfactant. In addition, ranges of values using a combination of any of the above-recited values as upper and/or lower limits are intended to be included, e.g., 0.04 to 1.8 mg/mL of a surfactant.

The compositions of the invention may also comprise a polyol. Polyols useful in the compositions of the invention include, but are not limited to, one or more of trehalose, fructose, mannose, maltose, lactose, arabinose, xylose, ribose, rhamnose, galactose, glucose, sorbose, melezitose, raffinose, mannitol, xylitol, erythritol, threitol, sorbitol, glycerol, L-gluconate and metallic salts thereof.

In one embodiment, the polyol is selected from the group consisting of sorbitol, glycerol, trehalose and mannitol or combinations thereof. In one embodiment, the polyol is not mannitol. In certain embodiments, the concentration of the polyol in the compositions of the invention is about 1 to about 100 mg/mL, about 10 to about 90 mg/mL, about 20 to about 80 mg/mL, about 30 to about 70 mg/mL, about 40 to about 60 mg/mL, or about 50 to about 60 mg/mL. In other embodiments, the compositions, e.g., lyophilized compositions, of the invention comprise a polyol at a concentration of about 10-100 mg, about 10 to about 90 mg/mL, about 20 to about 80 mg/mL, about 30 to about 70 mg/mL, about 40 to about 60 mg/mL, or about 50 to about 60 mg/mL. In other embodiments, the compositions of the invention, e.g., compositions suitable for lyophilization, comprise about 1-50 mg/mL, about 10-30 mg/mL or about 20-25 mg/mL of a polyol.

In still other embodiments, the compositions of the invention, e.g., lyophilized compositions, comprise about 10-120, about, about 20-120, about 30-120, about 40-120, about 50-120, about 60-120, about 10-110, about 10-100, about 10-90, about 10-80, about 10-70 mg of a polyol or combination thereof. Concentrations and ranges of polyols intermediate to the above recited concentrations are also intended to be part of this invention (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or about 100 mg/mL of polyol). Ranges of concentrations of polyols using a combination of any of the above-recited values as upper and/or lower limits are intended to be included, e.g., 35-70 mg/ml of polyol.

In one embodiment, a suitable polyol for use in the compositions of the invention is a sugar alcohol, e.g., sorbitol. The compositions of the invention may comprise about 20-60 mg/mL, about 30-60 mg/mL, about 20-50 mg/mL, or about 35-45 mg/mL of sorbitol. In one embodiment, the compositions comprise about 40 mg/mL of sorbitol.

In another embodiment, a suitable polyol for use in the compositions of the invention is mannitol. The compositions of the invention may comprise about 1-50 mg/mL, about 10-40 mg/mL, about 20-30 mg/mL, about 20-25 mg/mL of mannitol. In one embodiment, the compositions comprise about 20 mg/mL of mannitol. In one embodiment, the compositions of the invention, e.g., compositions suitable for lyophilization, comprise about 1-50 mg/mL, about 10-30 mg/mL or about 20-25 mg/mL of mannitol, and preferably comprise 20 mg/mL mannitol. In yet other embodiments, the compositions of the invention, e.g., lyophilized compositions, comprise about 40-60 mg, about 45-55 mg, or about 48-52 mg of mannitol. In one embodiment, the compositions comprise about 50 mg, e.g., about 49.5 mg, of mannitol.

In another embodiment, a suitable polyol for use in the compositions of the invention is glycerol. The compositions of the invention may comprise about 1-50 mg/mL, about 10-40 mg/mL, about 20-30 mg/mL, about 20-25 mg/mL, or about 20 mg/mL of glycerol.

One or more sugars may also be added to the compositions of the invention. Non-limiting examples of sugars that are useful in the compositions of the invention include maltose, lactose, cellobiose, gentiobiose, melibiose, and turanose, fructose, levulose, glucose, and dextrose, lactose, sucrose, and trehalose (also known as mycose or tremalose), raffinose, melezitose, stachyose, and verbascose. In certain embodiments, the concentration of sugar is about 1 to about 120 mg/ml, about 1 to about 100 mg/mL, about 10 to about 90 mg/mL, about 20 to about 80 mg/mL, about 30 to about 70 mg/mL, about 40 to about 60 mg/mL, or about 50 to about 60 mg/mL. In other embodiments, the compositions of the invention, e.g., lyophilized compositions, comprise about 10-120, about 20-120, about 30-120, about 40-120, about 50-120, about 60-120, about 10-110, about 10-100, about 10-90, about 10-80, about 10-70 mg of a sugar.

In certain embodiments, the sugar is sucrose and is present in the compositions of the invention at about 10-100 mg/mL, about 10-90 mg/mL, about 10-80 mg/mL, about 10-70 mg/mL, about 20-90 mg/mL, about 20-80 mg/mL, about 20-70 mg/mL, about 30-70 mg/mL, or about 25-65 mg/mL of sucrose. In one embodiment, the compositions comprise about 70 mg/mL of sucrose. In one embodiment, the compositions, e.g., compositions suitable for lyophilization, comprise about 5 mg/mL of sucrose. In one embodiment, the compositions, e.g., compositions suitable for lyophilization, comprise about 45 mg/mL of sucrose. In another embodiment, the compositions, e.g., compositions suitable for lyophilization, comprise about 46 mg/mL of sucrose.

In yet other embodiments, the compositions of the invention, e.g., lyophilized compositions, comprise about 1-100, about 1-70, about 1-50, about 10-120 mg, about 10-100 mg, about 10-50 mg, about 10-20 mg, or about 12 mg, e.g., about 12.25 mg, of sucrose. In one embodiment, the compositions, e.g., lyophilized compositions, comprise about 50-120 mg, about 75-120 mg or about 100-120 mg of sucrose, e.g., about 110, 11, 112, 113, 114, 115, 116, 117, 118, 119 or 120 mg of sucrose. In one embodiment, the compositions, e.g., lyophilized compositions, comprise about 113 mg, of sucrose. In another embodiment, the compositions, e.g., lyophilized compositions, comprise about 70 mg, of sucrose. In another embodiment, the compositions, e.g., lyophilized compositions, comprise about 20 mg, of sucrose. In another embodiment, the compositions, e.g., lyophilized compositions, comprise about 10 mg, of sucrose. In another embodiment, the compositions, e.g., lyophilized compositions, comprise about 5 mg, of sucrose.

In another embodiment, the sugar is trehalose. Trehalose may be present in the compositions at about 10-100 mg/mL, about 10-90 mg/mL, about 10-80 mg/mL, about 10-70 mg/mL, about 20-90 mg/mL, about 20-80 mg/mL, about 20-70 mg/mL, about 30-70 mg/mL, about 25-65 mg/mL, or about 35-55 mg/ml. In one embodiment, the compositions, e.g., compositions suitable for lyophilization, comprise about 40-50 mg/ml, e.g., about 45 mg/mL of trehalose.

Concentrations and ranges of sugars intermediate to the above recited concentrations are also intended to be part of this invention (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or about 120 mg/mL of sugars). Ranges of concentrations of sugars using a combination of any of the above-recited values as upper and/or lower limits are intended to be included, e.g., 35-70 mg/ml of sugars.

In other embodiments, any combination of one or more of the foregoing sugars and one or more of the foregoing polyols may be included together in a composition of the invention. For example, a composition of the invention, e.g., a composition suitable for lyophilization, may comprise a polyol, e.g., mannitol, and a sugar, e.g., sucrose. In certain embodiments, the molar ratio of the anti-NGF antibody, or antigen binding fragment thereof, to polyol (e.g., mannitol), sugar (e.g., sucrose) or combinations thereof (e.g., mannitol and sucrose) is greater than about 1:1200, preferably greater than about 1:1400, more preferably between about 1:1400 and 1:1500, or greater than about 1:1500.

In certain embodiments of the invention, the compositions further comprise an amino acid, e.g., methionine. In one embodiment, the compositions comprise about 1-10 mM, about 2-10 mM, about 2-9 mM, about 2-8 mM, about 2-7 mM, about 2-6 mM, about 2-5 mM, about 3-8 mM, about 3-7 mM, about 3-6 mM, or about 3-5 mM of methionine. In one embodiment, the compositions comprise about 4 mM methionine. In another embodiment, the compositions comprise about 5 mM methionine. In one embodiment, the compositions comprising methionine also comprise a polyol, e.g., mannitol and/or a sugar, e.g., sucrose. In one embodiment, the compositions comprise methionine, mannitol and sucrose. In one embodiment, the compositions do not comprise an amino acid, e.g., methionine.

In certain embodiments of the invention, the compositions, e.g., lyophilized compositions, may comprise between about 0.1-10 mg, 0.5-9 mg, 1.0-8 mg, 1-6 mg, 1-5 mg, 1-4 mg, 1-3 mg or 1-2 mg, e.g., about 1.5, 1.6, 1.7, 1.75, 1.8, 1.81, 1.82, 1.83, 1.84, 1.85, 1.9, or 2.0 mg of methionine. In one embodiment, the compositions, e.g., lyophilized compositions, comprise about 1.8 mg, e.g., 1.83 mg, of methionine. Ranges and amounts intermediate to the above-recited concentrations and amounts of methionine are also intended to be part of this invention, e.g., 1, 1.5, 2, 2.5, 3, 3.5, 4, 4.5, 5, 5.5, 6, 6.5, 7, 7.5, 8, 8.5, 9, 9.5, and 10 mM. In addition, ranges of values using a combination of any of the above-recited values as upper and/or lower limits are intended to be included, e.g., 3.5-9 mM.

In one embodiment, the composition is essentially free of preservatives, such as benzyl alcohol, phenol, m-cresol, chlorobutanol and benzethonium Cl. In another embodiment, a preservative may be included in the composition. One or more other pharmaceutically acceptable carriers, excipients or stabilizers such as those described in Remington's Pharmaceutical Sciences 16th edition, Osol, A. Ed. (1980) may be included in the composition provided that they do not significantly adversely affect the desired characteristics of the composition. Acceptable carriers, excipients or stabilizers are nontoxic to recipients at the dosages and concentrations employed and include; additional buffering agents; co-solvents; antioxidants including ascorbic acid and methionine; chelating agents such as EDTA; metal complexes (e.g., Zn-protein complexes); biodegradable polymers such as polyesters; and/or salt-forming counterions such as sodium.

In particular embodiments, the pharmaceutical compositions of the invention are formulated as a liquid either comprising, consisting essentially of, or consisting of (a) about 1-10, 5-15, 10-20, 10-30, 20-50 or 20-75 mg/mL of an anti-NGF antibody, or antigen binding fragment thereof; (b) about 5-50, 5-30, 5-20 or 10-20 mM histidine; and (c) about 0.01-0.02% polysorbate 80; wherein the pH of the composition is about 5.0-6.0 or about 5.5. In certain preferred embodiments, the anti-NGF antibody is PG100 or an antigen binding fragment of PG110. In certain preferred embodiments, the concentration of histidine is about 10 or 15 mM.

In particular embodiments, the pharmaceutical compositions of the invention are formulated as a liquid either comprising, consisting essentially of, or consisting of (a) about 1-10, 5-15, 10-20, 10-30, 20-50 or 20-75 mg/mL of an anti-NGF antibody, or antigen binding fragment thereof; (b) about 5-50, 5-30, 5-20 or 10-20 mM histidine; (c) about 0.01-0.2% polysorbate 80; and (d) about 20-80, 30-80 or 40-80 mg of a polylol; wherein the pH of the composition is about 5.0-6.0 or about 5.5. In certain preferred embodiments, the anti-NGF antibody is PG100 or an antigen binding fragment of PG110. In certain preferred embodiments, the concentration of histidine is about 10 or 15 mM. In certain preferred embodiments, the polylol is mannitol or sorbitol. In other preferred embodiments, the concentration of polylol is 20, 30 or 40 mg/mL. In other preferred embodiments, the composition further comprises a sugar, preferably sucrose or trehalose, at about 10-20, 20-50 or 30-80 mg/mL.

In particular embodiments, the pharmaceutical compositions of the invention are formulated as a liquid either comprising, consisting essentially of, or consisting of (a) about 1-10, 5-15, 10-20, 10-30, 20-50 or 20-75 mg/mL of an anti-NGF antibody, or antigen binding fragment thereof; (b) about 5-50, 5-30, 5-20 or 10-20 mM histidine; (c) about 0.01-0.2% polysorbate 80; and (d) about 20-80, 30-80 or 40-80 mg/mL of a sugar; wherein the pH of the composition is about 5.0-6.0 or about 5.5. In certain preferred embodiments, the anti-NGF antibody is PG100 or an antigen binding fragment of PG110. In certain preferred embodiments, the concentration of histidine is about 10 or 15 mM. In certain preferred embodiments, the sugar is sucrose or trehalose. In other preferred embodiments, the concentration of sugar is 70 or 80 mg/mL. In particular embodiments, the pharmaceutical compositions of the invention are provided in lyophilized form suitable for reconsititution to liquid form. For each mL of reconsitituted liquid, the lyophilized compositions comprise, consist essentially of, or consist of (a) about 1-10, 5-15, 10-20, 10-30, 20-50 or 20-75 mg of an anti-NGF antibody, or antigen binding fragment thereof; (b) about 1-20, 1-10, 1-5 or 2-4 mg histidine; and (c) about 0.1 to 0.2 mg polysorbate 80. In certain preferred embodiments, the compositions contain about 10, 20 or 50 mg of PG100 or an antigen binding fragment of PG110. In certain preferred embodiments, the composition contains about 2-3 mg histidine. In certain preferred embodiments the composition contains 0.1 mg polysorbate 80.

In particular embodiments, the pharmaceutical compositions of the invention are provided in lyophilized form suitable for reconsititution to liquid form. For each mL of reconsitituted liquid, the lyophilized compositions comprise, consist essentially of, or consist of (a) about 1-10, 5-15, 10-20, 10-30, 20-50 or 20-75 mg of an anti-NGF antibody, or antigen binding fragment thereof; (b) about 1-20, 1-10, 1-5 or 2-4 mg histidine; (c) about 0.1 to 0.2 mg polysorbate 80; and (d) about 20-80, 30-80 or 40-80 mg of a polylol. In certain preferred embodiments, the compositions contain about 10, or 50 mg of PG100 or an antigen binding fragment of PG110. In certain preferred embodiments, the composition contains about 2-3 mg histidine. In certain preferred embodiments the composition contains 0.1 mg polysorbate 80. In certain preferred embodiments, the composition contains 10, 20, 30 or 40 mg mannitol or sorbitol. In certain preferred embodiments, the composition further contains about 10-40 mg of a sugar, preferably sucrose or trehalose.

In particular embodiments, the pharmaceutical compositions of the invention are provided in lyophilized form suitable for reconsititution to liquid form. For each mL of reconsitituted liquid, the lyophilized compositions comprise, consist essentially of, or consist of (a) about 1-10, 5-15, 10-20, 10-30, 20-50 or 20-75 mg of an anti-NGF antibody, or antigen binding fragment thereof; (b) about 1-20, 1-10, 1-5 or 2-4 mg histidine; (c) about 0.1 to 0.2 mg polysorbate 80; and (d) 20-80, 30-80 or 40-80 mg/mL of a sugar. In certain preferred embodiments, the compositions contain about 10, 20 or 50 mg of PG100 or an antigen binding fragment of PG110. In certain preferred embodiments, the composition contains about 2-3 mg histidine. In certain preferred embodiments the composition contains 0.1 mg polysorbate 80. In certain preferred embodiments, the composition contains about 20, 40, 70 or 80 mg of a sugar, preferably sucrose or trehalose.

The compositions of the invention may also be combined with one or more other therapeutic agents as necessary for the particular indication being treated, preferably those with complementary activities that do not adversely affect the antibody of the composition. Such therapeutic agents are suitably present in combination in amounts that are effective for the purpose intended.

The compositions to be used for in vivo administration must be sterile. This is readily accomplished by filtration through sterile filtration membranes prior to, or following, preparation of the composition.

As described above, the compositions of the invention, e.g., liquid, suitable for lyophilization and lyophilized compositions, have advantageous stability and storage properties. Stability of the liquid composition is not dependent on the form of storage, and includes, but is not limited to, compositions which are frozen, lyophilized, spray-dried, or compositions in which the active ingredient is suspended. Stability can be measured at a selected temperature for a selected time period. In one aspect of the invention, the protein in the liquid compositions is stable in a liquid form for at least about 3 months; at least about 4 months, at least about 5 months; at least about 6 months; at least about 12 months; at least about 18 months or longer. Ranges intermediate to the above recited time periods are also intended to be part of this invention, e.g., about 9 months, and so forth. In addition, ranges of values using a combination of any of the above-recited values as upper and/or lower limits are intended to be included.

Preferably, the composition is stable at room temperature, or at about 30° C., or at 40° C. for at least about 1 month and/or stable at about 2-8° C. for at least about 1 year, or more preferably stable at about 2-8° C. for at least about 2 years. Furthermore, the composition is preferably stable following freezing (to, e.g., −80° C.) and thawing of the composition, hereinafter referred to as a “freeze/thaw cycle.” In one embodiment, the composition is stable following one, two, three or more freeze-thaw cycles.

Stability of a protein in a liquid composition may also be defined as the percentage of monomer, aggregate, or fragment, or combinations thereof, of the protein in the composition, for example, as measured by UV light scattering or by size exclusion chromatography. In one aspect of the invention, a stable liquid composition is a composition having less than about 10%, and preferably less than about 5% and more preferably less than about 2% of the protein being present as aggregate in the composition.

In one embodiment, the physical stability of a liquid composition is determined by determining turbidity of the composition following a stir stress assay, e.g., 24 hour or 48-hour stir-stress assay. For example, a stir stress assay may be performed by placing a suitable volume of a liquid composition in a beaker with a magnetic stirrer, e.g., (multipoint HP, 550 rpm), removing aliquots at any suitable time, e.g., at T0-T48 (hrs), and performing suitable assays as desired on the aliquots. Samples of a composition under the same conditions but without stirring serve as control. Turbidity measurements may be performed using a laboratory turbidity measurement system from Hach (Germany) and are reported as nephelometric units (NTU).

The compositions of the invention also have advantageous tolerability properties. Tolerability is evaluated based on assessment of subject-perceived injection site pain using the Pain Visual Analog Scale (VAS). A (VAS) is a measurement instrument that measures pain as it ranges across a continuum of values, e.g., from none to an extreme amount of pain. Operationally a VAS is a horizontal line, about 100 mm in length, anchored by numerical and/or word descriptors, e.g., 0 or 10, or ‘no pain’ or ‘excruciating pain’, optionally with additional word or numeric descriptors between the extremes, e.g., mild, moderate, and severe; or 1 through 9) (see, e.g., Lee et al. (2000) Acad. Emerg. Med. 7:550).

Additional indicators of tolerability that may be measured include, for example, the Draize Scale (hemorrhage, petechiae, erythema, edema, pruritus) and bruising.

### III. ANTI-NGF ANTIBODIES

Anti-NGF antibodies that may be used in the pharmaceutical compositions of the invention are described, for example, in PCT Publication No. WO/2010/128398, PCT Publication No. WO 2001/78698, PCT Publication No. WO 2001/64247, PCT Publication No. WO 2002/096458, PCT Publication No. WO 2004/032870, PCT Publication No. WO 2004/058184, PCT Publication No. WO 2005/061540, PCT Publication No. WO 2005/019266, PCT Publication No. WO 2006/077441, PCT Publication No. WO 2006/131951, PCT Publication No. WO 2006/110883, PCT Publication No. WO 2009/023540, U.S. Pat. No. 7,449,616; U.S. Publication No. US 20050074821, U.S. Publication No. US 20080033157, U.S. Publication No. US 20080182978 or U.S. Publication No. US 20090041717, the entire contents of each of which are hereby incorporated herein by reference, particularly, the contents as relating to anti-NGF antibodies.

In one embodiment, the anti-NGF antibodies to be used in the pharmaceutical compositions are characterized by having enhanced in vivo stability, as evidenced by the long terminal elimination half life observed in vivo. Although not intending to be limited by mechanism, it is thought that the extended terminal elimination half life of the antibody results from a reduced clearance rate of the antibody rather than from an increase in the distribution volume of the antibody. Preferably, the antibodies to be used in the pharmaceutical compositions of the invention comprise a human IgG4 constant region that comprises a mutation. A preferred mutation is a hinge region mutation. Preferably, the hinge region mutation comprises mutation of serine at amino acid position 108 of SEQ ID NO: 9 (wherein SEQ ID NO: 9 shows the amino acid sequence of the wild-type human IgG4 constant region). More preferably, the hinge region mutation comprises mutation of the serine at amino acid position 108 of SEQ ID NO: 9 to proline. In a preferred embodiment, the human IgG4 constant region comprises the amino acid sequence of SEQ ID NO: 10.

In one embodiment, an anti-NGF antibody to be used in the pharmaceutical compositions of the invention exhibits an unexpectedly long terminal elimination half life, such as a terminal elimination half life in a cynomolgus monkey of at least 15 days and typically in the range of about 15 to about 22 days (or in a range of 15-22 days), or in a range of about 15 days to about 28 days (or in a range of 15-28 days) or in a range of about 21 days to about 28 days (or in a range of 21-28 days). This stabilized anti-NGF antibody also exhibits a terminal elimination half life in rats of at least 8 days, typically in the range of about 8 to about 9 days (or in a range of 8-9 days).

In one embodiment, a preferred anti-NGF antibody for use in pharmaceutical compositions of the invention, PG110, exhibits a mean terminal elimination half life in cynomolgus monkeys of at least 15 days and typically longer. For example, in one cynomolgus monkey study, a mean terminal elimination half life in a range of about 15 to about 22 days was observed. In another cynomolgus monkey study, a mean terminal elimination half life in a range of about 21 to about 28 days was observed. Furthermore, PG110 exhibits a mean terminal elimination half life in rats of about 8 to about 9 days. Still further, as it is known in the art that the terminal elimination half life of IgG in humans is about twice that of monkeys, it is predicted that the anti-NGF antibodies of the invention, such as PG110, will have terminal elimination half life in humans of at least 10-30 days, or at least 10 days, or at least 15 days, or at least 20 days, or at least 25 days, or more preferably at least 30 days or at least 40 days, or in a range of about 10 days to about 40 days (or in range of 10-40 days) or in a range of about 15 to about 30 days (or in a range of 15-30 days). Additionally or alternatively, the antibody may exhibit a mean pharmacologic half life in humans of at least 30 days, or at least 35 days, or at least 40 days, or in a range of at least four to six weeks (or in a range of four to six weeks), or in a range of at least four to seven weeks (or in a range of four to seven weeks) or in a range of at least four to eight weeks (or in a range of four to eight weeks). As described further in Example 8, an anti-NGF antibody of the invention of the invention has been shown to have a mean pharmacologic half life in humans in the aforementioned ranges.

The terminal elimination half life for PG110 in cynomolgus monkeys is considerably longer than the half life that has been reported in the art for other IgG4 antibodies in cynomolgus monkeys. For example, a half life of about 40-90 hours (about 1.6-3.8 days) in cynomolgus monkeys has been reported for CDP571, an IgG4 anti-TNF antibody (see Stephens, S. et al. (1995) Immunol. 85:668-674). Similarly, a half life of about 3 days in cynomolgus monkeys has been reported for natalizumab, an IgG4 anti-integrin antibody (see Refusal CHMP Assessment Report for Natalizumab, European Medicines Agency, London, 15 Nov. 2007, Doc. Ref. EMEA/CHMP/8203/2008).

In one embodiment, the pharmaceutical compositions of the invention comprise anti-NGF antibodies wherein the preferred hinge region mutation is a serine to proline mutation at position 108 in SEQ ID NO: 9. This mutation has been previously described in the art (see Angal, S. et al. (1993) Mol. Immunol. 30:105-108) and reported to abolish the heterogeneity of IgG4 molecules, in particular the formation of half antibodies containing a single heavy chain and a single light chain. Accordingly, substitution of an amino acid other than proline at position 108 of SEQ ID NO: 9 also is encompassed by the invention, wherein the substitution achieves the same effect as the Ser to Pro mutation in eliminating the heterogeneity of the IgG4 molecule (e.g., the formation of half antibodies). The ability of a mutation at position 108 to eliminate the heterogeneity of the IgG4 molecule can be assessed as described in Angal et al. (1993), supra.

In addition to, or alternative to, the modification at position 108 of SEQ ID NO: 9, other IgG hinge region mutations have been described that improve the affinity of the FcRn-IgG interaction, resulting in an extended half life for the modified IgG. Examples of such additional or alternative modifications include mutations at one or more IgG constant region residues corresponding to: Thr250, Met252, Ser254, Thr256, Thr307, Glu308, Met428, His433 and/or Asn434 (as described further in Shields, R. L. et al. (2001) J. Biol. Chem. 276:6591-6604; Petkova, S. B. et al. (2006) Int. Immunol. 18:1759-1769; Hinton, P. R. et al. (2004) J. Biol. Chem. 279:6213-6216; Kamei, D. T. et al. (2005) Biotechnol. Bioeng. 92:748-760; Vaccaro, C. et al. (2005) Nature Biotechnol. 23:1283-1288; Hinton, P. R. et al. (2006) J. Immunol. 176:346-356).

Still further, alternative to hinge region mutations, other stabilizing modifications of the IgG4 constant region have been described. For example, in other embodiments, the mutation of the human IgG4 constant region comprises substitution of the IgG4 CH3 region with an IgG1 CH3 region, substitution of the IgG4 CH2 and CH3 regions with the IgG1 CH2 and CH3 regions or substitution of the arginine at position 409 of the IgG4 constant region (according to Kabat numbering) with a lysine, as described further in U.S. Patent Publication 20080063635. In yet other embodiments, the mutation of the human IgG4 constant region comprises substitution of Arg409, Phe405 or Lys370 (according to Kabat numbering), such as substitution of Arg409 with Lys, Ala, Thr, Met or Leu, or substitution of Phe405 with Ala, Val, Gly or Leu, as described further in PCT Publication WO 2008/145142.

A desired mutation can be introduced into the human IgG4 constant region domain using standard recombinant DNA techniques, such as site-directed mutagenesis or PCR-mediated mutagenesis of a nucleic acid encoding the human IgG4 constant region. Furthermore, DNA encoding an antibody heavy chain variable region can be introduced into an expression vector encoding a mutated human IgG4 constant region such that the variable region and constant region become operatively linked, to thereby create vector encoding a full-length immunoglobulin heavy chain in which the constant region is a mutated human IgG4 constant region. The expression vector then can be used to express the full-length immunoglobulin heavy chain using standard recombinant protein expression methods. For example, an anti-NGF antibody of the invention can be constructed as described in further detail in Example 1.

The terminal elimination half life of an antibody can be determined using standard methods known in the art. For example, after administration of the antibody to a subject (e.g., a cynomolgus monkey, a Sprague-Dawley rat), blood samples can be obtained at various time points after administration and the concentration of antibody in the serum from the blood samples can be determined using a technique known in the art for determining antibody concentration (such as an ELISA assay). Calculation of the terminal half life of the antibody can be accomplished using known pharmacokinetic methods, for example using a computer system and software designed to calculate pharmacokinetic parameters (a non-limiting example of which is the SNBL USA Pharmacokinetics Analysis System with WinNonlin software).

In one embodiment, the pharmaceutical compositions of the invention contain an anti-NGF antibody, or antigen-binding portion thereof, comprising the heavy and light chain variable regions of the PG110 antibody. The heavy chain variable region of PG110 is shown in SEQ ID NO: 1 and the light chain variable region of PG110 is shown in SEQ ID NO: 2. Accordingly, in one embodiment, the anti-NGF antibody of the invention comprises a heavy chain variable region comprising the amino acid sequence of SEQ ID NO: 1. In another embodiment, the anti-NGF antibody of the invention comprises a light chain variable region comprising the amino acid sequence of SEQ ID NO: 2. In yet another embodiment, the anti-NGF antibody of the invention comprises a heavy chain variable region comprising the amino acid sequence of SEQ ID NO: 1 and a light chain variable region comprising the amino acid sequence of SEQ ID NO: 2.

The full-length amino acid sequence of the PG110 heavy chain (variable and constant regions) is shown in SEQ ID NO: 13. This heavy chain can be prepared from a precursor heavy chain, which includes a leader or signal sequence, such as the amino acid sequence shown in SEQ ID NO: 12. The precursor heavy chain of SEQ ID NO: 12 is encoded by the nucleotide sequence shown in SEQ ID NO: 11.

The full-length amino acid sequence of the PG110 light chain (variable and constant regions) is shown in SEQ ID NO: 16. This light chain can be prepared from a precursor light chain, which includes a leader or signal sequence, such as the amino acid sequence shown in SEQ ID NO: 15. The precursor light chain of SEQ ID NO: 15 is encoded by the nucleotide sequence shown in SEQ ID NO: 14.

Accordingly, in another embodiment, the anti-NGF antibody for use in the pharmaceutical compositions of the invention comprises a heavy chain comprising the amino acid sequence of SEQ ID NO: 13, wherein the antibody has a serum half-life in a cynomolgus monkey of at least 15 days. In another embodiment, the serum half-life in a cynomolgus monkey can be in a range of about 15 days to about 22 days (or in a range of 15-22 days). In other embodiments, the serum half-life in a rat can be at least 8 days or in a range of about 8 days to about 9 days (or in a range of 8-9 days). In yet other embodiments, the serum half-life in a human can be at least 10-30 days, or at least 10 days, or at least 15 days, or at least 20 days, or at least 25 days, or at least 30 days or at least 40 days or in a range of about 10 days to about 40 days (or in a range of 10-40 days) or in a range of about 15 to about 30 days (or in a range of 15-30 days). Additionally or alternatively, the antibody may exhibit a mean pharmacologic half life in humans of at least 30 days, or at least 35 days, or at least 40 days, or in a range of at least four to six weeks (or in a range of four to six weeks), or in a range of at least four to seven weeks (or in a range of four to seven weeks) or in a range of at least four to eight weeks (or in a range of four to eight weeks). Preferably, the heavy chain is encoded by the nucleotide sequence of SEQ ID NO: 11. Preferably, the light chain of the antibody comprises the amino acid sequence of SEQ ID NO: 16. Preferably, the light chain is encoded by the nucleotide sequence of SEQ ID NO: 14.

In yet another embodiment, the anti-NGF antibody for use in the pharmaceutical compositions of the invention comprises a heavy chain comprising the amino acid sequence of SEQ ID NO: 13 and a light chain comprising the amino acid sequence of SEQ ID NO: 16.

In yet another embodiment, the anti-NGF antibody for use in the pharmaceutical compositions of the invention comprises a heavy chain encoded by the nucleotide sequence of SEQ ID NO: 11. and a light chain encoded by the nucleotide sequence of SEQ ID NO: 14.

Given that the binding specificity of PG110 is provided by the complementarity determining regions (CDRs) of the variable domain, in another embodiment, the anti-NGF antibody for use in the pharmaceutical compositions of the invention comprises the CDRs of the heavy chain of PG110, the light chain of PG110 or both. The heavy chain CDRs 1, 2 and 3 of PG110 are shown in SEQ ID NOs: 3, 4 and 5, respectively. The light chain CDRs 1, 2 and 3 of PG110 are shown in SEQ ID NOs: 6, 7 and 8, respectively. Accordingly, in one embodiment, the anti-NGF antibody of the invention comprises a heavy chain variable region comprising CDRs 1, 2 and 3 having the amino acid sequences of SEQ ID NOs: 3, 4 and 5, respectively. In another embodiment, the anti-NGF antibody for use in the pharmaceutical compositions of the invention comprises a light chain variable region comprising CDRs 1, 2 and 3 having the amino acid sequences of SEQ ID NOs: 6, 7 and 8, respectively. In yet another embodiment, the anti-NGF antibody for use in the pharmaceutical compositions of the invention comprises a heavy chain variable region comprising CDRs 1, 2 and 3 having the amino acid sequences of SEQ ID NOs: 3, 4 and 5, respectively, and comprises a light chain variable region comprising CDRs 1, 2 and 3 having the amino acid sequences of SEQ ID NOs: 6, 7 and 8, respectively.

In yet another embodiment, an anti-NGF antibody for use in the pharmaceutical compositions of the invention can comprise heavy and light chain variable regions comprising amino acid sequences that are homologous to the heavy and/or light chain variable regions of PG110, and wherein the antibodies retain the enhanced in vivo stability exhibited by PG110. For example, the heavy chain variable region of the anti-NGF antibody can comprise an amino acid sequence that is at least 90% homologous, more preferably at least 95% homologous, more preferably at least 97% homologous and even more preferably at least 99% homologous to the amino acid sequence of SEQ ID NO: 1. The light chain variable region of the anti-NGF antibody can comprise an amino acid sequence that is at least 90% homologous, more preferably at least 95% homologous, more preferably at least 97% homologous and even more preferably at least 99% homologous to the amino acid sequence of SEQ ID NO: 2.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm. For example, the percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

In yet another embodiment, an anti-NGF antibody for use in the pharmaceutical compositions of the invention can comprise heavy and light chain variable regions comprising the amino acid sequences of the heavy and/or light chain variable regions of PG110 but wherein one or more conservative substitutions have been introduced into the sequence(s) yet the antibody retains the enhanced in vivo stability exhibited by PG110. For example, the heavy chain variable region of the anti-NGF antibody can comprise an amino acid sequence that is identical to the amino acid sequence of SEQ ID NO: 1 except for 1, 2, 3, 4 or 5 conservative amino acid substitutions as compared to SEQ ID NO: 1. The light chain variable region of the anti-NGF antibody can comprise an amino acid sequence that is identical to the amino acid sequence of SEQ ID NO: 2 except for 1, 2, 3, 4 or 5 conservative amino acid substitutions as compared to SEQ ID NO: 2.

As used herein, the term “conservative amino acid substitution” is intended to refer to amino acid modifications that do not significantly affect or alter the binding or stability characteristics of the antibody containing the amino acid sequence. Such conservative modifications include amino acid substitutions, additions and deletions. Modifications can be introduced into an antibody of this disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the variable regions of PG110 can be replaced with other amino acid residues from the same side chain family and the altered antibody can be tested for retained function using the functional assays described herein.

In yet another embodiment, an anti-NGF antibody for use in the pharmaceutical compositions of the invention comprises antigen-binding regions (i.e., variable regions) that bind to the same epitope on NGF as the PG110 antibody or that cross-compete for binding to NGF with PG110. Accordingly, in one embodiment, the anti-NGF antibody of the invention competes for binding to NGF with an antibody comprising a heavy chain variable region comprising the amino acid sequence of SEQ ID NO: 1 and a light chain variable region comprising the amino acid sequence of SEQ ID NO: 2.

Such cross-competing antibodies can be identified based on their ability to cross-compete with PG110 in standard NGF binding assays. For example, standard ELISA assays can be used in which a recombinant NGF protein (e.g., human NGF-β) is immobilized on the plate, one of the antibodies is fluorescently labeled and the ability of non-labeled antibodies to compete off the binding of the labeled antibody is evaluated. Additionally or alternatively, BIAcore analysis can be used to assess the ability of the antibodies to cross-compete. Suitable binding assays that can be used to test the ability of an antibody to compete for binding to NGF with an antibody comprising a heavy chain variable region comprising the amino acid sequence of SEQ ID NO: 1 and a light chain variable region comprising the amino acid sequence of SEQ ID NO: 2, have been described previously (e.g., WO/2010/128398).

In still other embodiments, an anti-NGF antibody useful in the compositions of the invention exhibits one or more functional properties of the PG110 antibody. For example, an anti-NGF antibody of the invention can exhibit one or more of the following functional properties:
binds to human NGF but does not bind to human brain-derived neurotrophic factor (BDNF), human neurotrophin 3 (NT-3) or human neurotrophin 4 (NT-4);
binds to human or rat NGF with a K_{D} of 100 pM or less;
inhibits binding of NGF to TrkA or p75^{NTR};
inhibits NGF-dependent proliferation of TF-1 cells;
inhibits NGF-dependent chick dorsal root ganglion survival;
inhibits NGF-dependent PC12 cell neurite outgrowth.
   These functional properties can be assessed using the in vitro assays known in the art and described in, for example, WO/2010/128398. With respect to the specific binding of the antibody to human NGF, as used herein the term “does not bind to brain-derived neurotrophic factor (BDNF), human neurotrophin 3 (NT-3) or human neurotrophin 4 (NT-4)” is intended to mean that the amount of observed binding of the antibody to BDNF, NT-3 or NT-4, in a standard binding assay (e.g., ELISA, or other suitable in vitro assay as described in the Examples) is comparable to background levels of binding (e.g., for a control antibody), for example no more than 2-fold above background levels, or less than 5% binding to BDNF, NT-3 or NT-4 as compared to binding to human NGF (wherein the level of binding to human NGF is set as 100% binding).

In yet another embodiment, the anti-nerve growth factor (NGF) antibody for use in the pharmaceutical compositions of the invention comprises a human IgG4 constant region, wherein the human IgG4 constant region comprises the amino acid sequence of SEQ ID NO: 10 (or wherein the human IgG4 constant region comprises a mutation of serine at amino acid position 108 of SEQ ID NO: 9, preferably a serine to proline mutation at position 108), and wherein the antibody binds to human or rat NGF with a K_{D} of 100 pM or less (or, alternatively, with a K_{D} of 300 pM or less, 200 pM or less, 150 pM or less, 75 pM or less or 50 pM or less), inhibits binding of NGF to TrkA or p75^{NTR} with an IC₅₀ of 250 pM or less (or, alternatively, with an IC₅₀ of 500 pM or less 400 pM or less, 300 pM or less or 200 pM or less), and inhibits NGF-dependent proliferation of TF-1 cells with an IC₅₀ of 50 ng/ml or less (or, alternatively, with an IC₅₀ of 150 ng/ml or less, 100 ng/ml or less, 75 ng/ml or less or 40 ng/ml or less). Preferably, the antibody has mean terminal elimination half-life in humans of at least 10-30 days, or at least 10 days, or at least 15 days, or at least 20 days, or at least 25 days, or at least 30 days or in a range of about 10 days to about 40 days (or in a range of 10-40 days) or in a range of about 15 days to about 30 days (or in a range of 15-30 days). Additionally or alternatively, the antibody may exhibit a mean pharmacologic half life in humans of at least 30 days, or at least 35 days, or at least 40 days, or in a range of at least four to six weeks (or in a range of four to six weeks), or in a range of at least four to seven weeks (or in a range of four to seven weeks) or in a range of at least four to eight weeks (or in a range of four to eight weeks). Additionally or alternatively, the antibody may exhibit a mean terminal elimination half life in a cynomolgus monkey of at least 15 days and typically in the range of about 15 to about 22 days (or in a range of 15-22 days), or in a range of about 15 days to about 28 days (or in a range of 15-28 days) or in a range of about 21 days to about 28 days (or in a range of 21-28 days). Additionally or alternatively, the antibody may exhibit a terminal elimination half life in rats of at least 8 days, typically in the range of about 8 to about 9 days (or in a range of 8-9 days). The antibody may further exhibit one or more additional functional properties, such as binding to human NGF but not binding to human brain-derived neurotrophic factor (BDNF), human neurotrophin 3 (NT-3) or human neurotrophin 4 (NT-4); inhibiting NGF-dependent chick dorsal root ganglion survival; and/or inhibiting NGF-dependent PC12 cell neurite outgrowth. Preferably, the antibody alleviates pain for a duration of at least about one week to about twelve weeks after administration of a single dose the anti-NGF antibody to a subject. Preferably, the antibody comprises a heavy chain variable region comprising CDRs 1, 2 and 3 having the amino acid sequences of SEQ ID NOs: 3, 4 and 5, respectively, or the antibody comprises a light chain variable region comprising CDRs 1, 2 and 3 having the amino acid sequences of SEQ ID NOs: 6, 7 and 8, respectively, or the antibody comprises a heavy chain variable region comprising CDRs 1, 2 and 3 having the amino acid sequences of SEQ ID NOs: 3, 4 and 5, respectively, and a light chain variable region comprising CDRs 1, 2 and 3 having the amino acid sequences of SEQ ID NOs: 6, 7 and 8, respectively. Preferably, the antibody comprises a heavy chain variable region comprising the amino acid sequence of SEQ ID NO: 1 or the antibody comprises a light chain variable region comprising the amino acid sequence of SEQ ID NO: 2, or the antibody comprises a heavy chain variable region comprising the amino acid sequence of SEQ ID NO: 1 and a light chain variable region comprising the amino acid sequence of SEQ ID NO: 2, or the antibody competes for binding to NGF with an antibody comprising a heavy chain variable region comprising the amino acid sequence of SEQ ID NO: 1 and a light chain variable region comprising the amino acid sequence of SEQ ID NO: 2.

In yet another embodiment, the anti-NGF antibody for use in the pharmaceutical compositions of the invention does not exhibit a rebound effect when administered to a subject (e.g., the antibody is administered at a dosage and at a frequency such that a rebound effect is avoided in the subject). A rebound effect, in which an anti-NGF antibody exhibits diminished efficacy in a subject after an initial period of effectiveness after single or repeat administration, has been reported in both animal models and clinical studies of other anti-NGF antibodies. For example, such an effect, referred to as a “rebound phenomenon”, was reported for an anti-rat NGF antibody in a chronic constriction injury (CCI) model in rats (Ro, L-S. et al. (1999) Pain 79:265-274). Additionally, clinical pain studies with the anti-NGF antibody tanezumab (also known as RN624, E3, CAS Registry No. 880266-57-9) have been reported in which a period of increased adverse events, such as sensitivity to touch and a ‘pins & needles’ sensation, was observed after an initial analgesic period (see presentation by Hefti, Franz F., Rinat Neuroscience, LSUHSC, Shreveport, La., Sep. 26, 2006). Although not intending to be limited by mechanism, it is thought that the prolonged terminal elimination half life of the anti-NGF antibodies described herein allows them to avoid exhibiting a rebound effect. Thus, other advantages of the anti-NGF antibodies used in the compositions of the invention include a more consistent and prolonged activity in vivo as compared to other prior art anti-NGF antibodies. Given the prolonged terminal elimination half life of such anti-NGF antibodies, lower dosages can be used (as compared to other anti-NGF antibodies), and compositions containing the antibody can be used at more frequent intervals if necessary, such that dosage and timing treatment regimens can be chosen such that a rebound effect in the subject is avoided.

In yet another embodiment, the anti-NGF antibody for use in the pharmaceutical compositions of the invention is capable of alleviating pain for a long duration in a subject, for example the antibody is capable of alleviating pain for a duration of at least about one week to about twelve weeks (or for one week to twelve weeks), after administration of a single dose of the anti-NGF antibody to a subject. In one embodiment, the antibody alleviates pain for a duration of at least about one week (or at least one week) after administration of a single dose of the anti-NGF antibody to a subject. In another embodiment, the antibody alleviates pain for a duration of at least about two weeks (or at least two weeks) after administration of a single dose of the anti-NGF antibody to a subject. In another embodiment, the antibody alleviates pain for a duration of at least about four weeks (or at least four weeks) after administration of a single dose of the anti-NGF antibody to a subject. In another embodiment, the antibody alleviates pain for a duration of at least about eight weeks (or at least eight weeks) after administration of a single dose of the anti-NGF antibody to a subject. In another embodiment, the antibody alleviates pain for a duration of at least about twelve weeks (or at least twelve weeks) after administration of a single dose of the anti-NGF antibody to a subject. In another embodiment, the antibody alleviates pain for a duration of at least about four weeks to about twelve weeks (or for four weeks to twelve weeks) after administration of a single dose of the anti-NGF antibody to a subject. In another embodiment, the antibody alleviates pain for a duration of at least about eight weeks to about twelve weeks (or for eight weeks to twelve weeks) after administration of a single dose of the anti-NGF antibody to a subject.

The ability of the antibody to alleviate pain in a subject can be assessed using assays established in the art. Suitable animals models for assessing the duration of pain alleviation by an anti-NGF antibody are described in, for example, PCT Publication No. WO 2006/131951 and U.S. Patent Publication 20080182978. Non-limiting examples of such animal models include a neuropathic pain model evoked by chronic constriction of the sciatic nerve, a post-surgical pain model involving incision of the hind paw, a rheumatoid arthritis pain model involving complete Freund's adjuvant (CFA)-induced arthritis and cancer pain models such as described in Halvorson, K. G. et al. (2005) Cancer Res. 65:9426-9435 and Sevcik, M. A. et al. (2005) Pain 115:128-141. Furthermore, pain alleviation can be evaluated clinically in humans and the duration of pain alleviation can be determined based on pain levels reported by the human subject(s) being treated with the anti-NGF antibody.

In yet other embodiments, an anti-NGF antibody for use in the pharmaceutical compositions of the invention can comprise a heavy chain variable region and/or light chain variable region of an anti-NGF antibody that is prepared by a standard method known in the art for raising monoclonal antibodies, such as the standard somatic cell hybridization technique described by Kohler and Milstein (1975) Nature 256: 495 to create non-human monoclonal antibodies (which antibodies can then be humanized), as well as phage display library techniques or methods using transgenic animals expressing human immunoglobulin genes. Phage display library techniques for selecting antibodies are described in, for example, McCafferty et al., Nature, 348:552-554 (1990). Clarkson et al., Nature, 352:624-628 (1991), Marks et al., J. Mol. Biol., 222:581-597 (1991) and Hoet et al (2005) Nature Biotechnology 23, 344-348; U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al. Methods of using transgenic animals expressing human immunoglobulin genes to raise antibodies are described in, for example, Lonberg, et al. (1994) Nature 368(6474): 856-859; Lonberg, N. and Huszar, D. (1995) Intern. Rev. Immunol. 13: 65-93, Harding, F. and Lonberg, N. (1995) Ann. N.Y. Acad. Sci. 764:536-546; U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; PCT Publication WO 02/43478 to Ishida et al., U.S. Pat. Nos. 5,939,598; 6,075,181; 6,114,598; 6,150,584 and 6,162,963 to Kucherlapati et al.

In various embodiments, an anti-NGF antibody for use in the compositions of the invention can be a chimeric antibody, a humanized antibody or a human antibody. Furthermore, the antibody can be one in which potential T cell epitopes have been eliminated. Methods of eliminating potential T cell epitopes to thereby reduce the potential immunogenicity of an antibody have been described in the art (see e.g., U.S. Patent Publication No. 20030153043 by Carr et al.).

An antibody or antibody portion of the invention can be derivatized or linked to another functional molecule (e.g., another peptide or protein). Accordingly, the antibodies and antibody portions for use in the pharmaceutical compositions of the invention are intended to include derivatized and otherwise modified forms of the PG110 antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

### IV. ANTIBODY PRODUCTION

Anti-NGF antibodies for use in the pharmaceutical compositions of the invention may be produced using nucleic acid molecules that encode the anti-NGF antibodies. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of this disclosure can be, for example, DNA or RNA and may or may not contain intronic sequences. In a preferred embodiment, the nucleic acid is a cDNA molecule. Nucleic acids of this disclosure can be obtained using standard molecular biology techniques.

In one embodiment, an anti-NGF antibody for use in the pharmaceutical compositions of the invention is encoded by a nucleic acid molecule comprising the nucleotide sequence of SEQ ID NO: 11. In another embodiment, an anti-NGF antibody for use in the pharmaceutical compositions of the invention is encoded by a nucleic acid molecule comprising the nucleotide sequence of SEQ ID NO: 14.

Once DNA fragments encoding V_{H} and V_{L} segments are obtained, these DNA fragments can be further manipulated by standard recombinant DNA techniques, for example to convert the variable region genes to full-length antibody chain genes such that the variable region is operatively linked to the constant region (see e.g., Example 1). The term “operatively linked”, as used in this context, is intended to mean that the two DNA fragments are joined such that the amino acid sequences encoded by the two DNA fragments remain in-frame.

Antibodies for use in the pharmaceutical compositions of the invention can be produced in a host cell using methods known in the art (e.g., Morrison, S. (1985) Science 229:1202). For example, to express the antibodies, the DNAs encoding the heavy and light chains can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Additionally, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of typically carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of this disclosure include Chinese Hamster Ovary (CHO cells) (including dhfr⁻ CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. Another preferred expression system is the GS gene expression system disclosed in WO 87/04462 (to Wilson), WO 89/01036 (to Bebbington) and EP 338,841 (to Bebbington). When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

In one embodiment, an anti-NGF antibody for use in the pharmaceutical compositions of the invention is produced using an expression vector, wherein the vector comprises the nucleotide sequence of SEQ ID NO: 11 encoding an antibody heavy chain and the nucleotide sequence of SEQ ID NO: 14 encoding an antibody light chain. A preferred expression vector comprises the GS (glutamine synthetase) gene. In another preferred embodiment, the preferred host cell of the invention is a CHO (Chinese Hamster Ovary) cell.

In yet another preferred embodiment, the anti-NGF antibody for use in the pharmaceutical compositions of the invention is produced by culturing a host cell comprising an expression vector which comprises the nucleotide sequence of SEQ ID NO: 11 (encoding an antibody heavy chain) and the nucleotide sequence of SEQ ID NO: 14 (encoding an antibody light chain) such that an anti-NGF antibody comprising a heavy chain encoded by SEQ ID NO: 11 and a light chain encoded by SEQ ID NO: 14 is expressed.

### V. METHODS OF ADMINISTRATION

A pharmaceutical composition of the present invention can be administered by a variety of methods known in the art. As will be appreciated by the skilled artisan, the route and/or mode of administration will vary depending upon the desired results. Generally, a pharmaceutical composition of the invention is suitable for intravenous, intra-articular, subcutaneous, intramuscular, parenteral, intra-tumoral, intranasal, intravesicular, intrasynovial, oral, mucosal, sublingual, spinal or epidermal administration or by instillation into body cavities (e.g., abdomen, pleural cavity, nasal sinuses). In certain preferred embodiments, the pharmaceutical composition of the invention are suitable for administration intravenously, subcutaneously (e.g., via an injection pen) or intra-articularly.

Pharmaceutical compositions of the invention can be administered alone or in combination therapy, i.e., combined with other agents. For example, the combination therapy can include a composition of the present invention with at least one or more additional pharmaceutical agents. For example, at least one or more additional pharmaceutical agents may be administered separately or can also be incorporated into the compositions. In a preferred embodiment, a pharmaceutical composition of the invention comprising an anti-NGF antibody or antigen binding fragment thereof, is administered in combination with a second pharmaceutical agent, wherein the second pharmaceutical agent is selected from the group consisting of NSAIDs, analgesics (including opioid analgesics and atypical analgesics), local anaesthetics, nerve blocks, phenol blocks, therapeutic antibodies, steroids, anti-convulsants, anti-depressants, topical capsaicin and antiviral agents. A particularly preferred class of second pharmaceutical agents for use in pain alleviation are the opioid analgesics. Additionally or alternatively, a second treatment regimen can be combined with use of an antibody of the invention, for example in the alleviation of pain. Examples of such second treatment regimens include radiotherapy (e.g., for cancer pain), surgical procedures (e.g., gasserian ganglion and retrogasserian ablative (needle) procedures for trigeminal neuralgia), hypnosis and acupuncture.

Examples of NSAIDS include acetylated salicylates including aspirin; nonacetylated salicylates including salsalate, diflunisal; acetic acids including etodolac, diclofenac, indomethacin, ketorolac, nabumetone; propionic acids including fenoprofen, flurbiprofen, ibuprofen, ketoprofen, naproxen, naproxen sodium, oxaprozin; fenamates including meclofenamate, mefenamic acid; phenylbutazone, piroxicam; COX-2 inhibitors including celecoxib, etoricoxib, valdecoxib, rofecoxib, lumiracoxib. Examples of analgesics include paracetamol (acetaminophen), tramadol, tapentadol, capsaicin (topical), opioid analgesics and atypical analgesics. Examples of opioid analgesics include morphine, codeine, thebaine, hydromorphone, hydrocodone, oxycodone, oxymorphone, desomorphine, diacetylmorphine, nicomorphine, dipropanoylmorphine, benzylmorphine, ethylmorphine, fentanyl, pethidine, methadone, tramadol and propoxyphene. Examples of atypical analgesics include trycyclic anti-depressants, carbazepine, gabapentin, pregabalin, duloxetine and caffeine. Examples of steroids include intraarticular corticosteroids (IACs) and prednisone. Examples of therapeutic antibodies include anti-TNF antibodies, such as Remicade® and Humira®, and antiCD20 antibodies, such as Rituxan® and Arzerra™. Examples of antiviral agents include acyclovir and oseltamivir phosphate (Tamiflu®).

In a preferred embodiment, the combination therapy can include an anti-NGF antibody pharmaceutical composition of the present invention with at least one or more TrkA inhibitors (e.g., compounds that antagonize TrkA activity). TrkA inhibitors can function, for example, by interacting extracellularly with the TrkA receptor, or by interacting intracellularly with the TrkA signaling transduction machinery (e.g., inhibition of TrkA kinase activity). Non-limiting examples of extracellular TrkA inhibitors include anti-TrkA antibodies (such as the humanized anti-TrkA antibodies described in US Patent Publication No. 20090208490 and US Patent Publication No. 20090300780) and NGF peptide mimetics that antagonize TrkA (such as described in Debeir, T. et al. (1999) Proc. Natl. Acad. Sci. USA 96:4067-4072). Non-limiting examples of intracellular TrkA inhibitors include cell-penetrating peptides that antagonize TrkA function (e.g., as described in Hirose, M. et al. (2008) J. Pharmacol. Sci. 106:107-113; Ueda, K. et al. (2010) J. Pharmacol. Sci., Mar. 30, 2010 issue) and small molecule inhibitors such as TrkA kinase inhibitors (e.g., as described in Wood, E. R. et al. (2004) Bioorg. Med. Chem. Lett. 14:953-957; Tripathy, R. et al. (2008) Bioorg. Med. Chem. Lett. 18:3551-3555). Other non-limiting examples of TrkA inhibitors include ARRY-470 and ARRY-872 (Array Biopharma).

In another preferred embodiment, the combination therapy can include an anti-NGF antibody composition of the present invention with at least one or more Protein Kinase C (PKC) inhibitors (e.g., compounds that antagonize PKC activity).

Sterile injectable formulations of the pharmaceutical compositions of the invention can be prepared by incorporating the active compound with one or a combination of ingredients (e.g., buffer, excipient, etc.) enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Formulations may conveniently be presented in dosage unit form and may be prepared by any methods known in the art of pharmacy. Dosage unit form as used herein refers to physically discrete units suited as unitary dosages for the subjects to be treated; each unit containing a predetermined quantity of active compound calculated to produce the desired therapeutic effect.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound which is the lowest dose effective to produce a therapeutic effect. Such an effective dose will generally depend upon the factors described above.

In one embodiment, an effective amount of the composition of the present invention is an amount that inhibits NGF activity in a subject suffering from a disorder in which NGF activity is detrimental. In one embodiment, the composition provides an effective dose of 100 mg per injection of the antibody. In another embodiment, the composition provides an effective dose which ranges from about 0.1 to about 100 mg of antibody. If desired, the effective daily dose of the pharmaceutical composition may be administered as two, three, four, five, six or more sub-doses administered separately at appropriate intervals throughout the day, optionally, in unit dosage forms

In one embodiment of the invention, the dosage of the antibody in the composition is between about 5 and about 150 mg. In another embodiment, the dosage of the antibody in the composition is between about 25 and about 100 mg. In another embodiment, the dosage of the antibody in the composition is between about 40 and about 80 mg. In another embodiment, the dosage of the antibody in the composition is between about 50 and about 100 mg. In another embodiment, the dosage of the antibody in the composition is between about 0.1 and about 100 mg, between about 0.5 and 75 mg, between about 1.0 and 60 mg, between about 5 and 40 mg, between about 10 and 30 mg, or between about 10 and 20 mg. The composition is especially suitable for large antibody dosages of more than 10 mg. In a particular embodiment of the invention, the composition provides an antibody at a dose of about 10 mg or about 20 mg. In another embodiment, the composition provides an antibody at a dose of about 80 mg or about 100 mg.

In one embodiment of the invention, the dosage of the antibody in the composition is between about 0.1 to about 150 mg, 1 to about 150 mg, about 5 to about 145 mg, about 10 to about 140 mg, about 15 135 mg, about 20 to about 130 mg, about 25 to about 125 mg, about 30 to about 120 mg, about 35 to about 115 mg, about 40 to about 110 mg, about 45 to about 105 mg, about 50 to about 100 mg, about 55 to about 95 mg, about 60 to about 90 mg, about 65 to about 85 mg, about 70 to about 80 mg, or about 75 mg. In one embodiment, the dosage of the antibody is 10 mg. In one embodiment, the dosage of the antibody is 20 mg. Ranges intermediate to the above recited dosages, e.g., about 2 to about 149 mg are also intended to be part of this invention. For example, ranges of values using a combination of any of the above recited values as upper and/or lower limits are intended to be included.

For particular routes of administration, a suitable delivery device may be chosen for use. For example, for subcutaneous or intramuscular administration, an injection pen (e.g., that can be self-administered) can be used. Such injection pens, also referred to as injectors, are known in the art, including those that contain a liquid dose of antibody (such as that described in PCT publication WO 2008/005315.). Also for subcutaneous administration, a subcutaneous implant can be used. Additionally, transcutaneous delivery can be achieved by use of a topical cutaneous (skin) patch (e.g., adhesive patch). Transcutaneous delivery also can be achieved by injection of dry powder (such as injectors commercially available from Glide Pharma). Still further, for delivery into the lungs (e.g., in the treatment of asthma or intractable cough), pulmonary administration can be employed, e.g., by use of an inhaler or nebulizer, and composition with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244, WO 97/32572, WO 97/44013, WO 98/31346, and WO 99/66903

In a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the pharmaceutical compositions of the invention can be further formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. A typical single dose (which may be administered on a dosing schedule as described further below) might range from about any of 0.1 μg/kg to 1 μg/kg to 3 μg/kg to 30 μg/kg to 300 μg/kg to 3000 μg/kg (3 mg/kg), to 30 mg/kg to 100 mg/kg or more, depending on the factors described herein. For example, an anti-NGF antibody may be administered at about 1 μg/kg, about 10 μg/kg, about 20 μg/kg, about 50 μg/kg, about 100 μg/kg, about 200 μg/kg, about 300 μg/kg, about 400 μg/kg about 500 μg/kg, about 1 mg/kg, about 2 mg/kg or about 3 mg/kg. In a preferred embodiment, the anti-NGF antibody is administered at a dose in a range from about 3 μg/kg to about 3000 μg/kg. In another preferred embodiment, the anti-NGF antibody is administered at a dose of 100 μg/kg. In another preferred embodiment, the anti-NGF antibody is administered at a dose of 200 μg/kg. In another preferred embodiment, the anti-NGF antibody is administered at a dose of 300 μg/kg. In another preferred embodiment, the anti-NGF antibody is administered at a dose of 400 μg/kg.

For repeated administrations over several days, weeks or months or longer, depending on the condition, the treatment is sustained until a desired suppression of symptoms occurs or until sufficient therapeutic levels are achieved (e.g., to reduce pain).

An exemplary dosing regimen comprises administering an initial dose in a range of about 3 μg/kg to 500 μg/kg, followed by a monthly maintenance dose of about 3 μg/kg to 500 μg/kg of the anti-NGF antibody. In another embodiment, a dose of about 200 μg/kg is administered once every month. In yet another embodiment, a dose of about 400 μg/kg is administered once every two months. However, other dosage regimens may be useful, depending on the pattern of pharmacokinetic decay that the practitioner wishes to achieve. For example, in some embodiments, dosing from one to four times a week is contemplated. However, given the long duration of pain alleviation by the anti-NGF antibodies, less frequent dosing may be used. In some embodiments, the anti-NGF antibody is administered once every week, once every 2 weeks, once every 3 weeks, once every 4 weeks, once every 5 weeks, once every 6 weeks, once every 7 weeks, once every 8 weeks, once every 9 weeks, once every 10 weeks, once every 15 weeks, once every 20 weeks, once every 25 weeks, once every 26 weeks, or longer. In some embodiments, the anti-NGF antibody is administered once every 1 month, once every 2 months, once every 3 months, once every 4 months, once every 5 months, once every 6 months, or longer.

In a preferred embodiment, the anti-NGF antibody is the PG110 antibody or antigen binding fragment thereof, and is administered (e.g., to a human) intravenously at a dose in a range of 0.1 mg/kg to 0.2 mg/kg, preferably 0.15 mg/kg, once every 12 weeks. In another preferred embodiment, an anti-NGF antibody is administered (e.g., to a human) subcutaneously at a dose in a range of 0.2 mg/kg to 0.4 mg/kg, preferably 0.3 mg/kg, once every twelve weeks. In yet other embodiments, PG110 or fragment thereof is administered at a dose in a range of 0.1 mg/kg to 3 mg/kg, or in a range of 0.1 mg/kg to 30 mg/kg, or in a range of 0.1 mg/kg to 20 mg/kg, or in a range of 0.1 mg/kg to 10 mg/kg, or in a range of 1 mg/kg to 30 mg/kg, or in a range of 1 mg/kg to 20 mg/kg or in a range of 1 mg/kg to 10 mg/kg.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. For example, non-limiting examples of dosage unit forms include 0.2 mg (corresponding to a dose of 3 μg/kg in a person of about 70 kg), 2 mg (corresponding to a dose of 30 μg/kg in a person of about 70 kg) and 7 mg (corresponding to a dose of 100 μg/kg in a person of about 70 kg).

### V. METHODS OF USE

The invention provides stable, high concentration compositions with an extended shelf life, which, in one embodiment, are used to inhibit NGF activity in a subject suffering from a disorder in which NGF activity is detrimental. The methods generally comprise administering to the subject a composition of the invention such that NGF activity in the subject is reduced or inhibited. Preferably, the NGF is human NGF and the subject is a human subject. Alternatively, the subject can be a mammal expressing NGF with which an antibody of the invention cross-reacts. Still further the subject can be a mammal into which has been introduced hNGF (e.g., by administration of hNGF or by expression of a hNGF transgene). Moreover, a composition of the invention can be administered to a non-human mammal expressing an NGF with which the antibody cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

A composition of the invention can be administered to a human subject for therapeutic or prophylactic purposes. Accordingly, in another aspect, the invention provides a method of treating, e.g., attenuating or inhibiting, an NGF-related disease or condition in a subject, the method comprising administering to the subject a pharmaceutical composition of the invention. Preferably, the anti-NGF antibody is used to attenuate or alleviate pain, e.g., pain associated with a disease or condition wherein the development or maintenance of the pain is mediated, at least in part, by NGF. Non-limiting examples of NGF-related disease or condition include inflammatory pain, post-surgical pain, post-operative pain (including dental pain), neuropathic pain, peripheral neuropathy, diabetic neuropathy, fracture pain, gout joint pain, post-herpetic neuralgia, cancer pain, osteoarthritis or rheumatoid arthritis pain, sciatica, pains associated with sickle cell crises, headaches (e.g., migraines, tension headache, cluster headache), dysmenorrhea, endometriosis, uterine fibroids, musculoskeletal pain, chronic low back pain, fibromyalgia, sprains, visceral pain, ovarian cysts, prostatitis, chronic pelvic pain syndrome, cystitis, interstitial cystitis, painful bladder syndrome and/or bladder pain syndrome, pain associated with chronic abacterial prostatitis, incisional pain, migraine, trigeminal neuralgia, pain from burns and/or wounds, pain associated with trauma, pain associated with musculoskeletal diseases, ankylosing spondilitis, periarticular pathologies, pain from bone metastases, pain from HIV, erythromelalgia or pain caused by pancreatitis or kidney stones, malignant melanoma, Sjogren's syndrome, asthma, (e.g., uncontrolled asthma with severe airway hyper-responsiveness), intractable cough, demyelinating diseases, chronic alcoholism, stroke, thalamic pain syndrome, pain from toxins, pain from chemotherapy, fibromyalgia, inflammatory bowel disorders, irritable bowel syndrome, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia or allodynia, diabetic neuropathy pain, causalgia, sympathetically maintained pain, deafferentation syndromes, epithelial tissue damage or dysfunction, disturbances of visceral motility at respiratory, genitourinary, gastrointestinal or vascular regions, allergic skin reactions, pruritis, vitiligo, general gastrointestinal disorders, colitis, gastric ulceration, duodenal ulcers, vasomotor or allergic rhinitis, bronchial disorders, dyspepsia, gastroesophageal reflux, pancreatitis, and visceralgia.

Furthermore, NGF has been implicated in the proliferation of cancers such as prostate cancer, thyroid cancer, lung cancer, prolactinoma and melanoma. Accordingly, in another embodiment, the NGF-related disease or condition that can be treated using a pharmaceutical composition of the invention is cancer, preferably prostate cancer, thyroid cancer, lung cancer, prolactinoma or melanoma. Thus, in another embodiment, the invention also provides a method of treating cancer in a subject, preferably prostate cancer, thyroid cancer, lung cancer, prolactinoma or melanoma, comprising administering a pharmaceutical composition of the invention to the subject.

Still further, in another embodiment, the NGF-related disease or condition can be HIV/AIDS. Blockage of NGF using an anti-NGF antibody of the invention may block HIV infected macrophages, thereby treating HIV/AIDS. Accordingly, in another embodiment, the invention also provides a method of treating HIV/AIDS in a subject, comprising administering a pharmaceutical composition of the invention to the subject.

Particularly preferred diseases and conditions for treatment according to the methods of the invention include inflammatory pain (particularly osteoarthritis or rheumatoid arthritis pain), musculoskeletal pain (particularly chronic low back pain), cancer pain, neuropathic pain (particularly diabetic neuropathic pain), pain from bone metastases, interstitial cystitis/painful bladder syndrome, pain associated with chronic abacterial prostatitis, pain from endometriosis and/or uterine fibroids, and post-operative pain.

Pain and/or other symptoms associated with endometriosis and/or uterine fibroids may comprise dysmenorrhoea; chronic non-menstrual, pelvic pain; dyspareunia; dyschexia; menorrhagia; lower abdominal or back pain; infertility and subfertility; dysuria; bloating and pain on micturition; nausea, vomiting and/or diarrohea. Symptoms may also comprise symptoms related to endometriotic lesions or fibroids located outside the peritoneal cavity including for example thoracic endometriosis syndrome manifest as haemoptysis, pneumothorax or haemothorax, and pulmonary leiomyosis manifest as dyspnoea and a pulmonary mass.

In a particularly preferred embodiment, a pharmaceutical composition of the invention is used to treat pain. Preferably, the type of pain treated is selected from the group consisting of osteoarthritis pain, chronic low back pain, diabetic neuropathic pain, cancer pain and endometriosis and/or uterine fibroid pain. Accordingly, in a preferred embodiment, the invention provides a method of treating pain in a subject comprising administering a pharmaceutical composition of the invention such that pain in the subject is treated. Preferably, the pain is selected from the group consisting of osteoarthritis pain, chronic low back pain, diabetic neuropathic pain, cancer pain and endometriosis and/or uterine fibroid pain. Accordingly, in one embodiment, the invention provides a method of treating osteoarthritis pain in a subject comprising administering a pharmaceutical composition of the invention such that osteoarthritis pain in the subject is treated. In another embodiment, the invention provides a method of treating chronic low back pain in a subject comprising administering a pharmaceutical composition of the invention such that chronic low back pain in the subject is treated. In yet another embodiment, the invention provides a method of treating diabetic neuropathic pain in a subject comprising administering a pharmaceutical composition of the invention such that diabetic neuropathic pain in the subject is treated. In yet another embodiment, the invention provides a method of treating cancer pain in a subject comprising administering a pharmaceutical composition of the invention such that cancer pain in the subject is treated. In yet another embodiment, the invention provides a method of treating endometriosis and/or uterine fibroid pain in a subject comprising administering a pharmaceutical composition of the invention such that endometriosis and/or uterine fibroid pain in the subject is treated.

In a preferred embodiment, pharmaceutical composition of the invention comprises an anti-NGF antibody comprising a human IgG4 constant region comprising the amino acid sequence of SEQ ID NO: 10, and alleviates pain in a subject to which the antibody is administered for a long duration. For example, in one embodiment, the antibody alleviates pain for a duration of at least about one week to about twelve weeks (or for at least one week to twelve weeks) after administration of a single dose of the anti-NGF antibody to a subject. In another embodiment, the antibody alleviates pain for a duration of at least about one week (or at least one week) after administration of a single dose of the anti-NGF antibody to a subject. In another embodiment, the antibody alleviates pain for a duration of at least about two weeks (or at least two weeks) after administration of a single dose of the anti-NGF antibody to a subject. In another embodiment, the antibody alleviates pain for a duration of at least about four weeks (or at least four weeks) after administration of a single dose of the anti-NGF antibody to a subject. In another embodiment, the antibody alleviates pain for a duration of at least about eight weeks (or at least eight weeks) after administration of a single dose of the anti-NGF antibody to a subject. In another embodiment, the antibody alleviates pain for a duration of at least about twelve weeks (or at least twelve weeks) after administration of a single dose of the anti-NGF antibody to a subject. In one embodiment, the antibody alleviates pain for a duration of at least about four weeks to about twelve weeks (or for four weeks to twelve weeks) after administration of a single dose of the anti-NGF antibody to a subject. In one embodiment, the antibody alleviates pain for a duration of at least about eight weeks to about twelve weeks (or for eight weeks to twelve weeks) after administration of a single dose of the anti-NGF antibody to a subject.

In another embodiment, the pharmaceutical composition of the invention is administered together with a second pharmaceutical agent or a second treatment regimen. The antibody and the second agent, or the antibody and the second treatment regimen, can be administered or performed simultaneously or, alternatively, the antibody can be administered first, followed by the second pharmaceutical agent or second regimen, or the second pharmaceutical agent or regimen can be administered or performed first, followed by the antibody. Non-limiting examples of suitable second pharmaceutical agents and second treatment regimens are set forth above in the section on pharmaceutical compositions. Particularly referred second pharmaceutical agents for use in combination with an antibody of the invention are opioid analgesics. Other preferred second pharmaceutical agents for use in combination with an antibody of the invention are TrkA inhibitors (e.g., extracellular TrkA inhibitors or intracellular TrkA inhibitors, as described in detail in the section on pharmaceutical compositions) and Protein Kinase C (PKC) inhibitors.

In yet another aspect, the invention provides a method of attenuating or inhibiting a nerve growth factor (NGF)-related disease or condition in a subject such that a rebound effect is avoided in the subject, the method comprising administering to the subject a pharmaceutical composition of the invention comprising an anti-NGF antibody comprising a human IgG4 constant region, wherein the human IgG4 constant region comprises a mutation (preferably a hinge region mutation) and wherein the antibody has a terminal elimination half-life in a cynomolgus monkey of at least 15 days. In another embodiment, the antibody has a terminal elimination half-life in a cynomolgus monkey in a range of about 15 days to about 22 days (or in a range of 15-22 days), or in a range of about 15 days to about 28 days (or in a range of 15-28 days), or in a range of about 21 days to about 28 days (or in a range of 21-28 days). In another embodiment, the antibody has a terminal elimination half-life in a rat of at least 8 days. In yet another embodiment, the antibody has a mean terminal elimination half-life in humans of at least 10-30 days (or at least 10 days, at least 15 days, at least 20 days, at least 25 days, at least 30 days, at least 40 days, or in a range of about 10 days to about 40 days or in a range of 10-40 days or in a range of about 15 to about 30 days or in a range of 15-30 days). Preferred mutations include those described in detail hereinbefore. Preferred antibodies include anti-NGF antibodies of the sequences and/or having the functional properties described in detail hereinbefore.

### VI. ARTICLES OF MANUFACTURE

Also within the scope of the present invention is an autoinjector pen, a prefilled syringe, or a needle-free administration device comprising the liquid pharmaceutical composition of the invention. In one embodiment, the invention features a delivery device comprising a dose of the composition comprising 100 mg/mL of an anti-human NGF antibody, or antigen-binding portion thereof, e.g., an autoinjector pen or prefilled syringe comprises a dose of about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20, mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, or 105 mg of the composition.

Also within the scope of the present invention are kits comprising the pharmaceutical compositions of the invention in liquid or lyophilized form, and optionally include instructions for use in treating an NGF-related disease or condition The kits may include a label indicating the intended use of the contents of the kit. The term label includes any writing, marketing materials or recorded material supplied on or with the kit, or which otherwise accompanies the kit.

For example, the invention also provides a packaged pharmaceutical composition of the invention packaged within a kit or an article of manufacture. The kit or article of manufacture of the invention contains materials useful for the treatment, including prevention, treatment and/or diagnosis of an NGF related disease or condition in a subject. In preferred embodiments, the NGF related disease or condition is inflammatory pain (particularly osteoarthritis or rheumatoid arthritis pain), musculoskeletal pain (particularly chronic low back pain), neuropathic pain (particularly diabetic neuropathic pain), cancer pain (particularly pain from bone metastases), pain associated with endometriosis and/or uterine fibroids, and post-operative pain. The kit or article of manufacture comprises a container and a label or package insert or printed material on or associated with the container which provides information regarding use of the anti-NGF antibody (e.g., PG110), for the treatment of an NGF related disease or condition described herein.

A kit or an article of manufacture refers to a packaged product comprising components with which to administer a pharmaceutical compositions of the invention for treatment of an NGF related disease or condition. The kit preferably comprises a box or container that holds the components of the kit, and can also include a protocol for administering the pharmaceutical composition and/or a “package insert”. The box or container holds components of the invention which are preferably contained within plastic, polyethylene, polypropylene, ethylene, or propylene vessels. For example, suitable containers for the pharmaceutical composition of the invention, include, for example, bottles, vials, syringes, pens, etc.

The term “package insert” is used to refer to instructions customarily included in commercial packages of therapeutic products, that contain information about the indications, usage, dosage, administration, contraindications and/or warnings concerning the use of such therapeutic products. In one embodiment, the package insert of the invention informs a reader, including a subject, e.g., a purchaser, who will be administering the pharmaceutical composition of the invention for treatment, that the pharmaceutical composition of the invention is indicated for treatment of an NGF related disease or condition as described herein. In one embodiment, the package insert describes certain therapeutic benefits of the pharmaceutical composition of the invention, including alleviation of pain. In another embodiment, the package insert can include a description of the dosage of the anti-NGF in the pharmaceutical composition of the invention. In another embodiment, the package insert can include a description of the route and frequency of administration of the pharmaceutical composition of the invention. In another embodiment, the package insert of the invention may also provide information to subjects who will be receiving the pharmaceutical composition of the invention regarding combination uses for both safety and efficacy purposes. For example, in certain embodiments the kit further comprises a second pharmaceutical composition comprising an additional therapeutic packaged with or copromoted with instructions for administration of both agents for the treatment of an NGF-related disease or condition. Particularly preferred diseases and conditions for treatment using the kits of the invention include inflammatory pain (particularly osteoarthritis or rheumatoid arthritis pain), musculoskeletal pain (particularly chronic low back pain), neuropathic pain (particularly diabetic neuropathy), cancer pain and pain from bone metastases, pain associated with endometriosis and/or uterine fibroids, and post-operative pain.

Other embodiments of the present invention are described in the following Examples, which should not be construed as further limiting. The contents of Sequence Listings, figures and all references, patents and published patent applications cited throughout this application are expressly incorporated herein by reference.

### EXAMPLES

The Examples presented below detail experiments performed to examine the effects of solution pH, freeze-thawing, PG110 protein concentration, and various buffers and excipients on the physical and chemical stability of PG110 in order to develop a suitable formulation of PG110.

The following analytical methods were used in experiments performed to assess and monitor the stability of PG110 in solution.

General Methods

PG110 formulations were tested for general quality parameters (e.g., pH), parameters of physical stability (e.g., clarity, color, particle contamination and purity), and parameters of chemical stability, deamidation, oxidation, general chemical stability, and size exclusion chromatography (SEC). Exemplary tests included tests for visible particulate contamination, light obscuration particle count tests for subvisible particles, and tests for purity such as size exclusion HPLC and image capillary ioelectric focusing.

Particulate contamination (e.g., visible particles) was determined by visual inspection. Subvisible particles were monitored by the light blockage method according to United States Pharmacopeia (USP). In addition, the physicochemical stability of formulations was assessed by SEC, which allows for the detection of fragments and aggregates.

To monitor chemical stability, size exclusion high pressure liquid chromatography (SE-HPLC) (for the detection of fragments and hydrolysis in a specimen of a formulation) and icIEF (image capillary isoelectric focusing) were performed.

icIEF Methods

icIEF analyses were performed using the iCE280 imaging cIEF system with a PrinCE autosampler (Covergent Biosciences). The Table 1 below lists the reagents and materials used for the icIEF analyses.

TABLE 1

| Reagent | Manufacturer | Product ID |
|---|---|---|
| 1% Methyl cellulose solution | Convergent Bioscience | 101876 |
| 0.5% Methyl cellulose solution | Convergent Bioscience | 102505 |
| Pharmalyte 3-10 | GE healthcare | 17-0456-01 |
| 0.08M H3PO4 in 0.1% Methyl | Convergent Bioscience | 102506 |
| cellulose solution (anolyte) | | |
| 0.1M NaOH in 0.1% Methyl | Convergent Bioscience | 102506 |
| cellulose solution (catholyte) | | |
| pI 5.12 marker | Convergent Bioscience | 102224 |
| pI 9.22 marker | Convergent Bioscience | 102231 |

The iCE280 instrument was operated according to manufacturer instructions. Respective vials were filled with fresh anolyte and catholyte solutions, the waste vial was filled with MilliQ HPLC water, and the UV lamp was turned on.

pI markers were prepared by diluting both pI 5.12 and pI 9.22 markers 10-fold with MilliQ HPLC water, and mixing well.

PG110 samples for analysis were prepared by diluting PG110 test samples to 1 mg/mL with MilliQ HPLC water, combining the diluted antibody solution with the components in the table below, and vortexing briefly. Samples were subsequently transferred to glass inserts seated in autosampler tubes and degassed for 5 minutes before placement into a PrinCE autosampler.

TABLE 2

| Component | Volume (μL) |
|---|---|
| 1% Methyl cellulose | 70 |
| Pharmalyte 3-10 | 8 |
| Diluted pI 5.12 marker | 8 |
| Diluted pI 9.22 marker | 8 |
| 1 mg/mL sample | 50 |
| Water | 56 |

Size Exclusion HPLC Methods

Size exclusion HPLC was used to determine the purity of PG110 solutions. The assay was performed as outlined below.

A TSK gel guard (cat. no. 08543, 6.0 mm×4.0 cm, 7 μm), was combined with a TSK gel G3000SW (cat. no. 08541, 7.8 mm×30 cm, 5 μm) and run with an upper column pressure limit of 70 bar. The mobile phase consisted of 100 mM Na₂HPO₄/200 mM Na₂SO₄, pH 7.0. This buffer was created by dissolving 49.68 g anhydrous disodium hydrogen phosphate and 99.44 g anhydrous sodium sulfate in approximately 3300 mL Milli-Q water, adjusting the pH to 7.0 using 1 M phosphoric acid, increasing the buffer volume to 3500 mL with Milli-Q water and filtering the solution through a membrane filter.

Experimental parameters were as follows:
0.3 ml/min flow rate
20 μL injection volume (equivalent to 20 μg sample)
room temperature column
2 to 8° C. autosampler temperature
50 minute run time
isocratic gradient

Detection was performed using a diode array detector using a 214 nm wavelength (>0.1 min peak width and 8 nm band width) and a 360 nm reference wavelength (100 nm band width).

Test samples were injected in duplicate. Purity was determined by comparing the area of PG110 antibody peak to the total area of all 214 nm absorbing components in the sample, excluding buffer-related peaks. High molecular weight aggregates and antibody fragments were resolved from intact PG110 using this method.

Light Obscuration

Light obscuration assays were performed to measure the insoluble particulate content of antibody solutions. Light obscuration measurement equipment (particle counter, model syringe, Klotz (Bad Liebenzell, Germany, series S20037) was equipped with laminar air hood (Thermo Electron Corp., Asheville, N.C., model no. ULT2586-9-A40) to minimize foreign particle contamination during measurements. Light obscuration analysis was performed as follows. A 3.5 mL sample was placed in a 5 mL round-bottom tube under laminar air flow conditions. Measurements were performed according to manufacturer's specifications in n=3 mode (0.8 mL per single measurement), after an initial 0.8 mL rinse.

Differential Scanning calorimetry (DSC)

Prior to DSC analysis, proteins are dialyzed into a suitable buffer system using Slide-A-Lyzer Cassettes. This buffer system (10 mM phosphate, 10 mM citrate) is also used as a reference/blank for the DSC measurement. The antibody is analyzed at 1-2 mg/mL. An automated VP-DSC with Capillary Cell (Microcal) DSC instrument is used. Unfolding of the molecules is studied applying a 1° C./minute scan rate over a 25° C.-95° C. temperature range. Other measurement parameters are: Fitting period: 16 sec, pre-scan wait: 10 min, feedback mode: none.

Visual Inspection

Visual Inspection of the protein samples were performed by carefully inspecting the protein solution in the sample container with the unaided eye. Typically, the samples are inspected against a white and a dark/black background to more readily identify visible particulate matter, haziness, opalescence, or protein precipitate and visible particles and agglomerates. Sample containers amenable for visual inspection can vary, and may include containers such as translucent and clear Falcon tubes, glass vials, low-volume vials/tubes, and slide-a-lyzer cassettes.

### Example 1

### Impact of Solution pH on The Stability of PG110 Formulations During Repeated Freeze/Thaw Studies (−80° C./30° C.).

The freeze thaw behavior of the ABT110 antibody at a protein concentration of 1 mg/mL in 10 mM citrate/10 mM phosphate buffer was evaluated by cycling the protein solution up to 4 times between the frozen state and the liquid state at pH 4, pH 5, pH 6, pH 7, and pH 8. Freezing was performed by means of a temperature controlled −80° C. freezer, and thawing was performed by means of a 30° C. temperature controlled water bath. Samples were pulled after each freeze/thaw (F/T) cycle and analyzed by SEC. About 20 mL of each PG110 solution was placed in 30 mL PETG repositories for this experiment. Table 3 provides an overview on testing intervals for SEC and the number of freeze/thaw cycles performed. Table 4 shows the effect of freeze/thaw processing on the amount of monomer of PG110 remaining and the amount of fragments and aggregates formed in the samples formulated at these pH levels.

TABLE 3
Testing Intervals: Number Of Freeze (−80° C.)
And Thaw (30° C. Water Bath) Cycles Tested

| | Testing Intervals: Number of | |
|---|---|---|
| | Freeze/Thaw Cycles and | |
| | Sample Requirements for Testing | |
| Storage Temperature For Stress Test | T₀ | 4 |
| −80° C./30° C. Cycling Study | 1 | 1 |

TABLE 4
Physical Stability Of PG110 During Repeated
Freeze/Thaw Cycling As Determined Via SEC

| | | | | | |
|---|---|---|---|---|---|
| f/t cycles | pH 4 | pH 5 | pH 6 | pH 7 | pH 8 |

Monomer

| | | | | | |
|---|---|---|---|---|---|
| T0 | 98.53 | 98.46 | 98.39 | 98.11 | 98.00 |
| T4 | 96.74 | 96.46 | 97.81 | 97.91 | 97.55 |

Aggregates

| | | | | | |
|---|---|---|---|---|---|
| T0 | 1.31 | 1.41 | 1.47 | 1.74 | 1.85 |
| T4 | 3.00 | 3.34 | 2.00 | 1.94 | 2.24 |

Fragments

| | | | | | |
|---|---|---|---|---|---|
| T0 | 0.15 | 0.11 | 0.13 | 0.14 | 0.14 |
| T4 | 0.24 | 0.18 | 0.18 | 0.13 | 0.19 |

The results show that the amount of PG110 monomer slightly decreased during repeated freeze/thaw (F/T) processing, however, only to a small extent and more than 95% of intact monomer remained stable in solution.

Light obscuration experiments were conducted to determine the number of subvisible particles formed during each freeze/thaw step. Table 5 provides an overview on testing intervals for light obscuration and the number of freeze/thaw cycles performed. Tables 6 and 7 show the effect of freeze/thaw processing on the number of particles of size greater than equal to 1 micrometer/mL and greater than equal to 10 micrometer, respectively.

TABLE 5
Testing Intervals: Number Of Freeze (−80° C.)
And Thaw (30° C. Water Bath) Cycles Tested

| | Testing Intervals: Number of | |
|---|---|---|
| | Freeze/Thaw Cycles and | |
| | Sample Requirements for Testing | |
| Storage Temperature For Stress Test | T₀ | 3 |
| −80° C./30° C. Cycling Study | 2 | 2 |

TABLE 6
Physical Stability Of PG110 During Repeated Freeze/Thaw Cycling As Determined via
sub visible particle measurements by light obscuration technique. Particles of size
greater than equal to 1 micron/mL (data represent the average of two measurements)

| | | Deviation | | Deviation | | Deviation | | Deviation |
|---|---|---|---|---|---|---|---|---|
| | 0 F/T | from average | 1 F/T | from average | 2 F/T | from average | 3 F/T | from average |
| Water/control | 15 | 3.75 | 30.41 | 7.5 | 19.37 | 4.79 | 29.37 | 9.79 |
| pH 4 | 3605 | 140 | 8576 | 7716 | 10524 | 1432 | 45162 | 2117 |
| pH 5 | 2150 | 1595 | 14793 | 1976 | 26302 | 8870 | 74402 | 9673 |
| pH 6 | 207 | 4.58 | 53577 | 6670 | 30601 | 4386 | 75999 | 10809 |
| pH 7 | 140 | 19 | 41932 | 4279 | 32737 | 50 | 54267 | 2828 |
| pH 8 | 137 | 1.25 | 18862 | 2643 | 21725 | 1407 | 48981 | 623 |

TABLE 7
Physical Stability Of PG110 During Repeated Freeze/Thaw Cycling As Determined
Via sub visible particle measurements by light obscuration technique.

| | | Deviation | | Deviation | | Deviation | | Deviation |
|---|---|---|---|---|---|---|---|---|
| | | from | | from | | from | | from |
| | 0 F/T | average | 1 F/T | average | 2 F/T | average e | 3 F/T | average |
| Water/control | 0 | 0 | 2.08 | 2.08 | 1.25 | 0 | 0.79 | 0.79 |
| pH 4 | 55.62 | 16 | 121 | 105 | 1375 | 147 | 3142 | 2789 |
| pH 5 | 41 | 31 | 744 | 390 | 9293 | 5575 | 20507 | 14028 |
| pH 6 | 5.62 | 0.62 | 993 | 253 | 3823 | 3.54 | 8039 | 1785 |
| pH 7 | 4.58 | 0.41 | 494 | 49 | 3932 | 21 | 6517 | 1167 |
| pH 8 | 4.79 | 1.45 | 301 | 244 | 4019 | 216 | 4063 | 735 |

Particles of size greater than equal to 10 micron/mL (data represent the average of two measurements).

### Example 2

### Impact of Solution pH On Physico-Chemical Stability of PG110 Formulations During Accelerated Storage

Important factors influencing protein stability during accelerated/long-term storage of protein liquid and lyophilized formulations are the pH of the formulations and the storage temperature. To assess the impact of these factors, the protein was exposed to short-term storage at elevated temperatures during preformulation and formulation project stages in order to quickly gain insight in the formulation feasibility for long-term storage at lower temperatures (e.g., 2-8° C.).

Storage stability of the PG110 antibody in solution (2 mg/mL, 10 mM citrate/10 mM phosphate buffer) was evaluated at various temperatures for prolonged periods of time at controlled temperature conditions. After defined storage periods, samples were pulled and the impact of storage time and storage temperature on PG110 stability was evaluated.

For this pH screening study, PG110 was formulated at pH 3, pH 4, pH 5, pH 6, pH 7, and pH 8 at 2 mg/mL in 10 mM phosphate, 10 mM citrate.

Samples were filled into sterile vials (approx. 500 μL each) and stored under controlled conditions (in temperature chambers and in the absence of light) at 40° C. and 50° C. At predefined points of time, samples of prepared solutions were pulled for analysis according to the sample pull scheme provided in Table 8. Numbers refer to number of vials that were stored/pulled. The resulting data is provided in Tables 9 and table 10.

TABLE 8
Sample Pull Scheme

| | T0 | 7 days | 6 m | 12 m |
|---|---|---|---|---|
| 5° C. | | | 1 | 1 |
| 25° C. | | | 1 | |
| 40° C. | 1 | 1 | | |
| 50° C. | | 1 | | |

TABLE 9
Monomer, Aggregate and Fragment Content Of PG110 Samples
Formulated At Various pH After Long-Term Storage (SEC
Data) when stored at 50° C.
PG110 stored at 50° C.

| | | | | | | |
|---|---|---|---|---|---|---|
| Time | pH 3 | pH 4 | pH 5 | pH 6 | pH 7 | pH 8 |

Monomer

| | | | | | | |
|---|---|---|---|---|---|---|
| 0 | 0 | 96.85 | 97 | 97.15 | 96.93 | 96.68 |
| 7 days | 0 | 9.65 | 85.54 | 94.84 | 93.96 | 92.2 |

Aggregate

| | | | | | | |
|---|---|---|---|---|---|---|
| 0 | 100.00 | 1.75 | 1.75 | 1.62 | 1.95 | 2.12 |
| 7 days | 100.00 | 86.22 | 12.41 | 3.06 | 4.00 | 5.19 |

Fragment

| | | | | | | |
|---|---|---|---|---|---|---|
| 0 | 0.00 | 1.39 | 1.23 | 1.21 | 1.10 | 1.18 |
| 7 days | 0.00 | 4.12 | 2.03 | 2.08 | 2.03 | 2.60 |

TABLE 10
Monomer, Aggregate and Fragment Content Of PG110 Samples
Formulated At Various pH After Long-Term Storage (SEC
Data) when stored at various temperatures.
PG110 stored at Various temperatures

| | | | | | | |
|---|---|---|---|---|---|---|
| Time | pH 3 | pH 4 | pH 5 | pH 6 | pH 7 | pH 8 |

Monomer

| | | | | | | |
|---|---|---|---|---|---|---|
| 0 | 0 | 96.85 | 97 | 97.15 | 96.93 | 96.68 |
| 7 days, 40 C. | 0 | 81.64 | 96.64 | 96.84 | 96.1 | 95.49 |
| 6 months, 25 C. | 0 | 92.89 | 94.81 | 94.32 | 95.14 | 89.12 |
| 6 months, 5 C. | 0 | 97.87 | 97.86 | 97.78 | 97.33 | 97.10 |
| 12 months, 5 C. | 0 | 97.50 | 97.37 | 97.53 | 97.11 | 96.42 |

Aggregate

| | | | | | | |
|---|---|---|---|---|---|---|
| 0 | 100 | 1.75 | 1.75 | 1.62 | 1.95 | 2.12 |
| 7 days, 40 C. | 100 | 15.32 | 1.78 | 1.65 | 2.56 | 3.08 |
| 6 months, 25 C. | 100 | 2.55 | 2.38 | 3.02 | 2.71 | 2.81 |
| 6 months, 5 C. | 100 | 1.38 | 1.56 | 1.64 | 1.95 | 2.22 |
| 12 months, 5 C. | 100 | 1.40 | 1.62 | 1.57 | 2.04 | 2.67 |

Fragment

| | | | | | | |
|---|---|---|---|---|---|---|
| 0 | 0 | 1.39 | 1.23 | 1.21 | 1.1 | 1.18 |
| 7 days, 40 C. | 0 | 3.03 | 1.57 | 1.49 | 1.33 | 1.42 |
| 6 months, 25 C. | 0 | 4.55 | 2.79 | 2.64 | 2.14 | 8.05 |
| 6 months, 5 C. | 0 | 0.73 | 0.57 | 0.56 | 0.71 | 0.66 |
| 12 months, 5 C. | 0 | 1.08 | 1.00 | 0.88 | 0.83 | 0.90 |

Data for the image capillary iso-electric focusing for the above mentioned samples of accelerated stability was also evaluated. icIEF provides information on the chemical stability of the molecule. Tables 11 and 12 show the sample pull scheme and the data, respectively.

TABLE 11
Sample Pull Scheme

| | T0 | 21 days | 4 months | 12 months |
|---|---|---|---|---|
| 40° C. | 1 | 1 | 1 | |
| 25° C. | | | 1 | |
| 5° C. | | | 1 | 1 |

TABLE 12
Content Of the main, acidic and the basic species of PG110
Samples Formulated At Various pH After Long-Term Storage
(iCIEF) when stored at various temperatures. The results
also show the corresponding pI of the molecules.

| | | | | |
|---|---|---|---|---|
| Sample | Acidic | Main | Basic | pI |

T0

| | | | | |
|---|---|---|---|---|
| pH 4.0 | 33.27 | 55.38 | 11.35 | 7.01 |
| pH 5.0 | 33.98 | 56.63 | 9.39 | 6.95 |
| pH 6.0 | 32.57 | 57.13 | 10.3 | 7.01 |
| pH 7.0 | 31.71 | 58.72 | 9.57 | 7.1 |
| pH 8.0 | 31.98 | 59.03 | 8.99 | 7.12 |

40° C. 21 day

| | | | | |
|---|---|---|---|---|
| pH 4.0 | 24.87 | 23.81 | 51.33 | 6.94 |
| pH 5.0 | 46.38 | 44.1 | 9.53 | 6.94 |
| pH 6.0 | 48.31 | 43.42 | 8.27 | 6.94 |
| pH 7.0 | 56.99 | 37.06 | 5.95 | 6.94 |
| pH 8.0 | 74.27 | 19.43 | 6.3 | 6.93 |

5° C. 4 months

| | | | | |
|---|---|---|---|---|
| pH 4.0 | 35.50 | 52.96 | 11.54 | 7.02 |
| pH 5.0 | 33.43 | 56.73 | 9.84 | 7.01 |
| pH 6.0 | 33.62 | 57.09 | 9.30 | 7.01 |
| pH 7.0 | 34.56 | 56.49 | 8.95 | 7.01 |
| pH 8.0 | 37.02 | 54.31 | 8.68 | 7.01 |

25° C. 4 months

| | | | | |
|---|---|---|---|---|
| pH 4.0 | 52.31 | 29.10 | 18.59 | 7.01 |
| pH 5.0 | 44.39 | 45.58 | 10.03 | 7.02 |
| pH 6.0 | 43.76 | 47.81 | 8.43 | 7.01 |
| pH 7.0 | 53.44 | 40.08 | 6.48 | 7.01 |
| pH 8.0 | 70.80 | 26.00 | 3.20 | 6.99 |

40° C. 4 months

| | | | | |
|---|---|---|---|---|
| pH 4.0 | 19.02 | 9.62 | 71.36 | 6.99 |
| pH 5.0 | 74.96 | 7.37 | 17.68 | 7.01 |
| pH 6.0 | 85.28 | 8.94 | 5.78 | 7.01 |
| pH 7.0 | 95.74 | 4.26 | 0.00 | 6.98 |
| pH 8.0 | 99.07 | 0.36 | 0.57 | 6.98 |

5° C. 12 Months

| | | | | |
|---|---|---|---|---|
| pH 4.0 | 37.21 | 52.58 | 10.21 | 7.17 |
| pH 5.0 | 35.45 | 55.20 | 9.35 | 7.16 |
| pH 6.0 | 34.95 | 55.48 | 9.57 | 7.14 |
| pH 7.0 | 36.76 | 54.55 | 8.69 | 7.14 |
| pH 8.0 | 42.54 | 49.29 | 8.17 | 7.12 |

These data demonstrate that a solution pH range of about pH 5-7 maintains PG110 stability best at increased temperatures. After 1 week of storage at 40° C. and 50° C., monomer levels were highest in samples formulated at pH 6.

PG110 at 2 mg/mL outside of a pH 5-7 range clearly induced stability loss, mirrored by increased levels of aggregates and fragments. Fragment levels revealed a minimum of degradation in samples formulated at pH of about 6. The icIEF data also shows that a pH of about 6 was best to maintain stability of PG110. These data suggest that a pH of about 5.5-6.5 maintains PG110 protein stability best at the specific stress conditions applied in this experiment.

### Example 3

### Impact of formulations on The Stability of PG110 Formulations During Repeated Freeze/Thaw Studies (−80° C./30° C.) at 30 mg/mL Conditions

The freeze/thaw (F/T) behavior of the ABT110 antibody at a protein concentration of 30 mg/mL in different formulations was evaluated by cycling drug substance up to 3 times between the frozen state and the liquid state at pH 5.5. The formulations that were evaluated are:
(1) 10 mM acetate+125 mM sodium chloride pH 5.5
(2) 15 mM Histidine pH 5.5
(3) 15 mM histidine and 0.01% Tween 80 pH 5.5

Freezing was performed by means of a temperature controlled −80° C. freezer, and thawing was performed by means of a 30° C. temperature controlled water bath. Samples were pulled after each freeze/thaw cycle and analyzed by SEC and visual inspection. About 1 mL of PG110 solution were placed in repositories for this experiment. Table 13 provides an overview on testing intervals for SEC and the number of freeze/thaw cycles performed. Table 14 shows the effect of freeze/thaw processing on the amount of monomer of PG110 remaining and the amount of fragments and aggregates formed in the samples formulated at these pH levels.

TABLE 13
Testing Intervals: Number Of Freeze (−80° C.)
And Thaw (30° C. Water Bath) Cycles Tested

| | Testing Intervals: Number of | |
|---|---|---|
| | Freeze/Thaw Cycles and | |
| | Sample Requirements for Testing | |
| Storage Temperature For Stress Test | T₀ | 3 |
| −80° C./30° C. Cycling Study | 1 | 1 |

TABLE 14
Physical Stability Of PG110 During Repeated
Freeze/Thaw Cycling As Determined Via SEC

| | | | |
|---|---|---|---|
| T0 | 1 F/T | 2 F/T | 3 F/T |

Acetate + NaCl pH 5.5

| | | | | |
|---|---|---|---|---|
| Monomer | 98.76 | 98.76 | 98.76 | 98.83 |
| Aggregate | 1.23 | 1.23 | 1.23 | 1.16 |
| Fragment | 0 | 0 | 0 | 0 |

15 mM Histidine, pH 5.5

| | | | | |
|---|---|---|---|---|
| Monomer | 98.82 | 98.82 | 98.83 | 98.85 |
| Aggregate | 1.17 | 1.17 | 1.16 | 1.14 |
| Fragment | 0 | 0 | 0 | 0 |

15 mM Histidine, 0.01% Tween 80, pH 5.5

| | | | | |
|---|---|---|---|---|
| Monomer | 98.85 | 98.85 | 98.89 | 98.86 |
| Aggregate | 1.14 | 1.14 | 1.1 | 1.13 |
| Fragment | 0 | 0 | 0 | 0 |

The visual inspection of the various formulations showed that the histidine and tween 80 (polysorbate 80) containing formulations had minimal particle formation even after 3 F/T cycles, indicating that both histidine and Tween 80 are very suitable excipients for maintaining PG110 stability. The other two formulations showed much higher number of visible particles (20-30 visible particles per container).

### Example 4

### Impact of Formulation Parameters on the Stability of PG110 Formulations During Microcalorimetry Studies (Intrinsic Stability) at 1 mg/mL Conditions

The thermodynamic stability (intrinsic stability) of the ABT110 antibody at a protein concentration of 1 mg/mL in different formulations was evaluated by using microcalorimetry. Heating was performed at a scan rate of 1° C./minute. The results are summarized in Table 15.

TABLE 15
The melting transition temperatures under
different formulation conditions.

| | Tm1 | Tm2 | Tm3 |
|---|---|---|---|
| 15 mM Histidine, pH 6 | 58.59 | 67.25 | 75.02 |
| 15 mM Phosphate, pH 6 | 68.3 | 74.68 | 77.22 |
| 15 mM Succinate, pH 6 | 68.4 | 74.62 | 77.09 |
| 10 mM Acetate + 125 mM NaCl, pH 5.5 | 65.5 | 72.99 | 76.21 |
| Water, pH 6 | 69.82 | 75.58 | 77.81 |
| 10 Mm Citrate + 10 mM Phosphate + | 67.8 | 73.96 | 76.69 |
| 0.01% Tween 80, pH 6 | | | |
| 10 Mm Citrate + 10 mM Phosphate + | 68.5 | 74.52 | 77.2 |
| 40 mg/mL Mannitol, pH 6 | | | |
| 10 Mm Citrate + 10 mM Phosphate + | 68.9 | 74.9 | 77.34 |
| 40 mg/mL Sorbitol, pH 6 | | | |
| 10 Mm Citrate + 10 mM Phosphate + | 68.75 | 74.73 | 77.41 |
| 40 mg/mL Sucrose, pH 6 | | | |
| 10 Mm Citrate + 10 mM Phosphate + | 68.9 | 74.91 | 77.55 |
| 80 mg/mL Trehalose, pH 6 | | | |
| 10 Mm Citrate + 10 mM Phosphate, pH 4 | 53.68 | 62.02 | 69.68 |
| 10 Mm Citrate + 10 mM Phosphate, pH 6 | 67.92 | 74.41 | 76.78 |
| 10 Mm Citrate + 10 mM Phosphate, pH 8 | 70.56 | 75.58 | 77.42 |

These data show that the intrinsic stability of PG110 is impacted by formulation parameters, e.g., formulation pH and excipients.

### Example 5

### Impact of Concentration On The Stability of PG110 Formulation During Repeated Freeze/Thaw Studies (−80° C./30° C.) at 100 mg/mL Conditions

The freeze/thaw (F/T) behavior of the ABT110 antibody at a protein concentration of 100 mg/mL was evaluated by cycling the protein solution up to 4 times between the frozen state and the liquid state at pH 6. Previous data indicates that histidine is a suitable buffer/excipient for stabilization of PG110 and, thus, the stabilizing impact of histidine on PG110 protein stability was tested at 100 mg/mL protein concentration.

Freezing was performed by means of a temperature controlled −80° C. freezer, and thawing was performed by means of a 30° C. temperature controlled water bath. Samples were pulled after each freeze thaw cycle and analyzed by SEC and visual inspection. Table 16 provides an overview on testing intervals for SEC and the number of freeze/thaw cycles performed. Table 17 shows the effect of freeze/thaw processing on the amount of monomer of PG110 remaining and the amount of fragments and aggregates formed in the samples formulated at these pH levels.

TABLE 16
Testing Intervals: Number Of Freeze (−80° C.)
And Thaw (30° C. Water Bath) Cycles Tested

| | Testing Intervals: Number of | |
|---|---|---|
| | Freeze/Thaw Cycles and | |
| | Sample Requirements for Testing | |
| Storage Temperature For Stress Test | T₀ | 1 |
| −80° C./30° C. Cycling Study | T2 | 2 |
| −80° C./30° C. Cycling Study | T4 | 2 |

TABLE 17
Physical Stability Of PG110 formulated at high protein
concentration (100 mg/mL) in pH 6, 15 mM histidine, during
repeated freeze/thaw cycling As determined Via SEC.

| SEC data | Sample | T0 | 2 F/T | 4 F/T |
|---|---|---|---|---|
| Monomer | sample 1 | 98.0 | 97.9 | 97.9 |
| | sample 2 | — | 97.9 | 97.9 |
| Aggregate | sample 1 | 1.9 | 1.9 | 1.9 |
| | sample 2 | — | 1.9 | 1.9 |
| Fragment | sample 1 | 0.1 | 0.2 | 0.2 |
| | sample 2 | — | 0.2 | 0.2 |

The data show that at 100 mg/mL, PG110 formulations did not undergo physical instability during repeated f/t processing, since monomer, aggregate and fragment levels virtually remained unchanged throughout the f/t experiment, indicating that histidine is a very suitable excipient for maintaining PG110 stability during f/t processing.

### Example 6

### Impact of Buffers and Excipients on the Turbidity and Morphology of Particles within PG110 Formulations after Dialysis as Determined by Visual Inspection

Earlier experience with PG110 has shown that the protein is prone to physical instability, as reflected by severe visible particle formation and precipitation phenomena when stored in a solution of 10 mM acetate, 125 mM NaCl at pH 5.5. This experiment was designed to verify if the visible particle formation is inherent to the protein itself or whether a formulation can be identified that maintains physical stability and reduces the particle formation susceptibility.

Since the aforementioned particles can be observed with the naked eye, a careful visual inspection of PG110 solutions formulated with different excipients is a very informative way to determine what formulation conditions can accelerate or prevent particle formation.

To carry this out, solutions of PG110 with the excipients listed in Table 18 and at a concentration of 1 mg/ml were prepared by dialysis.

TABLE 18
Buffers and excipients evaluated for their effect on PG110
visible particle formation in solution (universal buffer
or UB6 is 10 mM phosphate, 10 mM citrate pH 6).

15 mM sodium phosphate
15 mM sodium citrate
15 mM sodium succinate
15 mM arginine
15 mM histidine
Self-buffering formulation
10 mM universal buffer and 40 mg/mL mannitol
10 mM universal buffer and 40 mg/mL sorbitol
10 mM universal buffer and 80 mg/mL sucrose
10 mM universal buffer and 80 mg/mL trehalose
10 mM universal buffer and 0.01% (m/m) polysorbate 80
10 mM acetate, 125 mM NaCl

PG110 solution greater than 1 mg/ml was inserted into slide-a-lyzer cassettes with 10,000 MWCO and dialyzed against 1 L of the target buffer/excipient medium for 1 hour. Afterwards, the dialysis medium was replaced by fresh medium and the dialysis was continued overnight. Following dialysis, the concentration of the solutions was measured by UV280. If the concentration was too high, solutions were diluted with the corresponding buffer to the target concentration. If the concentration was too low, the solution was concentrated with Amicon Ultra centrifuge tubes to the target concentration. Next, the pH of the solutions was checked. If the pH was not within ±0.1 of 6, the pH was adjusted to that target with 0.1 M NaOH or 0.1 M HCl. The condition of pH 6 was chosen based upon prior experiments which determined that it was near the optimal pH for chemical and physical stability. Afterwards, the solutions were passed through 0.20 μm filters into clear PETG containers. Distilled water was also passed through the same filters into PETG containers to serve as a control.

Following this procedure, PG110 solutions in the PETG vials were visually inspected for particles. The bottles were held against a soft fluorescent light as well as against a black background. The bottles were also gently shaken to cause the particles to flow, thus rendering visual inspection easier. The bottles were then stored at 4° C. overnight. The next day, the bottles were removed from storage and inspected as above.

Inspection immediately after filtration revealed no visible particles in all samples. However, after storage overnight at 4° C., visual inspection revealed particle formation in many of the buffers/excipients. The findings are summarized in Table 19.

TABLE 19
Visual inspection findings of solutions of PG110 in
the listed buffers/excipients. The solutions were inspected
after filtration and storage overnight at 4° C.
UB6 is 10 mM citrate, 10 mM phosphate pH 6.

| Buffer/Excipient | Visual Observation |
|---|---|
| water | no particles |
| 15 mM phosphate | dust-like fibers |
| 15 mM citrate | dust-like fibers |
| 15 mM succinate | lots of dust-like fibers |
| 15 mM histidine | trace amounts of dust-like fibers |
| 15 mM arginine | trace amounts of dust-like fibers |
| self buffering/just water | very small trace amounts of dust-like |
| | fibers |
| UB6 + 40 mg/ml sorbitol | dust-like fibers |
| UB6 + 40 mg/ml mannitol | trace amounts of dust-like fibers |
| UB6 + 80 mg/ml sucrose | dust-like fibers |
| UB6 + 80 mg/ml trehalose | dust-like fibers |
| UB6 + 0.01% Tween80 | Clear. No particles |
| 10 mM acetate, 125 mM NaCl | lots of dust-like fibers |

The data indicate that Tween-80 prevents the formation of visible particles, justifying its use. Of the given excipients that have buffering capacity at pH 6 (citrate, phosphate, succinate, histidine), the data indicate that histidine is best for preventing visible particle formation.

### Example 7

### Impact of Buffers and Formulation Excipients on the Stability of PG110 Formulations during Repeated Freeze/Thaw Cycles (−80° C./30° C.)

This example describes data of experiments conducted to evaluate the stabilization potential of various buffers and excipients in formulations of PG110 solutions at 2 mg/mL and pH of 6 upon repeated freeze (−80° C. temperature controlled freezer) and thaw (30° C. temperature controlled circulating water bath) processing. (The condition of pH 6 was chosen based upon prior experiments which determined that it was near the optimal pH for chemical and physical stability). Buffers and excipients tested are listed in Table 20.

TABLE 20
Buffers and excipients evaluated for their effect on PG110
DS degradation when exposed to freeze-thawing (universal
buffer or UB6 is 10 mM phosphate, 10 mM citrate pH 6).

15 mM sodium phosphate
15 mM sodium citrate
15 mM sodium succinate
15 mM arginine
15 mM histidine
Low-ionic formulation (i.e. formulating in water)
10 mM universal buffer and 40 mg/mL mannitol
10 mM universal buffer and 40 mg/mL sorbitol
10 mM universal buffer and 80 mg/mL sucrose
10 mM universal buffer and 80 mg/mL trehalose
10 mM universal buffer and 0.01% (m/m) polysorbate 80
10 mM acetate, 125 mM NaCl

Samples were pulled at T0, T1 (after one freeze/thaw step), T2, and T3. One freeze-thaw processing step encompassed sample storage at −80° C. for at least 4 hours and subsequent thawing of the sample in a 30° C. circulating water bath. To analyze freeze-thaw samples, 5 mL round-bottom tubes were filled with 3.5 mL of antibody formulation (using a 5 mL pipette tip that has been rinsed with 0.2 μm filtered WFI) and subjected to light obscuration measurement. Furthermore, 0.1 mL of each sample was pulled for SEC analysis, and 0.2 mL of sample were pulled and stored at −80° C. (reserve sample for optional additional analytical characterization).

TABLE 21
Sample Pull Scheme for Freeze-Thaw Experiments

| | T0 | T1 | T2 | T3 | T4 |
|---|---|---|---|---|---|
| −80° C./30° C. | Vial 1 | Vial 1 | Vial 1 | Vial 1 | Vial 1 |
| | Vial 2 | Vial 2 | Vial 2 | Vial 2 | Vial 2 |

* Vial denotes 30 mL PETG repository filled with sample solution

The effect of buffers and excipients on the formation of subvisible particles of size ≧1 μm and ≧10 μm during freeze thaw processing of PG110 is shown in Tables 22 and 23, respectively. SEC data is given in Tables 24, 25, and 26.

In some formulations, such as those with phosphate, citrate, sorbitol, mannitol, and sucrose, the number of particles ≧1 μm/mL increased after the first freeze-thaw cycle only to decrease after the second cycle. In other formulations, such as those containing histidine, arginine, or simply water, the number of particles ≧1 μm/mL increased after every freeze-thaw cycle. Particles ≧10 μm/mL increased after every freeze-thaw cycle for formulations with phosphate, citrate, succinate, histidine, arginine, and simply water. For formulations with sorbitol, mannitol, or sucrose particles ≧10 μm/mL increased after the first freeze-thaw cycle, but decreased with subsequent cycles. After one freeze-thaw cycle, formulations with sorbitol or mannitol had the greatest number of particles ≧1 μm/mL (at least >˜200,000) and also ≧10 μm/mL (˜25000 average). However, after the third freeze-thaw cycle, all formulations revealed particles ≧1 μm/mL of less than 100,000 per mL.

Polysorbate 80 was found to have a positive effect with regard to maintaining PG110 stability, as it prevented the formation of subvisible particles during the freeze thaw processing of PG110. This is attributed to the polysorbate 80's ability to prevent the denaturation of the antibody at the ice-water interface. Sugars/sugar alcohols including mannitol, sorbitol, and sucrose were found induce subvisible particle formation after early freeze-thaw cycles. These observations are supported by the SEC data which show a noticeable loss in % monomer and a corresponding increase in % aggregate for formulations with mannitol and sorbitol. (For all other excipients, SEC data does not differentiate in terms of stability.)

TABLE 22
Number of particles ≧1 μm/mL measured after the listed freeze-thaw cycles.

| | F/T 0 | | F/T 1 | | F/T 2 | | F/T 3 | |
|---|---|---|---|---|---|---|---|---|
| | # >= 1 um | | # >= 1 um | | # >= 1 um | | # >= 1 um | |
| Buffer | ave | sd | ave | sd | ave | sd | ave | sd |
| phosphate | 536.67 | 94.58 | 17274.58 | 6372.51 | 9415.42 | 2353.78 | 9014.58 | 512.36 |
| citrate | 723.75 | 174.13 | 40620.83 | 795.79 | 27191.25 | 10421.87 | 10470.21 | 7553.08 |
| succinate | 1147.29 | 571.58 | 20720.21 | 2263.33 | 19527.29 | 8585.45 | 9504.17 | 1297.25 |
| histidine | 250.21 | 42.43 | 1647.08 | 617.25 | 8963.33 | 4741.45 | 21077.50 | 12656.92 |
| arginine | 690.63 | 557.44 | 2407.29 | 122.57 | 5858.54 | 2044.13 | 8314.17 | 6856.28 |
| self/water | 410.21 | 23.57 | 1622.50 | 811.11 | 8722.50 | 4459.19 | 21048.75 | 9602.80 |
| UB6 + sorbitol | 1049.58 | 421.61 | 260052.29 | 31476.86 | 74827.29 | 21027.59 | 15613.54 | 10.02 |
| UB6 + mannitol | 1113.33 | 260.75 | 209047.71 | 40784.74 | 78368.54 | 9137.59 | 26725.21 | 4711.10 |
| UB6 + sucrose | 1330.00 | 169.41 | 43803.33 | 970.80 | 11592.08 | 408.65 | 7183.33 | 1182.34 |
| UB6 + trehalose | 1320.63 | 306.41 | 9674.17 | 1445.15 | 15516.25 | 1373.26 | 9259.17 | 7457.91 |
| UB6 + tween 80 | 11.04 | 7.66 | 671.67 | 707.40 | 185.83 | 219.50 | 231.46 | 139.65 |
| 10 mM acetate/ 125 mM NaCl | 954.58 | 231.28 | 17396.67 | 617.83 | 14698.13 | 1752.45 | 10400.00 | 4.42 |
| water | 18.96 | | 14.79 | | 39.38 | | 49.38 | |

UB6 is 10 mM phosphate, 10 mM citrate pH 6. Water is pure water with no protein. F/T 0 is below freezing.

TABLE 23
Number of particles ≧10μm/mL measured after the listed freeze-thaw cycles.

| | F/T 0 | | F/T 1 | | F/T 2 | | F/T 3 | |
|---|---|---|---|---|---|---|---|---|
| | # >= 10 um | | # >= 10 um | | # >= 10 um | | # >= 10 um | |
| Buffer | ave | sd | ave | sd | ave | sd | ave | sd |
| phosphate | 18.54 | 6.19 | 248.33 | 55.68 | 574.79 | 435.17 | 1224.38 | 408.94 |
| citrate | 26.67 | 12.37 | 533.96 | 383.61 | 968.75 | 192.10 | 899.17 | 217.14 |
| succinate | 138.33 | 128.46 | 359.38 | 149.67 | 908.75 | 207.42 | 1920.21 | 121.98 |
| histidine | 36.25 | 22.98 | 73.33 | 58.04 | 4456.88 | 3766.82 | 6125.21 | 3906.76 |
| arginine | 167.29 | 213.61 | 59.79 | 43.02 | 1113.33 | 500.28 | 2978.54 | 3415.92 |
| Low-ionic | 22.71 | 5.01 | 18.54 | 12.37 | 1871.88 | 2062.10 | 3028.33 | 2162.86 |
| UB6 + sorbitol | 47.50 | 16.50 | 33930.83 | 12385.27 | 8329.79 | 2310.77 | 3302.50 | 1157.00 |
| UB6 + mannitol | 41.25 | 4.12 | 16219.17 | 18940.15 | 7868.13 | 1498.18 | 1774.17 | 969.03 |
| UB6 + sucrose | 43.33 | 1.77 | 3577.29 | 2615.12 | 2069.58 | 991.72 | 1427.92 | 1182.34 |
| UB6 + trehalose | 27.71 | 4.42 | 173.75 | 82.79 | 1866.04 | 27.99 | 379.17 | 310.24 |
| UB6 + tween 80 | 5.42 | 3.54 | 370.63 | 497.92 | 11.04 | 5.01 | 17.50 | 18.56 |
| 10 mM acetate/ | 124.79 | 78.08 | 241.67 | 16.20 | 584.38 | 93.40 | 930.21 | 390.68 |
| 125 mM NaCl | | | | | | | | |
| water | 2.08 | | 1.04 | | 0.00 | | 1.04 | |

UB6 is 10 mM phosphate, 10 mM citrate pH 6. Water is pure water with no protein, low-ionic means the protein is formulated in water without additional excipients added. F/T0 is below freezing.

TABLE 24
Percentage monomer of PG110 samples formulated in various
buffers and excipients after storage during Freeze/Thaw
experiments (SEC data) (UB6 is 10 mM citrate, 10 mM phosphate
pH 6; “low ionic” is the protein in just water).

| | F/T 0 | | F/T 1 | | F/T 2 | | F/T 3 | |
|---|---|---|---|---|---|---|---|---|
| Buffer | ave | sd | ave | sd | ave | sd | ave | sd |
| phosphate | 98.34 | 0.04 | 98.27 | 0.02 | 98.43 | 0.05 | 98.35 | 0.06 |
| citrate | 98.42 | 0.03 | 98.04 | 0.36 | 98.43 | 0.05 | 98.33 | 0.11 |
| succinate | 98.37 | 0.00 | 98.29 | 0.02 | 98.36 | 0.06 | 98.32 | 0.08 |
| histidine | 98.38 | 0.04 | 98.30 | 0.00 | 98.48 | 0.00 | 98.40 | 0.05 |
| arginine | 98.40 | 0.02 | 98.28 | 0.01 | 98.52 | 0.05 | 98.44 | 0.04 |
| Low-ionic | 98.47 | 0.02 | 98.37 | 0.01 | 98.47 | 0.02 | 98.35 | 0.02 |
| UB6 + | 98.40 | 0.04 | 97.87 | 0.05 | 98.04 | 0.04 | 98.05 | 0.03 |
| sorbitol | | | | | | | | |
| UB6 + | 98.41 | 0.00 | 97.34 | 0.07 | 97.47 | 0.02 | 97.43 | 0.09 |
| mannitol | | | | | | | | |
| UB6 + | 98.40 | 0.02 | 98.35 | 0.01 | 98.60 | 0.01 | 98.57 | 0.01 |
| sucrose | | | | | | | | |
| UB6 + | 98.37 | 0.06 | 98.36 | 0.01 | 98.58 | 0.01 | 98.58 | 0.04 |
| trehalose | | | | | | | | |
| UB6 + | 98.23 | 0.04 | 98.26 | 0.04 | 98.55 | 0.02 | 98.57 | 0.02 |
| tween 80 | | | | | | | | |
| 10 mM acetate/ | 98.37 | 0.02 | 98.21 | 0.04 | 98.31 | 0.04 | 98.24 | |
| 125 mM NaCl | | | | | | | | |

TABLE 25
Percentage aggregate of PG110 samples formulated in various
buffers and excipients after storage during Freeze/Thaw
experiments SEC data) (UB6 is 10 mM citrate, 10 mM phosphate
pH 6; low-ionic means the protein is formulated in water
without additional excipients added.)

| | F/T 0 | | F/T 1 | | F/T 2 | | F/T 3 | |
|---|---|---|---|---|---|---|---|---|
| Buffer | ave | sd | ave | sd | ave | sd | ave | sd |
| phosphate | 1.49 | 0.02 | 1.56 | 0.02 | 1.36 | 0.03 | 1.41 | 0.05 |
| citrate | 1.44 | 0.03 | 1.81 | 0.34 | 1.41 | 0.05 | 1.48 | 0.09 |
| succinate | 1.48 | 0.01 | 1.55 | 0.02 | 1.42 | 0.06 | 1.43 | 0.07 |
| histidine | 1.46 | 0.03 | 1.52 | 0.00 | 1.30 | 0.01 | 1.32 | 0.04 |
| arginine | 1.44 | 0.02 | 1.54 | 0.00 | 1.27 | 0.04 | 1.29 | 0.03 |
| Low-ionic | 1.37 | 0.00 | 1.45 | 0.01 | 1.29 | 0.02 | 1.36 | 0.01 |
| UB6 + | 1.47 | 0.04 | 1.98 | 0.04 | 1.77 | 0.04 | 1.74 | 0.03 |
| sorbitol | | | | | | | | |
| UB6 + | 1.45 | 0.00 | 2.50 | 0.05 | 2.33 | 0.03 | 2.37 | 0.09 |
| mannitol | | | | | | | | |
| UB6 + | 1.46 | 0.01 | 1.48 | 0.01 | 1.23 | 0.01 | 1.23 | 0.02 |
| sucrose | | | | | | | | |
| UB6 + | 1.48 | 0.06 | 1.48 | 0.01 | 1.22 | 0.01 | 1.22 | 0.03 |
| trehalose | | | | | | | | |
| UB6 + | 1.61 | 0.04 | 1.56 | 0.04 | 1.24 | 0.00 | 1.21 | 0.01 |
| tween 80 | | | | | | | | |
| 10 mM acetate/ | 1.46 | 0.02 | 1.60 | 0.04 | 1.44 | 0.04 | 1.49 | |
| 125 mM NaCl | | | | | | | | |

TABLE 26
Percentage fragment of PG110 samples formulated in various
buffers and excipients after storage during Freeze/Thaw
experiments (SEC data) (UB6 is 10 mM citrate, 10 mM phosphate
pH 6; low-ionic means the protein is formulated in water
without additional excipients added)

| | F/T 0 | | F/T 1 | | F/T 2 | | F/T 3 | |
|---|---|---|---|---|---|---|---|---|
| Buffer | ave | sd | ave | sd | ave | sd | ave | sd |
| phosphate | 0.17 | 0.02 | 0.18 | 0.00 | 0.20 | 0.03 | 0.24 | 0.01 |
| citrate | 0.14 | 0.00 | 0.15 | 0.02 | 0.17 | 0.00 | 0.20 | 0.01 |
| succinate | 0.15 | 0.00 | 0.16 | 0.00 | 0.22 | 0.00 | 0.25 | 0.01 |
| histidine | 0.15 | 0.02 | 0.18 | 0.00 | 0.21 | 0.00 | 0.28 | 0.01 |
| arginine | 0.15 | 0.00 | 0.18 | 0.00 | 0.20 | 0.01 | 0.27 | 0.01 |
| Low-ionic | 0.16 | 0.02 | 0.18 | 0.00 | 0.24 | 0.01 | 0.28 | 0.01 |
| UB6 + | 0.14 | 0.00 | 0.15 | 0.00 | 0.19 | 0.00 | 0.21 | 0.00 |
| sorbitol | | | | | | | | |
| UB6 + | 0.13 | 0.01 | 0.16 | 0.01 | 0.20 | 0.01 | 0.20 | 0.00 |
| mannitol | | | | | | | | |
| UB6 + | 0.15 | 0.01 | 0.16 | 0.00 | 0.18 | 0.00 | 0.20 | 0.01 |
| sucrose | | | | | | | | |
| UB6 + | 0.14 | 0.00 | 0.16 | 0.01 | 0.19 | 0.00 | 0.20 | 0.01 |
| trehalose | | | | | | | | |
| UB6 + | 0.16 | 0.00 | 0.18 | 0.00 | 0.21 | 0.01 | 0.23 | 0.01 |
| tween 80 | | | | | | | | |
| 10 mM acetate/ | 0.17 | 0.00 | 0.19 | 0.00 | 0.25 | 0.00 | 0.27 | |
| 125 mM NaCl | | | | | | | | |

### Example 8

### Impact of Buffers and Excipients on the Physico-Chemical Stability of PG110 Formulations During Accelerated Stability Testing

Earlier examples have discussed the factors affecting the stability of PG110 formulations during long-term storage, including pH and storage temperature. In addition to these extrinsic factors, the formulation ingredients themselves must be evaluated for their impact on protein drug substance stability during storage. In order to carry this out, the protein drug substance is exposed to short-term storage at elevated temperatures during preformulation and formulation project stages in order to quickly gain insight in the formulation feasibility for long-term storage at lower temperatures (in most cases 2-8° C.).

Storage stability of the PG110 antibody in solution was evaluated at various temperatures for prolonged periods of time at controlled temperature conditions at pH 6 in different buffers and excipients. The condition of pH 6 was chosen based upon prior experiments which determined that it was near the optimal pH for chemical and physical stability. After defined storage periods, samples were pulled and the impact of storage time and storage temperature on PG110 stability was evaluated by SEC and iCIEF.

In this study, PG110 was formulated at 2 mg/ml in various buffers and excipients listed in Table 27.

TABLE 27
Buffers and excipients tested for their affect on the physical and
chemical stability of PG110 subjected to storage at elevated temperatures
(universal buffer is 10 mM citrate, 10 mM phosphate pH 6)

15 mM sodium phosphate
15 mM sodium citrate
15 mM sodium succinate
15 mM sodium acetate
15 mM arginine
15 mM histidine
Low-ionic formulation
10 mM universal buffer and 40 mg/mL mannitol
10 mM universal buffer and 40 mg/mL sorbitol
10 mM universal buffer and 80 mg/mL sucrose
10 mM universal buffer and 80 mg/mL trehalose
10 mM universal buffer and 2.5% (m/m) glycerol
10 mM universal buffer and 15 mM ammonium sulfate
10 mM universal buffer and 20 mM sodium chloride
10 mM universal buffer and 200 mM sodium chloride
10 mM universal buffer and 0.01% (m/m) polysorbate 80
10 mM universal buffer and 0.01% (m/m) polysorbate 20
10 mM universal buffer and 0.1% (m/m) poloxamer 188

Samples were then stored under controlled conditions (in temperature chambers and in the absence of light) at various temperatures. At predefined points of time, samples of prepared solutions were pulled for analysis according to the sample pull scheme provided in Table 28 and 29 for SEC and iCIEF, respectively. Numbers refer to number of vials that were stored/pulled for each buffer or excipients condition. Data is provided in Tables 30, 31 and 32.

TABLE 28
Sample Pull Scheme

| | T0 | 7 days | 6 months | 12 months |
|---|---|---|---|---|
| 5° C. | | | | 1 |
| 25° C. | | | 1 | |
| 40° C. | 1 | 1 | | |
| 50° C. | | 1 | | |

TABLE 29
Sample Pull Scheme

| | T0 | 4 months | 12 months |
|---|---|---|---|
| 5° C. | | 1 | 1 |
| 25° C. | | 1 | |
| 40° C. | 1 | 1 | |

TABLE 30
Percentage monomer of PG110 samples formulated in various buffers and
excipients after storage at specified temperatures and times (SEC data)
(UB6 is 10 mM citrate, 10 mM phosphate pH 6; low-ionic means the protein
is formulated in water without additional excipients added)

| Buffer | T0 | T7 d 50° C. | T7 d 40° C. | 6 month 25° C. | 12 month 5° C. |
|---|---|---|---|---|---|
| 15 mM phosphate | 98.38 | 93.84 | 97.92 | 94.73 | 93.84 |
| 15 mM acetate | 98.45 | 94.19 | 97.92 | 95.41 | 97.46 |
| 15 mM citrate | 98.35 | 93.92 | 98.03 | 95.35 | 97.49 |
| 15 mM succinate | 98.44 | 94.08 | 98.08 | 95.08 | 97.4 |
| 15 mM histidine | 98.58 | 94.25 | 98.2 | 95.29 | 97.59 |
| 15 mM arginine | 98.5 | 93.17 | 98.16 | 96.37 | 97.79 |
| Low-ionic | 98.85 | 95.88 | 98.69 | 96.37 | 98.02 |
| UB6 + 4% sorbitol | 98.16 | 94.86 | 98.11 | 95.5 | 97.4 |
| UB6 + 4% mannitol | 98.05 | 94.91 | 98.05 | 95.34 | 97.45 |
| UB6 + 8% sucrose | 98.52 | 95.08 | 98.09 | 95.77 | 97.5 |
| UB6 + 8% trehalose | 98.45 | 94.96 | 98.06 | 95.33 | 97.3 |
| UB6 + 0.01% Tween 80 | 98.43 | 93.98 | 97.88 | 92.78 | 96.76 |
| UB6 + 2.5% glycerol | 98.53 | — | 97.74 | 95.2 | 97.24 |
| UB6 + 15 mM (NH4)2SO4 | 98.35 | 94.79 | 98.07 | 95.17 | 97.26 |
| UB6 + 20 mM NaCl | 98.39 | 94.42 | 98 | 95.26 | 97.26 |
| UB6 + 200 mM NaCl | 98.4 | 94.58 | 98.12 | 95.04 | 97.04 |
| UB6 + 0.01% Tween 20 | 98.42 | 94.08 | 97.84 | 94.66 | 97.23 |
| UB6 + 0.1% Poloxamer | 98.47 | 94.54 | 97.98 | 95.07 | 97.16 |

TABLE 31
Percentage aggregate of PG110 samples formulated in various buffers and excipients after storage
at specified temperatures and times (SEC data) (UB6 is 10 mM citrate, 10 mM phosphate pH 6;
low-ionic means the protein is formulated in water without additional excipients added).

| Buffer | T0 | T7 d 50° C. | T7 d 40° C. | 6 month 25° C. | 12 months 5° C. |
|---|---|---|---|---|---|
| 15 mM phosphate | 1.42 | 3.45 | 1.87 | 2.77 | 5.22 |
| 15 mM acetate | 1.34 | 3.47 | 1.88 | 2.32 | 1.65 |
| 15 mM citrate | 1.48 | 3.47 | 1.78 | 2.34 | 1.6 |
| 15 mM succinate | 1.35 | 3.31 | 1.73 | 2.61 | 1.66 |
| 15 mM histidine | 1.22 | 3.51 | 1.6 | 2.29 | 1.5 |
| 15 mM arginine | 1.3 | 4.54 | 1.63 | 1.73 | 1.39 |
| Low-ionic | 0.95 | 1.53 | 1.07 | 1.61 | 1.01 |
| UB6 + 4% sorbitol | 1.67 | 3.13 | 1.71 | 2.16 | 1.63 |
| UB6 + 4% mannitol | 1.77 | 2.91 | 1.76 | 2.28 | 1.63 |
| UB6 + 8% sucrose | 1.31 | 2.76 | 1.73 | 1.98 | 1.59 |
| UB6 + 8% trehalose | 1.37 | 3 | 1.75 | 2.4 | 1.65 |
| UB6 + 0.01% Tween 80 | 1.38 | 3.55 | 1.93 | 4.75 | 1.88 |
| UB6 + 2.5% glycerol | 1.29 | — | 2.04 | 2.42 | 1.69 |
| UB6 + 15 mM (NH4)2SO4 | 1.49 | 3.22 | 1.74 | 2.44 | 1.68 |
| UB6 + 20 mM NaCl | 1.43 | 3.33 | 1.81 | 2.26 | 1.63 |
| UB6 + 200 mM NaCl | 1.42 | 3.28 | 1.69 | 2.47 | 1.93 |
| UB6 + 0.01% Tween 20 | 1.39 | 3.81 | 1.95 | 2.78 | 1.82 |
| UB6 + 0.1% Poloxamer | 1.34 | 3.32 | 1.83 | 2.49 | 1.76 |

TABLE 32
Percentage fragment of PG110 samples formulated in various buffers and
excipients after storage at specified temperatures and times (SEC data)
(UB6 is 10 mM citrate, 10 mM phosphate pH 6; low-ionic means the protein
is formulated in water without additional excipients added).

| Buffer | T0 | T7 d 50° C. | T7 d 40° C. | 6 month 25° C. | 12 month 5° C. |
|---|---|---|---|---|---|
| 15 mM phosphate | 0.20 | 2.71 | 0.2 | 2.49 | 0.93 |
| 15 mM acetate | 0.21 | 2.34 | 0.20 | 2.25 | 0.88 |
| 15 mM citrate | 0.17 | 2.61 | 0.19 | 2.29 | 0.9 |
| 15 mM succinate | 0.21 | 2.61 | 0.19 | 2.29 | 0.93 |
| 15 mM histidine | 0.19 | 2.25 | 0.19 | 2.41 | 0.90 |
| 15 mM arginine | 0.20 | 2.28 | 0.21 | 1.89 | 0.81 |
| Low-ionic | 0.20 | 2.59 | 0.24 | 2.52 | 0.95 |
| UB6 + 4% sorbitol | 0.17 | 2.01 | 0.18 | 2.33 | 0.96 |
| UB6 + 4% mannitol | 0.18 | 2.18 | 0.19 | 2.36 | 0.91 |
| UB6 + 8% sucrose | 0.17 | 2.16 | 0.19 | 2.23 | 0.89 |
| UB6 + 8% trehalose | 0.18 | 2.04 | 0.19 | 2.25 | 1.04 |
| UB6 + 0.01% Tween 80 | 0.19 | 2.46 | 0.20 | 2.45 | 1.34 |
| UB6 + 2.5% glycerol | 0.18 | — | 0.21 | 2.36 | 1.06 |
| UB6 + 15 mM (NH4)2SO4 | 0.17 | 2 | 0.19 | 2.37 | 1.05 |
| UB6 + 20 mM NaCl | 0.17 | 2.25 | 0.19 | 2.46 | 1.09 |
| UB6 + 200 mM NaCl | 0.18 | 2.15 | 0.19 | 2.47 | 1.02 |
| UB6 + 0.01% Tween 20 | 0.19 | 2.11 | 0.20 | 2.54 | 0.94 |
| UB6 + 0.1% Poloxamer | 0.18 | 2.14 | 0.19 | 2.42 | 1.07 |

TABLE 33
Percentage of various species of PG110 samples formulated
in various buffers and excipients after storage at specified
temperatures and times (iCIEF data) (UB6 is 10 mM citrate,
10 mM phosphate pH 6; low-ionic means the protein is formulated
in water without additional excipients added).

| 4 month various | | | | | | |
|---|---|---|---|---|---|---|
| temperatures | | | Area % | | | |
| Buffer | | | Acidic | Main | Basic | pI |
| 15 mM Phosphate | 5° | C. | 33.74 | 57.22 | 9.05 | 6.94 |
| | 25° | C. | 43.27 | 48.69 | 8.04 | 6.93 |
| | 40° | C. | 86.30 | 8.26 | 5.44 | 6.91 |
| 15 mM Acetate | 5° | C. | 33.59 | 57.03 | 9.39 | 6.93 |
| | 25° | C. | 43.25 | 48.71 | 8.04 | 6.93 |
| | 40° | C. | 86.02 | 10.89 | 3.09 | 6.91 |
| 15 mM Citrate | 5° | C. | 33.05 | 57.54 | 9.41 | 6.96 |
| | 25° | C. | 41.94 | 50.06 | 8.00 | 6.94 |
| | 40° | C. | 85.38 | 11.27 | 3.35 | 6.93 |
| 15 mM Succinate | 5° | C. | 32.62 | 58.43 | 8.95 | 6.96 |
| | 25° | C. | 41.29 | 50.55 | 8.16 | 6.96 |
| | 40° | C. | 83.17 | 13.98 | 2.85 | 6.94 |
| 15 mM Histidine | 5° | C. | 32.79 | 57.99 | 9.22 | 6.99 |
| | 25° | C. | 37.97 | 52.85 | 9.18 | 6.99 |
| | 40° | C. | 70.99 | 21.43 | 7.59 | 6.98 |
| 15 mM Arginine | 5° | C. | 31.47 | 55.43 | 13.11 | 6.82 |
| | 25° | C. | 39.09 | 49.00 | 11.91 | 6.80 |
| | 40° | C. | 92.62 | 5.66 | 1.72 | 6.85 |
| Low Ionic | 5° | C. | 33.48 | 57.45 | 9.07 | 7.00 |
| | 25° | C. | 39.22 | 51.41 | 9.37 | 7.00 |
| | 40° | C. | 74.02 | 19.15 | 6.83 | 6.99 |
| UB6 + 4% Sorbitol | 5° | C. | 32.63 | 58.32 | 9.05 | 6.92 |
| | 25° | C. | 42.57 | 49.25 | 8.18 | 6.92 |
| | 40° | C. | 93.73 | 2.96 | 3.31 | 6.92 |
| UB6 + 4% Mannitol | 5° | C. | 33.92 | 56.96 | 9.13 | 6.93 |
| | 25° | C. | 46.64 | 45.87 | 7.49 | 6.92 |
| | 40° | C. | 88.15 | 9.80 | 2.05 | 6.89 |
| UB6 + 8% Sucrose | 5° | C. | 33.19 | 57.64 | 9.17 | 6.90 |
| | 25° | C. | 43.62 | 48.52 | 7.86 | 6.88 |
| | 40° | C. | 87.02 | 11.04 | 1.94 | 6.85 |
| UB6 + 8% Trehalose | 5° | C. | 34.44 | 56.30 | 9.26 | 6.94 |
| | 25° | C. | 48.41 | 44.27 | 7.33 | 6.93 |
| | 40° | C. | 89.82 | 6.74 | 3.44 | 6.89 |
| UB6 + 0.01% Tween80 | 5° | C. | 36.81 | 54.24 | 8.95 | 6.94 |
| | 25° | C. | 90.56 | 7.36 | 2.08 | 6.93 |
| | 40° | C. | 100.00 | 0.00 | 0.00 | NA |
| UB6 + 2.5% glycerol | 5° | C. | 33.21 | 57.40 | 9.39 | 6.95 |
| | 25° | C. | 41.22 | 50.31 | 8.47 | 6.94 |
| | 40° | C. | 81.69 | 13.58 | 4.73 | 6.92 |
| UB6 + 15 mM (NH4)2SO4 | 5° | C. | 33.10 | 57.65 | 9.25 | 6.93 |
| | 25° | C. | 42.47 | 49.07 | 8.46 | 6.92 |
| | 40° | C. | 84.46 | 12.13 | 3.41 | 6.89 |
| UB6 + 20 mM NaCl | 5° | C. | 41.19 | 48.50 | 10.31 | 6.81 |
| | 25° | C. | 47.99 | 42.16 | 9.85 | 6.79 |
| | 40° | C. | 84.56 | 12.51 | 2.93 | 6.77 |
| UB6 + 200 mM NaCl | 5° | C. | 33.13 | 58.03 | 8.84 | 6.92 |
| | 25° | C. | 43.32 | 48.12 | 8.56 | 6.92 |
| | 40° | C. | 84.26 | 10.55 | 5.19 | 6.91 |
| UB6 + 0.01% Tween 20 | 5° | C. | 33.71 | 57.18 | 9.11 | 6.93 |
| | 25° | C. | 43.57 | 48.18 | 8.25 | 6.93 |
| | 40° | C. | 84.79 | 12.37 | 2.84 | 6.90 |
| UB6 + 0.1% Poloxamer | 5° | C. | 34.02 | 56.77 | 9.21 | 6.93 |
| | 25° | C. | 42.82 | 49.11 | 8.07 | 6.93 |
| | 40° | C. | 84.80 | 12.90 | 2.30 | 6.90 |

| 12 months 5° C. | | | | | | |
|---|---|---|---|---|---|---|
| buffer | | | Acidic | Main | Basic | pI value |
| 15 mM phosphate | 5° | C. | 38.194 | 53.23 | 8.576 | 7.12 |
| 15 mM acetate | 5° | C. | 38.226 | 53.21 | 8.564 | 7.09 |
| 15 mM citrate | 5° | C. | 37.955 | 53.81 | 8.235 | 7.14 |
| 15 mM succinate | 5° | C. | 37.633 | 53.88 | 8.487 | 7.16 |
| 15 mM histidine | 5° | C. | 36.612 | 54.71 | 8.678 | 7.13 |
| 15 mM arginine | 5° | C. | 36.923 | 52.25 | 10.827 | 6.99 |
| Low-ionic | 5° | C. | 36.949 | 54.71 | 8.341 | 7.23 |
| UB6 + 4% sorbitol | 5° | C. | 38.342 | 53.06 | 8.598 | 7.11 |
| UB6 + 4% mannitol | 5° | C. | 39.883 | 51.58 | 8.537 | 7.11 |
| UB6 + 8% sucrose | 5° | C. | 38.023 | 53.11 | 8.867 | 7.09 |
| UB6 + 8% trehalose | 5° | C. | 38.92 | 52.75 | 8.33 | 7.11 |
| UB6 + 0.01% Tween 80 | 5° | C. | 48.371 | 44.8 | 6.829 | 7.12 |
| UB6 + 2.5% glycerol | 5° | C. | 37.078 | 54.34 | 8.582 | 7.12 |
| UB6 + 15 mM (NH4)2SO4 | 5° | C. | 38.121 | 53.54 | 8.339 | 7.08 |
| UB6 + 20 mM NaCl | 5° | C. | 42.232 | 49.73 | 8.038 | 6.9 |
| UB6 + 200 mM NaCl | 5° | C. | 38.253 | 53.45 | 8.297 | 7.14 |
| UB6 + 0.01% Tween 20 | 5° | C. | 37.619 | 53.97 | 8.411 | 7.14 |
| UB6 + 0.1% Poloxamer | 5° | C. | 38.145 | 53.13 | 8.725 | 7.15 |

PG110 stability decreased with increasing storage temperature which is expected behavior for all proteins. However, the data collected thus far indicate that formulating PG110 using phosphate, arginine or glycerol would result in potential denaturation. After 50° C. storage for 7 days with glycerol, no protein was detected via SEC, indicating all PG110 has been undergone physical instability and insoluble aggregate formation, thus avoiding SEC/UV detection.

### Example 9

### Impact of Buffers and Formulation Excipients on the Stability of PG110 Formulations Stored at −80° C.

Findings from prior examples led to the decision that a formulation of 15 mM histidine and 0.01% tween 80 was optimal for the prevention of visible particle formation in liquid formulations of the drug substance (Example 6). Tween 80 also prevented the formation of subvisible particles induced by freeze-thaw stress as detailed in Example 7. Accelerated stability testing (Example 8) also determined that the two excipients did not cause unacceptable levels of aggregation or fragmentation.

With this in mind, the next concern is whether the excipients cause destabilization of the drug substance when stored at −80° C. To test this, 150 μl solutions of PG110 at 1 mg/ml and 10 mg/ml in the original formulation (10 mM acetate 125 mM NaCl), 15 mM histidine pH 6, and 15 mM histidine pH 6+0.01% Tween 80 were prepped and stored at −80° C. in cryovials. At 5 days, vials of each sample were removed from storage and physicochemical degradation was quantitated by SEC. At 10 days, the remaining vial of each sample were removed and analyzed the same way. Tables 34, 35, and 36 contain the results of these experiments.

The data show that for formulations with histidine or histidine+tween 80 the % monomer increases from 0 to 5 days and remains at that level at least until 10 days. In contrast, PG110 at 10 mg/ml in 10 mM acetate and 125 mM NaCl shows a steady decrease in % monomer from 0 to 5 days to 10 days which corresponds to an increase in % aggregate. Overall, the data indicate that a histidine+tween 80 formulation does not destabilize the drug substance when stored at −80° C.

TABLE 34
Percentage monomer of PG110 samples formulated in various buffers
and excipients after storage at −80° C. (SEC data).

| | T0 | T5 d −80° C. | | T10 d −80° C. | |
|---|---|---|---|---|---|
| Buffer | ave | ave | sd | ave | sd |
| 1 mg/ml 10 mM acetate 125 mM | 98.24 | 98.31 | 0.01 | 98.38 | 0.02 |
| NaCl | | | | | |
| 10 mg/ml 10 mM acetate 125 mM | 98.24 | 97.99 | 0.01 | 97.77 | 0.02 |
| NaCl | | | | | |
| 1 mg/ml histidine pH 6 | 98.22 | 98.38 | 0.07 | 98.42 | 0.03 |
| 10 mg/ml histidine pH 6 | 98.22 | 98.47 | 0.01 | 98.44 | 0.02 |
| 1 mg/ml histidine pH 6 + | 98.16 | 98.43 | 0.01 | 98.38 | 0.02 |
| 0.01% tween80 | | | | | |
| 10 mg/ml histidine pH 6 + | 98.16 | 98.45 | 0.01 | 98.43 | 0.01 |
| 0.01% tween80 | | | | | |

TABLE 35
Percentage aggregate of PG110 samples formulated in various buffers
and excipients after storage at −80° C. (SEC data).

| | T0 | T5 d −80° C. | | T10 d −80° C. | |
|---|---|---|---|---|---|
| Buffer | ave | ave | sd | ave | sd |
| 1 mg/ml 10 mM acetate 125 mM | 1.58 | 1.50 | 0.01 | 1.33 | 0.00 |
| NaCl | | | | | |
| 10 mg/ml 10 mM acetate 125 mM | 1.58 | 1.81 | 0.01 | 1.92 | 0.02 |
| NaCl | | | | | |
| 1 mg/ml histidine pH 6 | 1.59 | 1.42 | 0.06 | 1.29 | 0.01 |
| 10 mg/ml histidine pH 6 | 1.59 | 1.33 | 0.00 | 1.28 | 0.01 |
| 1 mg/ml histidine pH 6 + | 1.65 | 1.35 | 0.01 | 1.31 | 0.00 |
| 0.01% tween80 | | | | | |
| 10 mg/ml histidine pH 6 + | 1.65 | 1.33 | 0.00 | 1.24 | 0.00 |
| 0.01% tween80 | | | | | |

TABLE 36
Percentage fragment of PG110 samples formulated in various buffers
and excipients after storage at −80° C. (SEC data).

| | T0 | T5 d −80° C. | | T10 d −80° C. | |
|---|---|---|---|---|---|
| Buffer | ave | ave | sd | ave | sd |
| 1 mg/ml 10 mM acetate 125 mM | 0.18 | 0.19 | 0.00 | 0.29 | 0.02 |
| NaCl | | | | | |
| 10 mg/ml 10 mM acetate 125 mM | 0.18 | 0.20 | 0.00 | 0.31 | 0.00 |
| NaCl | | | | | |
| 1 mg/ml histidine pH 6 | 0.19 | 0.19 | 0.01 | 0.30 | 0.02 |
| 10 mg/ml histidine pH 6 | 0.19 | 0.19 | 0.01 | 0.29 | 0.01 |
| 1 mg/ml histidine pH 6 + | 0.19 | 0.22 | 0.00 | 0.32 | 0.02 |
| 0.01% tween80 | | | | | |
| 10 mg/ml histidine pH 6 + | 0.19 | 0.21 | 0.01 | 0.34 | 0.01 |
| 0.01% tween80 | | | | | |

The visual inspection data also showed that the histidine containing formulations even at 100 mg/mL did not contain visible particle formation even after 4 F/T cycles, further indicating that histidine is a very suitable excipient for maintaining PG110 stability.

### Example 10

### Impact of Freeze-Thawing, Stirring and Accelerated Stability Testing on the Stability of PG110 in Various Formulations at Various Concentrations

The impact of excipients on PG110 stability was evaluated in various stress experiments:
1) repeated freeze-thaw processing (−80° C./30° C. water bath);
2) Stirring to effectively exert stir stress and to increase the air-liquid interface to induce physical instability and PG110 degradation (6R glass vial, ambient temperature, approx. 9 mm Teflon coated stir bar, 550 rpm, up to 48 hrs stirring);
3) Accelerated stability testing: various samples were put on real-time and accelerated stability at 2-5 C, 25° C./60% relH and 40° C./60% relH, and the impact of protein concentration and stabilizing excipients on the content of native PG110 monomer was monitored by SEC/UV.
   The following PG110 formulations and formulation compositions were tested:
   Formulation 1: 52 mg/mL PG110, pH 6.0;
   2.33 mg/mL Histidine;
   5.0 mg/mL Sucrose;
   20.0 mg/mL Mannitol; and
   0.10 mg/mL Polysorbate 80.
   Formulation 2: 52 mg/mL PG110, pH 6.0;
   2.33 mg/mL Histidine;
   46 mg/mL Sucrose; and
   0.10 mg/mL Polysorbate 80.
   Formulation 3: 52 mg/mL PG110, pH 6.0.;
   2.33 mg/mL Histidine;
   46 mg/mL Trehalose; and
   0.10 mg/mL Polysorbate 80.
   Formulation 4: 20 mg/mL PG110, pH 6.0;
   2.33 mg/mL Histidine;
   5.0 mg/mL Sucrose;
   20.0 mg/mL Mannitol; and
   0.10 mg/mL Polysorbate 80.
   Formulation 5: 20 mg/mL PG110, pH 6.0;
   2.33 mg/mL Histidine;
   46 mg/mL Sucrose; and
   0.10 mg/mL Polysorbate 80.
   Formulation 6: 20 mg/mL PG110, pH 6.0;
   2.33 mg/mL Histidine;
   46 mg/mL Trehalose; and
   0.10 mg/mL Polysorbate 80.

Freeze thaw stability of the ABT110 antibody at protein concentrations of 52 mg/mL and 20 mg/mL, were as follows after 2 and after 4 f/t cycles, respectively.

TABLE 37
Monomer content as determined by SEC/UV

| | Form | Form | Form | Form | Form | Form |
|---|---|---|---|---|---|---|
| | #1 | #2 | #3 | #4 | #5 | #6 |
| 0 f/t | 98.20 | 98.19 | 98.19 | 97.94 | 98.07 | 98.33 |
| 2 f/t | 98.22 | 98.21 | 98.20 | 98.07 | 98.19 | 98.31 |
| 4 f/t | 98.19 | 98.18 | 98.17 | 98.10 | 98.22 | 98.30 |

The foregoing data demonstrate that sucrose, trehalose and mannitol are well suited to maintain physical stability of PG110 during repeated f/t stress. Virtually no degradation was detected with regard to native PG110 monomer content throughout the stress experiment.

Stir stress stability of the ABT110 antibody at protein concentrations of 52 mg/mL and 20 mg/mL, were as follows after 24 and 48 hrs of stirring, respectively.

TABLE 38
Monomer content as determined by SEC/UV

| | Form | Form | Form | Form | Form | Form |
|---|---|---|---|---|---|---|
| | #1 | #2 | #3 | #4 | #5 | #6 |
| 0 hrs | 98.20 | 98.19 | 98.18 | 97.94 | 98.07 | 98.33 |
| 24 hrs | 98.24 | 98.22 | 98.24 | 98.00 | 98.15 | 98.38 |
| 48 hrs | 98.20 | 98.21 | 98.20 | 97.96 | 98.08 | 98.36 |

The foregoing data demonstrate that sucrose, trehalose and mannitol are well suited to maintain physical stability of PG110 during extensive stir stress. Virtually no degradation was detected with regard to native PG110 monomer content throughout the stress experiment.

Accelerated degradation kinetics of the ABT110 antibody at protein concentrations of 52 mg/mL and 20 mg/mL, were as follows after 14 days at 5° C. and after 14 days at 50° C.

TABLE 39
Monomer content as determined by SEC/UV

| | Form | Form | Form | Form | Form | Form |
|---|---|---|---|---|---|---|
| | #1 | #2 | #3 | #4 | #5 | #6 |
| 0 hrs | 98.20 | 98.19 | 98.19 | 97.94 | 98.07 | 98.33 |
| 14 d, 5° C. | 98.19 | 98.11 | 98.21 | 97.92 | 97.97 | 98.30 |
| 14 d, 50° C. | 85.16 | 85.09 | 85.29 | 84.82 | 85.04 | 84.82 |

The foregoing data demonstrate that sucrose, trehalose and mannitol are well suited to maintain physical stability of PG110 during longer term storage. Even when exposed to 50° C. for 14 days, more than 80% of native monomer was present in all samples tested.

### Example 11

### Long Term Stability of PG110 Lyophilized Powder Stored Under Various Conditions

The suitability of sucrose and mannitol as stabilizers during lyophilization and storage of PG110 was further studied. Two formulations of PG110 lyophilized powder for injection solution were placed under longer-term storage conditions (2-8° C.), accelerated storage conditions of 25°/60% RH, and stress conditions of 40° C./75% RH and 50° C. These laboratory-scale drug product batches were produced and lyophilized from 130 L scale drug substance manufactured according to standard methods, for example, as shown in Table 40.

TABLE 40
Lyophilization Conditions for Formulations 1 and 2

| Program Step | Shelf Temp [±2° C.] | Pressure [mbar] | Time [h:min] |
|---|---|---|---|
| Loading | +20° C. | Atm. | — |
| Freezing | +20° to 0° C. | Atm. | 0:20 |
| | 0° C. | Atm. | 2:10 |
| | 0° C. to −45° C. | Atm. | 2:30 |
| | −45° C. | Atm. | 3:00 |
| Primary drying | −45° C. | 0.66 ± 0.01 | 1:00 |
| | −45° C. to −25° C. | 0.66 ± 0.01 | 1:00 |
| | −25° C. | 0.66 ± 0.01 | 90:00 |
| Secondary Drying | −25° C. | 0.36 ± 0.01 | 01:00 |
| | −25° C. to +25° C. | 0.36 ± 0.01 | 04:30 |
| | +25° C. | 0.36 ± 0.01 | 08:00 |
| Holding Step | +25° C. to +5° C. | 0.36 ± 0.01 | 0:30 |
| | +5° C. | 0.36 ± 0.01 | — |
| Pre-aeration | +5° C. | About 500 | — |
| and closing | | | |
| Aeration | +5° C. | Atm. | — |

For testing, samples of the formulations were resuspended in sterile, distilled water at room temperature.

Formulation 1:
20 mg/mL PG110, pH 5.5
2.33 mg/mL histidine
70 mg/mL sucrose
0.1 mg/mL polysorbate 80
Formulation 2:
20 mg/mL PG110, pH 5.5
2.33 mg/mL histidine
10 mg/mL sucrose
30 mg/mL mannitol
0.1 mg/mL polysorbate 80

Test methods related to the quality, biological activity, and purity of the drug substance were performed at various time points to assess the stability profile of PG110 in each batch. The analytical methods used included:
Appearance (visual)
Particles (visual)
Subvisible particles (light blockade)
pH
Imaged capillary isoelectric focusing (icIEF)
SDS PAGE (reduced and nonreduced)
Size Exclusion HPLC
Product Specific Antigen Binding Assay
Product Specific Functional Bioassay

Container closure integrity testing was performed using a dye penetration method in which the drug product vial was exposed to vacuum in a methylene blue solution, and then visually inspected for blue coloration. Water content was determined per USP, according to standard methods. Stability data obtained for samples from batch 1 and batch 2 are provided in Tables 41-48.

TABLE 41
Stability of PG110 Lyophilized Formulation 1 Stored at 2-8° C.

| Test | Procedure | Characteristics | Initial | 1 month | 3 months | 6 months |
|---|---|---|---|---|---|---|
| Size exclusion HPLC | HPLC | Aggregates [%] | 1.6 | 1.7 | 1.6 | 1.6 |
| (SE-HPLC) | | Fragments [%] | <0.1 | <0.1 | <0.1 | <0.1 |
| | | Monomer [%] | 98.3 | 98.3 | 98.3 | 98.4 |
| Capillary isoelectric | Isoelectric | Sum acidic region [%] | 33.7 | 33.3 | 32.8 | 32.5 |
| focusing (icIEF) | focusing | Main Peak [%] | 56.2 | 57.1 | 57.3 | 58.9 |
| | | Sum basic region [%] | 10.1 | 9.7 | 9.9 | 8.6 |
| SDS gel | SDS-Page (R) | Purity [%] | 99.6 | 99.5 | 99.6 | 99.7 |
| electrophoresis (SDS- | | | | | | |
| PAGE reducing) | | | | | | |
| SDS gel | SDS-Page (NR) | Purity [%] | 92.3 | 92.2 | 91.8 | 90.6 |
| electrophoresis (SDS- | | Band present at 97 kDa [%] | 0.3 | 0.3 | 0.3 | 0.4 |
| PAGE non-reducing) | | | | | | |
| Particulate | Subvis. Part. | Particles ≧10 μm | 30 | 7 | 19 | 14 |
| contamination - Sub- | (LO) | [/container] | | | | |
| visible Particles | | Particles ≧25 μm | 1 | 0 | 1 | 2 |
| | | [/container] | | | | |

TABLE 42
Stability of PG110 Lyophilized Formulation 1 Stored at 25° C./60% RH

| Test | Procedure | Characteristics | Initial | 3 months | 6 months |
|---|---|---|---|---|---|
| Size exclusion HPLC | HPLC | Aggregates [%] | 1.6 | 1.8 | 1.6 |
| (SE-HPLC) | | Fragments [%] | <0.1 | <0.1 | <0.1 |
| | | Monomer [%] | 98.3 | 98.2 | 98.4 |
| Capillary isoelectric | Isoelectric focusing | Sum acidic region [%] | 33.7 | 34.0 | 31.7 |
| focusing (icIEF) | | Main Peak [%] | 56.2 | 57.2 | 59.2 |
| | | Sum basic region [%] | 10.1 | 8.8 | 9.1 |
| SDS gel electrophoresis | SDS-Page (R) | Purity [%] | 99.6 | 99.6 | 99.8 |
| (SDS-PAGE reducing) | | | | | |
| SDS gel electrophoresis | SDS-Page (NR) | Purity [%] | 92.3 | 92.1 | 90.6 |
| (SDS-PAGE non- | | Band present at 97 kDa [%] | 0.3 | 0.4 | 0.3 |
| reducing) | | | | | |
| Particulate | Subvis. Part. (LO) | Particles ≧10 μm [/container] | 30 | 35 | 30 |
| contamination - Sub- | | Particles ≧25 μm [/container] | 1 | 1 | 1 |
| visible Particles | | | | | |

TABLE 43
Stability of PG110 Lyophilized Formulation 1 Stored at 40° C./75% RH

| Test | Procedure | Characteristics | Initial | 1 month | 3 months | 6 months |
|---|---|---|---|---|---|---|
| Size exclusion HPLC | HPLC | Aggregates [%] | 1.6 | 1.7 | 1.7 | 1.7 |
| (SE-HPLC) | | Fragments [%] | <0.1 | <0.1 | <0.1 | <0.1 |
| | | Monomer [%] | 98.3 | 98.3 | 98.2 | 98.3 |
| Capillary isoelectric | Isoelectric | Sum acidic region [%] | 33.7 | 33.6 | 33.1 | 32.5 |
| focusing (icIEF) | focusing | Main Peak [%] | 56.2 | 56.4 | 55.9 | 55.1 |
| | | Sum basic region [%] | 10.1 | 10.0 | 11.0 | 12.3 |
| SDS gel | SDS-Page (R) | Purity [%] | 99.6 | 99.6 | 99.5 | 99.7 |
| electrophoresis (SDS- | | | | | | |
| PAGE reducing) | | | | | | |
| SDS gel | SDS-Page (NR) | Purity [%] | 92.3 | 92.6 | 91.4 | 90.9 |
| electrophoresis (SDS- | | Band present at 97 kDa [%] | 0.3 | 0.3 | 0.2 | 0.5 |
| PAGE non-reducing) | | | | | | |
| Particulate | Subvis. Part. | Particles ≧10 μm | 30 | 6 | 35 | 12 |
| contamination - Sub- | (LO) | [/container] | | | | |
| visible Particles | | Particles ≧25 μm | 1 | 0 | 1 | 1 |
| | | [/container] | | | | |

TABLE 44
Stability of PG110 Lyophilized Formulation 1 Stored at 50° C.

| Test | Procedure | Characteristics | Initial | 1 month |
|---|---|---|---|---|
| Size exclusion HPLC | HPLC | Aggregates [%] | 1.6 | 1.7 |
| (SE-HPLC) | | Fragments [%] | <0.1 | <0.1 |
| | | Monomer [%] | 98.3 | 98.2 |
| Capillary isoelectric | Isoelectric focusing | Sum acidic region [%] | 33.7 | 33.8 |
| focusing (icIEF) | | Main Peak [%] | 56.2 | 53.5 |
| | | Sum basic region [%] | 10.1 | 12.7 |
| SDS gel electrophoresis | SDS-Page (R) | Purity [%] | 99.6 | 99.6 |
| (SDS-PAGE reducing) | | | | |
| SDS gel electrophoresis | SDS-Page (NR) | Purity [%] | 92.3 | 92.6 |
| (SDS-PAGE non- | | Band present at 97 kDa [%] | — | — |
| reducing) | | | | |
| Particulate | Subvis. Part. (LO) | Particles ≧10 μm [/container] | 30 | 1 |
| contamination - Sub- | | Particles ≧25 μm [/container] | 1 | 0 |
| visible Particles | | | | |

TABLE 45
Stability of PG110 Lyophilized Formulation 2 Stored at 2-8° C.

| Test | Procedure | Characteristics | Initial | 1 month | 3 months | 6 months |
|---|---|---|---|---|---|---|
| Size exclusion HPLC | HPLC | Aggregates [%] | 1.9 | 1.9 | 2 | 1.8 |
| (SE-HPLC) | | Fragments [%] | <0.1 | <0.1 | <0.1 | <0.1 |
| | | Monomer [%] | 98.1 | 98 | 98 | 98.2 |
| Capillary isoelectric | Isoelectric | Sum acidic region [%] | 34.2 | 34 | 34 | 31.8 |
| focusing (icIEF) | focusing | Main Peak [%] | 56.4 | 56.5 | 56.5 | 58.5 |
| | | Sum basic region [%] | 9.5 | 9.5 | 9.5 | 9.6 |
| SDS gel | SDS-Page (R) | Purity [%] | 99.6 | 99.4 | 99.5 | 99.5 |
| electrophoresis (SDS- | | | | | | |
| PAGE reducing) | | | | | | |
| SDS gel | SDS-Page (NR) | Purity [%] | 91.9 | 9.,7 | 90.7 | 90.2 |
| electrophoresis (SDS- | | Band present at 97 kDa [%] | 0.3 | 0.2 | 0.3 | 0.1 |
| PAGE non-reducing) | | | | | | |
| Particulate | Subvis. Part. | Particles ≧10 μm | 43 | 6 | 19 | 25 |
| contamination - Sub- | (LO) | [/container] | | | | |
| visible Particles | | Particles ≧25 μm | 1 | 0 | 0 | 0 |
| | | [/container] | | | | |

TABLE 46
Stability of PG110 Lyophilized Formulation 2 Stored at 25° C./60% RH

| Test | Procedure | Characteristics | Initial | 3 months | 6 months |
|---|---|---|---|---|---|
| Size exclusion HPLC | HPLC | Aggregates [%] | 1.9 | 2.2 | 2.4 |
| (SE-HPLC) | | Fragments [%] | <0.1 | <0.1 | <0.1 |
| | | Monomer [%] | 98.1 | 97.8 | 97.6 |
| Capillary isoelectric | Isoelectric focusing | Sum acidic region [%] | 34.2 | 34.1 | 32.6 |
| focusing (icIEF) | | Main Peak [%] | 56.4 | 55.7 | 56.5 |
| | | Sum basic region [%] | 9.5 | 10.2 | 10.9 |
| SDS gel electrophoresis | SDS-Page (R) | Purity [%] | 99.6 | 99.5 | 99.4 |
| (SDS-PAGE reducing) | | | | | |
| SDS gel electrophoresis | SDS-Page (NR) | Purity [%] | 91.9 | 91.9 | 90.1 |
| (SDS-PAGE non- | | Band present at 97 kDa [%] | 0.3 | 0.3 | 0.3 |
| reducing) | | | | | |
| Particulate | Subvis. Part. (LO) | Particles ≧10 μm [/container] | 43 | 28 | 26 |
| contamination - Sub- | | Particles ≧25 μm [/container] | 1 | 0 | 1 |
| visible Particles | | | | | |

TABLE 47
Stability of PG110 Lyophilized Formulation 2 Stored at 40° C./75% RH

| Test | Procedure | Characteristics | Initial | 1 month | 3 months | 6 months |
|---|---|---|---|---|---|---|
| Size exclusion HPLC | HPLC | Aggregates [%] | 1.9 | 3.1 | 5.2 | 8.4 |
| (SE-HPLC) | | Fragments [%] | <0.1 | <0.1 | 0.1 | 0.1 |
| | | Monomer [%] | 98.1 | 96.9 | 94.8 | 91.6 |
| Capillary isoelectric | Isoelectric | Sum acidic region [%] | 34.2 | 34.6 | 33.6 | 39 |
| focusing (icIEF) | focusing | Main Peak [%] | 56.4 | 47.8 | 33.8 | 21.4 |
| | | Sum basic region [%] | 9.5 | 17.6 | 32.6 | 39.6 |
| SDS gel | SDS-Page (R) | Purity [%] | 99.6 | 99.4 | 99.1 | 98.5 |
| electrophoresis (SDS- | | | | | | |
| PAGE reducing) | | | | | | |
| SDS gel | SDS-Page (NR) | Purity [%] | 91.9 | 92.4 | 89.3 | 84.5 |
| electrophoresis (SDS- | | Band present at 97 kDa [%] | 0.3 | 0.2 | 0.2 | 0.2 |
| PAGE non-reducing) | | | | | | |
| Particulate | Subvis. Part. | Particles ≧10 μm | 43 | 4 | 42 | 18 |
| contamination - Sub- | (LO) | [/container] | | | | |
| visible Particles | | Particles ≧25 μm | 1 | 0 | 0 | 0 |
| | | [/container] | | | | |

TABLE 48
Stability of PG110 Lyophilized Formulation 2 Stored at 50° C.

| Test | Procedure | Characteristics | Initial | 1 month |
|---|---|---|---|---|
| Size exclusion HPLC | HPLC | Aggregates [%] | 1.9 | 6.2 |
| (SE-HPLC) | | Fragments [%] | <0.1 | <0.1 |
| | | Monomer [%] | 98.1 | 93.8 |
| Capillary isoelectric | Isoelectric focusing | Sum acidic region [%] | 34.2 | 33.7 |
| focusing (icIEF) | | Main Peak [%] | 56.4 | 33.5 |
| | | Sum basic region [%] | 9.5 | 32.8 |
| SDS gel electrophoresis | SDS-Page (R) | Purity [%] | | |
| (SDS-PAGE reducing) | | | | |
| SDS gel electrophoresis | SDS-Page (NR) | Purity [%] | 91.9 | 93 |
| (SDS-PAGE non- | | Band present at 97 kDa [%] | 0.3 | 0.2 |
| reducing) | | | | |
| Particulate | Subvis. Part. (LO) | Particles ≧10 μm [/container] | 43 | 2 |
| contamination - Sub- | | Particles ≧25 μm [/container] | 1 | 0 |
| visible Particles | | | | |

All data on the samples from Formulations 1 and 2 stored at the intended storage conditions of 2 to 8° C., as well as the samples stored at 25° C. and 40° C. for 6 months meet the acceptance criteria and no significant changes were observed in any of the stability parameters testes at these temperatures. Storage at more extreme stress conditions (50° C.) for one month resulted in a decline in purity which was evident for icIEF only.

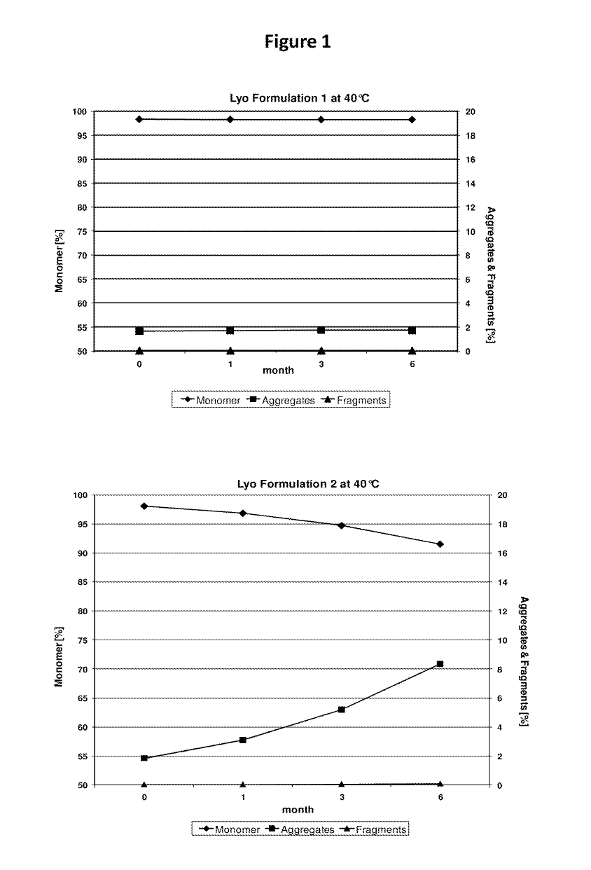

A comparison of Formulations 1 and 2 at 40° C. over 6 months indicated that the PG110 antibody formulated with sucrose alone, demonstrated a higher level of stability than the antibody formulated with a combination of sucrose and mannitol (FIG. 1). In addition, it was surprisingly observed that that the formation of subvisible and visible particles in these formulations, which contain a molar ratio of sugar and/or polylol:protein greater than 1400 (e.g., Formulation 1—protein:sugar=1:1515; Formulation 2—protein:sugar+polylol ratio=1436), does not change over time, even at accelerated stability studies at 40° C.

### INCORPORATION BY REFERENCE

The present invention incorporates by reference in their entirety techniques well known in the field of protein formulation. These techniques include, but are not limited to, techniques described in the following publications: Ausubel et al. (eds.), Current Protocols in Molecular Biology, John Wiley & Sons, NY (1993); Ausubel, F. M. et al. eds., Short Protocols In Molecular Biology (4th Ed. 1999) John Wiley & Sons, NY. (ISBN 0-471-32938-X). Controlled Drug Bioavailability Drug Product Design and Performance, Smolen and Ball (eds.), Wiley, New York (1984); Giege, R. and Ducruix, A. Barrett, Crystallization of Nucleic Acids and Proteins, a Practical Approach, 2nd ea., pp. 20 1-16, Oxford University Press, New York, N.Y., (1999); Goodson, in Medical Applications of Controlled Release, vol. 2, pp. 115-138 (1984); Hammerling, et al., in: Monoclonal Antibodies and T-Cell Hybridomas 563-681 (Elsevier, N.Y., 1981; Harlow et al., Antibodies: A Laboratory Manual, (Cold Spring Harbor Laboratory Press, 2nd ed. 1988); Kabat et al., Sequences of Proteins of Immunological Interest (National Institutes of Health, Bethesda, Md. (1987) and (1991); Kabat, E. A., et al. (1991) Sequences of Proteins of Immunological Interest, Fifth Edition, U.S. Department of Health and Human Services, NIH Publication No. 91-3242; Kontermann and Dubel eds., Antibody Engineering (2001) Springer-Verlag. New York. 790 pp. (ISBN 3-540-41354-5); Kriegler, Gene Transfer and Expression, A Laboratory Manual, Stockton Press, NY (1990); Lu and Weiner eds., Cloning and Expression Vectors for Gene Function Analysis (2001) BioTechniques Press. Westborough, Mass. 298 pp. (ISBN 1-881299-21-X), Medical Applications of Controlled Release, Langer and Wise (eds.), CRC Pres., Boca Raton, Fla. (1974); Old, R. W. & S. B. Primrose, Principles of Gene Manipulation: An Introduction To Genetic Engineering (3d Ed. 1985) Blackwell Scientific Publications, Boston. Studies in Microbiology; V.2:409 pp. (ISBN 0-632-01318-4); Sambrook, J. et al. eds., Molecular Cloning: A Laboratory Manual (2d Ed. 1989) Cold Spring Harbor Laboratory Press, NY. Vols. 1-3 (ISBN 0-87969-309-6); Sustained and Controlled Release Drug Delivery Systems, J. R. Robinson, ed., Marcel Dekker, Inc., New York, 1978; Winnacker, E. L. From Genes To Clones: Introduction To Gene Technology (1987) VCH Publishers, N.Y. (translated by Horst Ibelgaufts). 634 pp. (ISBN 0-89573-614-4).

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes

We claim:

1. A pharmaceutical composition comprising:
(a) an anti-nerve growth factor (NGF) antibody, or antigen binding fragment thereof;
(b) a histidine buffer at a concentration of about 5 to about 60 mM; and
(c) polysorbate 80 at a concentration of about 0.01% to about 0.1%;
wherein the pH of the composition is about 5.0 to about 6.0.

2. The pharmaceutical composition of claim 1, wherein the composition further comprises about 1 to about 100 mg/mL of a polyol.

3. The pharmaceutical composition of claim 1, wherein the composition further comprises about 10 to about 100 mg/mL of a sugar.

4. The pharmaceutical composition of claim 1, wherein the concentration of the antibody, or antigen-binding portion thereof, is about 1 to about 240 mg/mL.

5. The pharmaceutical composition of claim 2 or 3, wherein the molar ratio of (a) anti-NGF antibody, or antigen binding fragment thereof, to (b) polyol, sugar, or combination thereof, is greater than 1:1400.

6. The pharmaceutical composition of claim 1, wherein said composition comprises:
(a) about 20 mg/mL of the antibody, or antigen-binding portion thereof;
(b) about 15 mM histidine; and
(c) about 0.01% polysorbate 80;
wherein the pH of the formulation is about 5.5.

7. The pharmaceutical composition of claim 1, wherein said composition comprises:
(a) about 60 mg/mL of the antibody, or antigen-binding portion thereof;
(b) about 30 mM histidine; and
(c) about 0.02% polysorbate 80;
wherein the pH of the formulation is about 5.5.

8. The pharmaceutical composition of claim 1, wherein the composition is lyophilized.

9. The lyophilized pharmaceutical composition of claim 8, comprising:
(a) about 1 to about 120 mg of an anti-NGF antibody, or antigen binding fragment thereof;
(b) about 1 to about 10 mg of histidine; and
(c) about 0.1 to about 0.4 mg of polysorbate 80.

10. The lyophilized pharmaceutical composition of claim 9, further comprising about 1 to about 100 mg of a polyol.

11. The lyophilized pharmaceutical composition of claim 9, further comprising about 1 to about 100 mg of a sugar.

12. The pharmaceutical composition of claim 1, wherein the anti-NGF antibody, or antigen-binding portion thereof, binds to human NGF.

13. The pharmaceutical composition of claim 12, wherein the anti-NGF antibody, or antigen-binding portion thereof, comprises a human IgG4 constant region.

14. The pharmaceutical composition of claim 13, wherein the anti-NGF antibody, or antigen-binding portion comprises a hinge region mutation.

15. The pharmaceutical composition of claim 14, wherein the hinge region mutation comprises a mutation of a serine at amino acid position 108 of SEQ ID NO: 9.

16. The pharmaceutical composition of claim 14, wherein the human IgG4 constant region comprises the amino acid sequence of SEQ ID NO: 10.

17. The pharmaceutical composition of claim 12, wherein the antibody, or antigen-binding portion thereof, has one or more of the following functional properties:
a) binds to human NGF but does not bind to human brain-derived neurotrophic factor (BDNF), human neurotrophin 3 (NT-3) or human neurotrophin 4 (NT-4);
b) binds to human or rat NGF with a K_{D} of 100 pM or less;
c) inhibits binding of NGF to TrkA or p75^{NTR};
d) inhibits NGF-dependent proliferation of TF-1 cells;
e) inhibits NGF-dependent chick dorsal root ganglion survival; and
f) inhibits NGF-dependent PC12 cell neurite outgrowth.

18. The pharmaceutical composition of claim 12, wherein the antibody, or antigen-binding portion thereof, does not exhibit a rebound effect when administered to a subject.

19. The pharmaceutical composition of claim 1, wherein the antibody, or antigen-binding portion thereof, comprises a heavy chain variable region comprising CDRs 1, 2 and 3, having the amino acid sequences of SEQ ID NOs: 3, 4 and 5, respectively.

20. The pharmaceutical composition of claim 1, wherein the antibody, or antigen-binding portion thereof, comprises a light chain variable region comprising CDRs 1, 2 and 3, having the amino acid sequences of SEQ ID NOs: 6, 7 and 8, respectively.

21. The pharmaceutical composition of claim 1, wherein the antibody, or antigen-binding portion thereof, comprises a heavy chain variable region comprising the amino acid sequence of SEQ ID NO: 1.

22. The pharmaceutical composition of claim 1, wherein the antibody, or antigen-binding portion thereof, comprises a light chain variable region comprising the amino acid sequence of SEQ ID NO: 2.

23. The pharmaceutical composition of claim 1, wherein the antibody, or antigen-binding portion thereof, competes for binding to NGF with an antibody comprising a heavy chain variable region comprising the amino acid sequence of SEQ ID NO: 1 and a light chain variable region comprising the amino acid sequence of SEQ ID NO: 2.

24. The pharmaceutical composition of claim 1, wherein the antibody, or antigen-binding portion thereof, comprises a heavy chain comprising the amino acid sequence of SEQ ID NO: 13.

25. The pharmaceutical composition of claim 1, wherein the antibody, or antigen binding portion thereof, comprises a light chain comprising the amino acid sequence of SEQ ID NO: 16.

26. A pharmaceutical composition comprising:
(a) an anti-nerve growth factor (NGF) antibody comprising a human IgG4 constant region, wherein the antibody comprises a heavy chain having the amino acid sequence of SEQ ID NO:13 and a light chain having the amino acid sequence of SEQ ID NO:16, wherein the concentration of the antibody, or antigen binding fragment thereof, is about 10 to about 50 mg/mL;
(b) a histidine buffer at a concentration of about 10 to about 30 mM histidine; and
(c) polysorbate 80 at a concentration of about 0.01% to 0.02%;
wherein the pH of the composition is about 5.0 to about 6.0.

27. The pharmaceutical composition of claim 26, further comprising about 10 to about 50 mg/mL mannitol.

28. The pharmaceutical composition of claim 26, further comprising about 5 to about 70 mg/mL sucrose.

29. The pharmaceutical composition of claim 26, consisting essentially of:
(a) about 10 to 30 mg/mL of the antibody or antigen-binding fragment thereof;
(b) about 15 mM histidine buffer; and
(c) about 0.01% polysorbate 80;
wherein the pH of the composition is about 5.5.

30. The pharmaceutical composition of claim 27, consisting essentially of:
(a) about 10 to 30 mg/mL of the antibody or antigen-binding fragment thereof;
(b) about 15 mM histidine buffer;
(c) about 0.01% polysorbate 80; and
(d) about 10 to 30 mg/mL mannitol;
wherein the pH of the composition is about 5.5.

31. The pharmaceutical composition of claim 28, consisting essentially of:
(a) about 10 to 30 mg/mL of the antibody or antigen-binding fragment thereof;
(b) about 15 mM histidine buffer;
(c) about 0.01% polysorbate 80; and
(d) about 40 to 70 mg/mL sucrose;
wherein the pH of the composition is about 5.5.

32. The pharmaceutical composition of claim 28, consisting essentially of:
(a) about 10 to 30 mg/mL of the antibody or antigen-binding fragment thereof;
(b) about 15 mM histidine buffer;
(c) about 0.01% polysorbate 80;
(d) about 10 to 30 mg/mL mannitol; and
(e) about 5 to 10 mg/mL sucrose;
wherein the pH of the composition is about 5.5.

33. The pharmaceutical composition of any one of claim 27 or 28, wherein the ratio of (a) antibody, or antigen binding fragment thereof, to (b) polyol, sugar, or combination thereof, is greater than 1:1400.

34. The pharmaceutical composition of claim 26, wherein the pharmaceutical composition is lyophilized.

35. The pharmaceutical composition of claim 1, wherein the antibody, or antigen-binding portion thereof, is selected from the group consisting of a monoclonal antibody, a human antibody, a humanized antibody, a chimerical antibody, a CDR-grafted antibody, a Fab, a Fab′, a F(ab′)2, a Fv, a disulfide linked Fv, a scFv, a single domain antibody, a diabody, a multispecific antibody, a dual specific antibody, and a bispecific antibody.

36. The pharmaceutical composition of claim 1, wherein the formulation is stable in a liquid form for at least about 3 months at 2-25° C.

37. The pharmaceutical composition of claim 1, wherein the formulation is stable for at least 6 months in frozen or lyophilized form.

38. The pharmaceutical composition of claim 37, wherein the formulation is stored frozen at −80° C.

39. The pharmaceutical composition of claim 37, wherein the formulation is stored in lyophilized form at 2-25° C.

40. The pharmaceutical composition of claim 36 or 37, wherein there is less than about 10% aggregation of the antibody.

41. The pharmaceutical composition of claim 1, wherein the formulation is suitable for intravenous, subcutaneous and/or intramuscular administration.

42. A device comprising the pharmaceutical composition of any claim 1.

43. The device of claim 42, wherein the device is selected from the group consisting of a syringe, a pen, an implant, a needle-free injection device, an inhalation device, and a patch.

44. A kit comprising the pharmaceutical composition of claim 1 or device of claim

45. A method of attenuating or inhibiting an NGF mediated disease or condition in a subject, the method comprising administering to the subject the pharmaceutical composition of claim 1.

46. The method of claim 45, wherein the NGF mediated disease or condition is pain.

47. The method of claim 45, wherein the pharmaceutical composition is suitable for administration intravenously, subcutaneously or intra-articularly.

48. The method of claim 45, wherein the pharmaceutical composition is suitable for administration with a second pharmaceutical agent.


SEQUENCE LISTING (16 sequences)

SEQ ID NO: 1 - protein, 122 aa, Artificial Sequence
  Description of Artificial Sequence Synthetic polypeptide
EVQLVESGGGLVQPGGSLRLSCAASGFSLTNNNVNWVRQAPGKGLEWVGGVWAGGATDYNSALKSRFTISRDNSKNTAYLQMNSLRAEDTAVYYCARDGGYSSSTLYAMDAWGQGTLVTVSS

SEQ ID NO: 2 - protein, 107 aa, Artificial Sequence
  Description of Artificial Sequence Synthetic polypeptide
DIQMTQSPSSLSASVGDRVTITCRASEDIYNALAWYQQKPGKAPKLLIYNTDTLHTGVPSRFSGSGSGTDYTLTISSLQPEDFATYFCQHYFHYPRTFGQGTKVEIK

SEQ ID NO: 3 - protein, 10 aa, Artificial Sequence
  Description of Artificial Sequence Synthetic peptide
GFSLTNNNVN

SEQ ID NO: 4 - protein, 16 aa, Artificial Sequence
  Description of Artificial Sequence Synthetic peptide
GVWAGGATDYNSALKS

SEQ ID NO: 5 - protein, 14 aa, Artificial Sequence
  Description of Artificial Sequence Synthetic peptide
DGGYSSSTLYAMDA

SEQ ID NO: 6 - protein, 11 aa, Artificial Sequence
  Description of Artificial Sequence Synthetic peptide
RASEDIYNALA

SEQ ID NO: 7 - protein, 7 aa, Artificial Sequence
  Description of Artificial Sequence Synthetic peptide
NTDTLHT

SEQ ID NO: 8 - protein, 9 aa, Artificial Sequence
  Description of Artificial Sequence Synthetic peptide
QHYFHYPRT

SEQ ID NO: 9 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 327 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - DNA, 1407 bp, Artificial Sequence
  Description of Artificial Sequence Synthetic polynucleotide
atggaatggagctgggtgttcctgttcttcctgagcgtgaccaccggcgtgcacagcgaggtgcagctggtcgagagcggcggagggctggtgcagccaggcggcagcctgaggctgtcctgcgccgccagcggcttcagcctgaccaacaacaacgtgaactgggtgcggcaggccccaggcaagggcctggaatgggtgggcggcgtgtgggccgggggagccaccgactacaacagcgccctgaagagcaggttcaccatcagcagggacaacagcaagaacaccgcctacctgcagatgaacagcctgagggccgaggacaccgccgtgtactactgcgccagggacggcggctacagcagcagcaccctgtacgccatggacgcctggggccagggcaccctggtgaccgtgagcagcgccagcaccaagggccccagcgtgttccccctggccccctgcagcagaagcaccagcgagagcacagccgccctgggctgcctggtgaaggactacttccccgagcccgtgaccgtgtcctggaacagcggagccctgaccagcggggtgcacaccttccccgccgtgctgcagagcagcggcctgtacagcctgagcagcgtggtgacagtgcccagcagcagcctgggcaccaagacctacacctgcaacgtggaccacaagcccagcaacaccaaggtggacaagagggtggagagcaagtacggcccaccctgccccccatgcccagcccccgagttcctgggcggaccctccgtgtttctgttcccccccaagcccaaggacaccctgatgatcagcaggacccccgaggtgacctgcgtggtggtggacgtgagccaggaagatccagaggtccagttcaactggtacgtggacggcgtggaggtgcacaacgccaagaccaagcccagagaggaacagtttaacagcacctacagggtggtgtccgtgctgaccgtgctgcaccaggactggctgaacggcaaggagtacaagtgcaaggtctccaacaagggcctgcccagctccatcgagaaaaccatcagcaaggccaagggccagccacgggagccccaggtgtacaccctgccaccctcccaggaagagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaggctgaccgtggacaagtccaggtggcaggaaggcaacgtctttagctgcagcgtgatgcacgaggccctgcacaaccactacacccagaagagcctgtccctgagcctgggcaagtga

SEQ ID NO: 12 - protein, 468 aa, Artificial Sequence
  Description of Artificial Sequence Synthetic polypeptide
MEWSWVFLFFLSVTTGVHSEVQLVESGGGLVQPGGSLRLSCAASGFSLTNNNVNWVRQAPGKGLEWVGGVWAGGATDYNSALKSRFTISRDNSKNTAYLQMNSLRAEDTAVYYCARDGGYSSSTLYAMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 449 aa, Artificial Sequence
  Description of Artificial Sequence Synthetic polypeptide
EVQLVESGGGLVQPGGSLRLSCAASGFSLTNNNVNWVRQAPGKGLEWVGGVWAGGATDYNSALKSRFTISRDNSKNTAYLQMNSLRAEDTAVYYCARDGGYSSSTLYAMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - DNA, 705 bp, Artificial Sequence
  Description of Artificial Sequence Synthetic polynucleotide
atgagcgtgcccacccaggtgctgggcctgctgctgctgtggctgaccgacgccagatgcgacatccagatgacccagagccccagcagcctgagcgccagcgtgggcgacagggtgaccatcacctgcagggccagcgaggacatctacaacgccctggcctggtatcagcagaagcccggcaaggcccccaagctgctgatctacaacaccgacaccctgcacaccggcgtgcccagcaggttcagcggcagcggctccggcaccgactacaccctgaccatcagcagcctgcagcccgaggacttcgccacctacttttgccagcactacttccactaccccaggaccttcggccagggcaccaaggtggagatcaagaggaccgtggctgcccccagcgtgttcatcttcccccccagcgacgagcagctgaagagcggcaccgcctccgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgctga

SEQ ID NO: 15 - protein, 234 aa, Artificial Sequence
  Description of Artificial Sequence Synthetic polypeptide
MSVPTQVLGLLLLWLTDARCDIQMTQSPSSLSASVGDRVTITCRASEDIYNALAWYQQKPGKAPKLLIYNTDTLHTGVPSRFSGSGSGTDYTLTISSLQPEDFATYFCQHYFHYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 214 aa, Artificial Sequence
  Description of Artificial Sequence Synthetic polypeptide
DIQMTQSPSSLSASVGDRVTITCRASEDIYNALAWYQQKPGKAPKLLIYNTDTLHTGVPSRFSGSGSGTDYTLTISSLQPEDFATYFCQHYFHYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC